(19) United States
(12) Patent Application Publication
(10) Pub. No.: US 2005/0246799 A1
(43) Pub. Date: Nov. 3, 2005

(54) Receptors for hypersensitive response elicitors and uses thereof

(76) Inventors: Xiaoling Song, Woodinville, WA (US); Pauline Anne Bariola, Seattle, WA (US); Nora Abiella Linderoth, Kenmore, WA (US); Hao Fan, Bothell, WA (US); Zhong-Min Wei, Kirkland, WA (US)

Correspondence Address: Michael L. Goldman;Nixon Peabody LLP, Clinton Square, P.O. Box 31051, Rochester, NY, 14603-1051, US

(21) Appl. No.: 10/972,587

(22) Filed: Oct. 25, 2004

Related U.S. Application Data
(62) Division of application No. 10/174,209, filed on Jun. 17, 2002, now abandoned.
Application No. 10/174,209 is a continuation-in-part of application No. 09/810,997, filed on Mar. 16, 2001, now abandoned.
(60) Provisional application No. 60/335,776, filed on Oct. 31, 2001.

(51) Int. Cl. A01H 5/00; C12Q 1/00; C07H 21/04; C12N 15/82; A01H 1/00

(52) U.S. Cl. 800/301; 530/370; 536/23.6; 435/320.1; 435/419; 800/279; 435/4

(57) ABSTRACT

The present invention is directed to an isolated protein which serves as a receptor in plants for a plant pathogen hypersensitive response elicitor. Also disclosed are nucleic acid molecules encoding such receptors as well as expression vectors, host cells, transgenic plants, and transgenic plant seeds containing such nucleic acid molecules. Both the protein and nucleic acid can be used to identify agents targeting plant cells to enhance a plant's receptivity to treatment with a hypersensitive response elicitor and to directly impart plant growth enhancement as well as resistance against disease, insects, and stress.

The present application is a divisional of U.S. patent application Ser. No. 10/174,209, filed Jun. 17, 2002, which is hereby incorporated by reference in its entirety and is a continuation-in-part of U.S. patent application Ser. No. 09/810,997, filed Mar. 16, 2001, and claims benefit of U.S. Provisional Patent Application Serial No. 60/335,776, filed Oct. 31, 2001.

### FIELD OF THE INVENTION

The present invention relates to receptors for hypersensitive response elicitors and uses thereof.

### BACKGROUND OF THE INVENTION

Plants have evolved a complex array of biochemical pathways that enable them to recognize and respond to environmental signals, including pathogen infection. There are two major types of interactions between a pathogen and plant—compatible and incompatible. When a pathogen and a plant are compatible, disease generally occurs. If a pathogen and a plant are incompatible, the plant is usually resistant to that particular pathogen. In an incompatible interaction, a plant will restrict pathogen proliferation by causing localized necrosis, or death of tissues, to a small zone surrounding the site of infection. This reaction by the plant is defined as the hypersensitive response (“HR”) (Kiraly, Z. “Defenses Triggered by the Invader: Hypersensitivity,” Plant Disease: An Advanced Treatise 5:201-224 J. G. Horsfall and E. B. Cowling, eds. Academic Press, New York (1980); (Klement “Hypersensitivity,” Phytopathogenic Prokaryotes 2:149-177, M. S. Mount and G. H. Lacy, eds. Academic Press, New York (1982)). The localized cell death not only contains the infecting pathogen from spreading further but also leads to a systemic resistance preventing subsequent infections by other pathogens. Therefore, HR is a common form of plant resistance to diseases caused by bacteria, fungi, nematodes, and viruses.

A set of genes designated as hrp (Hypersensitive Response and Pathogenicity) is responsible for the elicitation of the HR by pathogenic bacteria, including Erwinia spp, Pseudomonas spp, Xanthomonas spp, and Ralstonia solanacearum (Willis et al. “hrp Genes of Phytopathogenic Bacteria,” Mol. Plant-Microbe Interact. 4:132-138 (1991), Bonas, U. “hrp Genes of Phytopathogenic Bacteria,” pages 79-98 in: Current Topics in Microbiology and Immunology, Vol. 192, Bacterial Pathogenesis of Plants and Animals: Molecular and Cellular Mechanisms. J. L. Dangl, ed. Springer-Verlag, Berlin (1994); Alfano et al., “Bacterial Pathogens in Plants: Life Up Against the Wall,” Plant Cell 8:1683-98 (1996). Typically, there are multiple hrp genes clustered in a 30-40 kb segments of DNA. Mutation in any one of the hrp genes will result in the loss of bacterial pathogenicity in host plants and the HR in non-host plants. On the basis of genetic and biochemical characterization, the function of the hrp genes can be classified into three groups: 1) structural genes encoding extracellularly located HR elicitors, for example harpin of Erwinia amylovora (Wei et al. “Harpin, Elicitor of the Hypersensitive Response Produced by the Plant Pathogen Erwinia amylovora,” Science 257:85 (1992)); 2) secretion genes encoding a secretory apparatus for exporting HR elicitors and other proteins from the bacterial cytoplasm to the cell surface or extracellular space (Van Gijsegem et al., “Evolutionary Conservation of Pathogenicity Determinants Among Plant and Animal Pathogenic Bacteria,” Trends Microbiol. 1 :175-180 (1993); He et al, “Pseudomonas syringae pv. Syringae harpinₚₛₛ: A Protein that is Secreted Via the Hrp Pathway and Elicits the Hypersensitive Response in Plants,” Cell 73:1255 (1993); Wei et al., “HrpI of Erwinia amylovora Functions in Secretion of Harpin and is a Member of a New Protein Family,” J. Bacteriol. 175:7985-67 (1993), Arlat et al. “PopA1, a Protein which Induces a Hypersensitive-Like Response on Specific Petunia Genotypes, is Secreted via the Hrp Pathway of Pseudomonas solanacearum,” EMBO J. 13:543-53 (1994), Galan et al., “Cross-talk between Bacterial Pathogens and their Host Cells,” Ann. Rev. Cell Dev. Biol. 12:221-55 (1996); Bogdanove et al., “Erwinia amylovora Secretes Harpin via a Type III Pathway and Contains a Homolog of yopN of Yersinia,” J. Bacteriol. 178:1720-30 (1996); Bogdanove et al., “Homology and Functional Similarity of a hrp-linked Pathogenicity Operon, dspEF, of Erwinia amylovora and the avrE locus of Pseudomonas syringae pathovar tomato,” Proc. Natl. Acad. Sci. USA 95:1325-30 (1998)); and 3) regulatory genes that control the expression of hrp genes (Wei, Z. M., “Harpin, Elicitor of the Hypersensitive Response Produced by the Plant Pathogen Erwinia amylovora,” Science 257:85 (1992); Wei et al., “hrpL Activates Erwinia amylovora hrp Genes in Response to Environmental Stimuli,” J. Bacteriol. 174:1875-82 (1995); Xiao et al., “A Single Promoter Sequence Recognized by a Newly Identified Alternate Sigma Factor Directs Expression of Pathogenicity and Host Range Determinants in Pseudomonas syringae,” J. Bacteriol. 176:3089-91 (1994); Kim et al., “The hrpA and hrpC Operons of Erwinia amylovora Encode Components of a Type III Pathway that Secretes Harpin,” J. Bacteriol. 179:1690-97 (1997); Kim et al., “HrpW of Erwinia amylovora, a New Harpin that Contains a Domain Homologous to Pectate Lyases of a Distinct Class,” J. Bacteriol. 180:5203-10 (1998); Wengelnik et al., “HrpG, A Key hrp Regulatory Protein of Xanthomonas campestris pv. Vesicatoria is Homologous to Two Component Response Regulators,” Mol. Plant-Microbe Interact. 9:704-12 (1996)). Because of their role in interactions between plants and microbes, hrp genes have been a focus for bacterial pathogenicity and plant defense studies.

In addition to the local defense response, HR also activates the defense system in uninfected parts of the same plant. This results in a general systemic resistance to a secondary infection termed Systemic Acquired Resistance (“SAR”) (Ross, R. F. “Systemic Acquired Resistance Induced by Localized Virus Infections in Plants,” Virology 14:340-58 (1961); Malamy et al., “Salicylic Acid and Plant Disease Resistance,” Plant J. 2:643-654 (1990)). SAR confers long-lasting systemic disease resistance against a broad spectrum of pathogens and is associated with the expression of a certain set of genes (Ward et al. “Coordinate Gene Activity in Response to Agents that Induce Systemic Acquired Resistance,” Plant Cell 3:1085-94 (1991)). SAR is an important component of the disease resistance of plants and has long been of interest, because the potential of inducing the plant to protect itself could significantly reduce or eliminate the need for chemical pesticides. SAR can be induced by biotic (microbes) or abiotic (chemical) agents (Gorlach et al. “Benzothiadiazole, a Novel Class of Inducers of Systemic Acquired Resistance, Activates Gene Expression and Disease Resistance in Wheat,” Plant Cell 8:629-43 (1996)). Historically, weak virulent pathogens were used as a biotic inducing agent for SAR. Non-virulent plant growth promotion bacteria (“PGPR”) were also reported to be able to induce resistance of some plants against various diseases. Biotic agent-induced SAR has been the subject of much research, especially in the late 70s and early 80s. Only very limited success was achieved, however, due to: 1) inconsistency of the performance of living organisms in different environmental conditions; 2) considerable concerns regarding the unpredictable consequences of the intentional introduction of weakly virulent pathogens into the environment; and 3) the technical complication of applying a living microorganism into a variety of environmental conditions. To overcome the limitations of using living organisms to induce SAR, scientists have long been looking for an HR elicitor derived from a pathogen for SAR induction. With the advancement of molecular biology, the first proteinaceous HR elicitor with broad host spectrum was isolated in 1992 from Erwinia amylovora, a pathogenic bacterium causing fire blight in apple and pear. The HR elicitor was named “harpin”. It consists of 403 amino acids with a molecular weight about 40 kDa. The harpin protein is heat-stable and glycine-rich with no cysteine. The gene encoding the harpin protein is contained in a 1.3 kb DNA fragment located in the middle of the hrp gene cluster. Harpin is secreted into the extracellular space and is very sensitive to proteinase digestion. Since the first harpin was isolated from Erwinia amylovora, several harpin or harpin-like proteins have been isolated from other major groups of plant pathogenic bacteria. In addition to the harpin of Erwinia amylovora, the following harpin or harpin-like proteins have been isolated and characterized: HrpN of Erwinia chrysanthemi, Erwinia carotovora (Wei et al. “Harpin, Elicitor of the Hypersensitive Response Produced by the Plant Pathogen Erwinia amylovora,” Science 257:85 (1992)), and Erwinia stewartii; HrpZ of Pseudomonas syringae (He et al, “Pseudomonas syringae pv. Syringae harpinₚₛₛ: A Protein that is Secreted Via the Hrp Pathway and Elicits the Hypersensitive Response in Plants,” Cell 73:1255 (1993)), PopA of Ralstonia solanacearum, (Arlat et al. “PopA1, a Protein which Induces a Hypersensitive-Like Response on Specific Petunia Genotypes, is Secreted via the Hrp Pathway of Pseudomonas solanacearum,” EMBO J. 13:543-53 (1994)); HrpW of Erwinia amylovora (Kim et al., “HrpW of Erwinia amylovora, a New Harpin that Contains a Domain Homologous to Pectate Lyases of a Distinct Class,” J. Bacteriol. 180:5203-10 (1998)), and Pseudomonas syringae. All of the currently described harpin or harpin-like proteins share common characteristics. They are heat-stable and glycine-rich proteins with no cysteine amino acid residue, are very sensitive to digestion by proteinases, and elicit the HR and induce resistance in many plants against many diseases. Based on their shared biochemical and biophysical characteristics as well as biological functions, these HR elicitors from different pathogenic bacteria belong to a new protein family—i.e. the harpin protein family. The described characteristics, especially their ability to induce HR in a broad range of plants, distinguish the harpin protein family from other host specific proteinaceous HR elicitors, for example elicitins from Phytophthora spp (Bonnet et al., “Acquired Resistance Triggered by Elicitors in Tobacco and Other Plants,” Eur. J. Plant Path. 102:181-92 (1996); Keller, et al. “Physiological and Molecular Characteristics of Elicitin-Induced Systemic Acquired Resistance in Tobacco,” Plant Physiol 110:365-76 (1996)) or avirulence proteins (such as Avr9) from Cladosporium fulvum, which are only able to elicit the HR in a specific variety or species of a plant.

In nature, when certain bacterial infections occur, harpin protein is expressed and then secreted by the bacteria, signaling the plant to mount a defense against the infection. Harpin serves as a signal to activate plant defense and other physiological systems, which include SAR, growth enhancement, and resistance to certain insect damage.

The current understanding of critical plant molecules that may have a significant role in interacting with elicitors and then triggering a sequential signal transduction cascade is described as follows.

Interaction of Plant Resistance Genes (R) and Pathogen Avirulence Genes (avr)

The concept of gene-for-gene interaction is that “for each gene determining resistance (R gene) in the host, there is a corresponding gene determining avirulence in the pathogen (avr gene)”. In this model, pathogen avirulence genes generate a specific ligand molecule, called an elicitor. Only plants carrying the matching resistance gene respond to this elicitor and invoke the HR. In the past few years, several disease-resistance, R genes, have been cloned and sequenced. It was expected that R genes might encode components involved in signal recognition or signal transduction pathways that ultimately lead to defense responses. The cloned R genes could be grouped into four classes: (1) cytoplasmic protein kinase; (2) protein kinases with an extracellular domain; (3) cytoplasmic proteins with a region of leucine-rich repeats and a nucleotide-binding site; and (4) proteins with a region of leucine-rich repeats that appear to encode extracellular proteins. (Review in Bent, A. F. “Plant Disease Resistance Genes: Function Meets Structure,” Plant Cell 8:1757-71 (1996); Baker B., et al., “Signaling in Plant-Microbe Interactions,” Science 276:726-33 (1997)). The first R gene cloned, Pto, encodes a serine/threonine protein kinase. The protein product of Pto directly interacts with the cognate avirulence gene protein, AvrPro, which has been demonstrated in a yeast two-hybrid system. It was shown that only co-existence of both AvrPro and Pto proteins could elicit HR in plants (Tang et al., “Initiation of Plant Disease Resistance by Physical Interaction of AvrPto and Pto kinase,” Science 274:2060-63 (1996); Scofield et al., “Molecular Basis of Gene-for-Gene Specificity in Bacterial Speck Disease of Tomato,” Science 274:2063-65 (1996); Zhou et al., “The Pto Kinase Conferring Resistance to Tomato Bacterial Speck Disease Interacts with Proteins that Bind a cis-element of Pathogenesis-related Genes,” EMBO J. 16:3207-18 (1997)). The results from cloned R genes support the view that plant-pathogen interactions involve protein-protein interactions. Syringolide, a water-soluble, low-molecular-weight elicitor, triggers a defense response in soybean cultivars carrying the Rpg4 disease-resistance gene. A 34-KDa protein has been isolated from soybean and is considered to be the physiological active syringolide receptor (Ji et al., “Characterization of a 34-kDa Soybean Binding Protein for the Syringolide Elicitors,” Proc. Natl. Acad. Sci. USA 95:3306-11 (1998)).

Putative Binding Factor of Elicitin

Elicitins are a family of small proteins secreted by Phytophthora species that have a high degree of homology. Pure elicitins alone can cause a hypersensitive response, a local cell death, and trigger systemic acquired resistance in tobacco and other plants (Bonnet et al., “Acquired Resistance Triggered by Elicitors in Tobacco and Other Plants,” Eur. J. Plant Path. 102:181-92 (1996); Keller, et al. “Physiological and Molecular Characteristics of Elicitin-Induced Systemic Acquired Resistance in Tobacco,” Plant Physiol 110:365-76 (1996)). However, the spectrum of HR elicitation and induced systemic resistance in plants is much narrower than that achieved by harpin family elicitors. Like harpin, elicitins induce a series of metabolic events in tobacco cells, including the accumulation of phytoalexins, ethylene production, transmembrane electrolyte leakage, H₂O₂ accumulation, and expression of plant defense related genes (Yu L, et al., “Elicitins from Phytophthora and Basic Resistance in Tobacco,” Proc. Natl. Acad. Sci. (1995); Keller et al., “Pathogen-Induced Elicitin Production in Transgenic Tobacco Generates a Hypersensitive Response and Nonspecific Disease Resistance,” The Plant Cell 11:223-35 (1999)). A putative receptor-like binding factor has been identified in tobacco plasma membrane, which has a specific high-affinity to the crytogein, one member of the elicitin family (Wendehenne, et al., “Evidence for Specific, High-Affinity Binding Sites for a Proteinaceous Elicitor in Tobacco Plasma Membrane,” FEBS Letters 374:203-207 (1995)). Recently, it was found that 2 basic elicitins (i.e. cryptogein and cinnamomin) and two acidic elicitins (i.e. capsicein and parasiticein) were able to interact with the same binding sites on tobacco plasma membranes (Bourque et al., “Comparison of Binding Properties and Early Biological Effects of Elicitins in Tobacco Cells,” Plant Physiol. 118:1317-26 (1998)). However, the gene of the receptor-like factor has not been isolated.

Putative Binding Factor of Glycoprotein Elicitors

A 42 kDa glycoprotein elicitor has been isolated from Phytophthora megasperma (Parker et al., “An Extracellular Glycoprotein from Phytophthora megasperma f. sp. glycinea Elicits Phytoalexin Synthesis in Cultured Parsley Cells and Protoplasts,” Mol. Plant Microbe Interact. 4:19-27 (1991)). An oligopeptide of 13 amino acids within the glycoprotein (“Pep-13”) was able to induce a response in plants like that achieved by the full glycoprotein. A high affinity-binding pattern has been observed in parsley microsomal membranes with an isotope labeled oligopeptide. There are estimated to be about 1600 to 2900 binding sites per cell with evidence indicating that a low abundance protein receptor of the Pep-13 is localized in the plasma membrane (Numberger et al., “High Affinity Binding of a Fungal Oligopeptide Elicitor to Parsley Plasma Membranes Triggers Multiple Defense Responses,” Cell 78:449-60 (1994)).

Harpin Protein Binding Factors

Harpin proteins, which elicit HR in a variety of different nonhost plants, have been isolated from plant pathogens (Wei et al. “Harpin, Elicitor of the Hypersensitive Response Produced by the Plant Pathogen Erwinia amylovora,” Science 257:85 (1992)). A family of harpin proteins has been identified from plant bacterial pathogens. All of them have similar biological activities. It is well documented that harpin protein can induce plants to produce active oxygen, change ion flux, lead to local cell death, and induce systemic acquired resistance (“SAR”) (Wei et al. “Harpin, Elicitor of the Hypersensitive Response Produced by the Plant Pathogen Erwinia amylovora,” Science 257:85 (1992); He et al., “Pseudomonas syringae pv. syringae Harpinₚₛₛ: A Protein that is Secreted via the Hrp Pathway and Elicits the Hypersensitive Response in Plants,” Cell 73:1255-66 (1993); Baker, C. J., et al., “Harpin, an Elicitor of the Hypersensitive Response in Tobacco Caused by Erwinia amylovora, Elicits Active Oxygen Production in Suspension Cells,” Plant Physiol. 102:1341-44 (1993)). No harpin protein binding factor has been isolated so far. It was reported that an amphipathic protein, named HRAP, isolated from sweet pepper could dissociate harpingₚₚₛ in multimeric form (hrpZ from Pseduomonas syringae). The biological activity of the HRAP is believed to be its ability to intensify harpinₚₛₛ-mediated hypersensitive response. HRAP protein does not bind to harpingₚₛₛ directly (Chen et al., “An Amphipathic Protein from Sweet Pepper can Dissociate Harpinₚₛₛ Multimeric Forms and Intensify the Harpinₚₛₛ-Mediated Hypersensitive Response,” Physiological & Molecular Pathology 52:139-49 (1998)). Using a fluorochrome tagged antibody to harpin to examine the interaction of harpingₚₛₛ and tobacco suspension cells, it was found that harpingₚₛₛ interacted with the cultured cells, but not with protoplasts with the cell walls being digested and removed. It was interpreted that harpingₚₛₛ was localized in the outer portion of the plant cell, probably on the cell well. However, it was not ruled out that the binding factor was located on the plasma membrane.

The present invention seeks to identify receptors for hypersensitive response elicitor proteins or polypeptides and uses of such receptors.

### SUMMARY OF THE INVENTION

The present invention is directed to an isolated protein which serves as a receptor in plants for a plant pathogen hypersensitive response elicitor. Also disclosed are nucleic acid molecules encoding such receptors as well as expression vectors, host cells, transgenic plants, and transgenic plant seeds containing such nucleic acid molecules.

The protein of the present invention can be used with a method of identifying agents targeting plant cells by forming a reaction mixture including the protein and a candidate agent, evaluating the reaction mixture for binding between the protein and the candidate agent, and identifying candidate compounds which bind to the protein in the reaction mixture as plant cell targeting agents.

The nucleic acid molecule of the present invention can be used in a method of identifying agents targeting plant cells by forming a reaction mixture including a cell transformed with the nucleic acid molecule of the present invention and a candidate agent, evaluating the reaction mixture for binding between protein produced by the host cell and candidate agent, and identifying candidate compounds which bind to the protein or the host cell in the reaction mixture as plant cell targeting agents.

Another aspect of the present invention relates to a method of enhancing a plant's receptivity to treatment with hypersensitive response elicitors by providing a transgenic plant or transgenic plant seed transformed with the nucleic acid molecule of the present invention.

The present invention is also directed to a method of imparting disease resistance, enhancing growth, controlling insects, and/or imparting stress resistance to plants by providing a transgenic plant or transgenic plant seed transformed with a DNA construct effective to silence expression of a nucleic acid molecule encoding a receptor in accordance with the present invention.

The discovery of the present invention has great significance. This putative receptor protein can be used as a novel way to screen for new inducers of plant resistance against insect, disease, and stress, and of growth enhancement. This protein is the first step toward the understanding of the harpin induced signal transduction pathway in plants. Further studies of this pathway will provide more possible targets for new plant vaccine and growth enhancement products development. In addition, this protein can serve as an anchor providing-a new way to target anything to the plant cells.

### BRIEF DESCRIPTION OF THE DRAWINGS

FIG. 1 shows a yeast two-hybrid screening with the Erwinia amylovora hypersensitive response elicitor (i.e. harpin) and a schematic representation of the interaction between harpin and a cDNA encoded polypeptide. Harpin is fused to a LexA protein which contains a DNA binding domain (“BD”). The cDNA encoded polypeptide is fused to the GAL4 transcription activation domain (“AD”). This interaction targets the activation domain to two different LexA-dependent promoters with consequent activation of the transcription of the HIS3 and lacZ reporter genes.

FIGS. 2A-B show that the Erwinia amylovora hypersensitive response elicitor (i.e. harpin) is a good yeast two-hybrid bait. Reporter genes were not expressed in yeast strain L40 containing plasmids expressing the LexA—harpin fusion in combination with plasmids expressing the GAL4 activation domain alone, or fused to unrelated protein. Therefore, harpin is not autoactive in this yeast two-hybrid system. In addition, reporter genes were not expressed in yeast strain L40 containing plasmids expressing the GAL4 activation domain-harpin fusion in combination with plasmids expressing LexA alone, or fused to unrelated protein. FIG. 2A shows a β-galactosidase assay where blue color indicates the expression of lacZ reporter gene. FIG. 2B shows a synthetic minimal (“SD”) media plate which lacks leucine, tryptophan, and histidine. Growth on such a plate indicates the expression of the HIS3 reporter gene.

FIGS. 3A-B show the interaction between AtHrBP1p (hypersensitive response elicitor binding protein 1) and a hypersensitive response elicitor (i.e. harpin) is specific. Reporter genes were expressed in yeast strain L40 containing plasmids expressing the GAL4 activation domain-AtHrBP1 fusion in combination with plasmids expressing LexA fused to hypersensitive response elicitor (i.e. harpin), but were not expressed in combination with LexA alone, or LexA fused to unrelated proteins. FIG. 3A is a β-galactosidase assay where the blue color indicates the expression of lacZ reporter gene. FIG. 3B is an SD media plate which lacks leucine, tryptophan, and histidine. Growth on such a plate indicates the expression of the HIS3 reporter gene.

FIGS. 4A-B show the interaction of HrBP1p and a hypersensitive response elicitor (i.e. harpin) in another orientation. Reporter genes were expressed in yeast strain L40 containing plasmids expressing the LexA-AtHrBP1p fusion in combination with plasmids expressing GAL4 activation domain fused to harpin, but were not expressed in combination with GAL4 activation domain alone, or GAL4 activation domain fused to unrelated proteins. Therefore, interaction between harpin and HrBP1p is specific. FIG. 4A shows a β-galactosidase assay where blue color indicates the expression of lacZ reporter gene. FIG. 4B shows an SD media plate which lacks leucine, tryptophan, and histidine. Growth on such a plate indicates the expression of the HIS3 reporter gene.

FIG. 5 shows the gene structure of AtHrBP1 and a schematic representation of the exons and introns of the AtHrBP1 gene. When comparing the AtHrBP1 cDNA sequence with the Arabidopsis thaliana genomic DNA sequence published in a public database, four exons and three introns were discovered.

FIG. 6 shows a Northern blot using RNA probe complementary to bases 651-855 of AtHrBP1 coding region (SEQ ID NO:29).

FIGS. 7A-B show that the interaction between rice HrBP1p (R6p) and harpin is specific. Reporter genes were expressed in yeast strain L40 containing plasmids expressing the GAL4 activation domain-rice HrBP1p fusion in combination with plasmids expressing LexA fused to harpin or harpin 137-180 amino acids, but were not expressed in combination with LexA alone, LexA fused to unrelated proteins, or fused to harpin 210-403 amino acids. FIG. 7A shows a β-galactosidase assay where blue color indicates the expression of the lacZ reporter gene. FIG. 7B shows an SD media plate, which lacks leucine, tryptophan, and histidine. Growth on such a plate indicates the expression of the HIS3 reporter gene.

FIGS. 8A-C show an alignment of HrBP1p amino acid sequences for the receptors from cotton (SEQ ID NO:6), soybean (SEQ ID NO:8), barley (SEQ ID NO:10), tomato (SEQ ID NO:12), rice (SEQ ID NO:14 and SEQ ID NO:16), potato (SEQ ID NO:18), wheat (SEQ ID NO:20 and SEQ ID NO:22), maize (SEQ ID NO:24), grapefruit (SEQ ID NO:26), apple (SEQ ID NO:28), tobacco (SEQ ID NO:30), grape (SEQ ID NO:32 and SEQ ID NO:34), and Arabidopsis thaliana (SEQ ID NO:1).

FIG. 9 shows a chart of the AtHrBP1p full-length and truncated polypeptides that were screened for their ability to interact with the harpin protein. The different HrBP1p fragments were utilized in the yeast-two hybrid system along with the harpin protein.

FIG. 10 shows the purified proteins used for in vitro binding studies. 1.2-1.5 μg of protein/lane was electrophoresed on a denaturing 10% polyacrylamide gel and stained with coomassie blue. Lane 1, standards; lane 2, HrpN; lane 3, HrBP1p; lane 4, TL-HrBP1p. The molecular masses of the standards are indicated on the left side of Lane 1.

FIGS. 11A-B show that AtHrBP1p interacts specifically with HrpN during affinity chromatography. Partially purified HrBP1p was mixed with HrpN-conjugated (HrpN) or mock-conjugated (C) agarose beads in binding buffer (20 mM Tris HCl, 50 mM NaCl, 0.2 mM EDTA, 1 mM DTT) and the beads were washed ten times with binding buffer (4 to 10 bed volumes each). Successive step elutions were done in binding buffer containing 200, 500, 750, 1000, and 1500 mM NaCl (2 bed volumes each). Selected fractions were run on denaturing 10% polyacryamide gels and the proteins were stained with silver. In FIG. 11A, the buffers contained no detergent. In FIG. 11B, the binding, wash, and elution buffers all contained 0.2% CHAPS. Horizontal arrows show the position of AtHrBP1p. The diagonal arrow points to HrpN. The molecular masses of the standards are indicated on the left side of each gel.

FIG. 12 shows the constructs used to “knockout” AtHrBP1 gene in Arabidopsis.

FIGS. 13A-C show a Pseudomonas syringae p.v. tomato DC3000 assay on wild type and AtHrBP1 “knockout” transgenic Arabidopsis plants. FIG. 13A is a picture taken 7 days after P. syringae inoculation. In FIG. 13B, leaf disks were harvested. Bacteria were extracted from leaf disks and plated onto King's B agar plate containing 100 μg rifampicin/ml. FIG. 13C shows the bacteria count from plates in FIG. 13B. The prefix “as” signifies an anti-sense line and “d” refers to a double-stranded RNA line.

FIGS. 14A-B show results from a study evaluating the differences in growth between wild type Arabidopsis thaliana and AtHrBP1 transgenic plant lines. There were 10 plant per line, except line 14-7, which had 9 plants. In FIG. 14A, the percentage of plants with 4 true leaves >1 mm in length was determined at sequential days after sowing. FIG. 14B shows the average diameter of maximum rosette radius of the plants when they entered the four-leaf stage. The standard deviation for each test group is indicated in the figure.

FIG. 15 shows wild type Arabidopsis thaliana and AtHrBP1 transgenic plant lines 32 days after sowing. Stems of the AtHrBP1 transgenic plants were more elongated than those of the wild type plants.

FIG. 16 shows the construct used to overexpress AtHrBP1 in tobacco.

FIGS. 17A-B show the height of wild type and AtHrBP1 overexpressing tobacco plants 52 days after they were transferred to soil. FIG. 17A is a picture taken 52 days after plants were transferred to soil. FIG. 17B shows average height of 8 plants per line.

FIGS. 18A-B show the results of a TMV assay on wild type and AtHrBP1 overexpressing tobacco plants. FIG. 18A is a picture taken 3 days after TMV inoculation. FIG. 18B shows the average virus lesion diameter from 5 plants per line 3 days after TMV inoculation.

FIG. 19 shows the 52-day-old wild type and two independent AtHrBP1p over-expressing Xanthi NN tobacco plants inoculated with Pseudomonas solanacearum, by root cutting.

### DETAILED DESCRIPTION OF THE INVENTION

The present invention is directed to isolated receptors for hypersensitive response elicitor proteins or polypeptides. Also disclosed are DNA molecules encoding such receptors as well as expression systems, host cells, and plants containing such molecules. Uses of the receptors themselves and the DNA molecules encoding them are disclosed. The receptor for a hypersensitive response elicitor from a plant pathogen can be from a monocot or a dicot.

One example of such a receptor is that found in Arabidopsis thaliana which has the amino acid sequence of SEQ ID NO:1 as follows:

```
Met Ala Thr Ser Ser Thr Phe Ser Ser Leu Leu Pro Ser Pro Pro Ala
    1 5 10 15
Leu Leu Ser Asp His Arg Ser Pro Pro Pro Ser Ile Arg Tyr Ser Phe
                          20 25 30
Ser Pro Leu Thr Thr Pro Lys Ser Ser Arg Leu Gly Phe Thr Val Pro
                  35 40 45
Glu Lys Arg Asn Leu Ala Ala Asn Ser Ser Leu Val Glu Val Ser Ile
          50 55 60
Gly Gly Glu Ser Asp Pro Pro Pro Ser Ser Ser Gly Ser Gly Gly Asp
  65 70 75 80
Asp Lys Gln Ile Ala Leu Leu Lys Leu Lys Leu Leu Ser Val Val Ser
                                  85 90 95
Gly Leu Asn Arg Gly Leu Val Ala Ser Val Asp Asp Leu Glu Arg Ala
                        100 105 110
Glu Val Ala Ala Lys Glu Leu Glu Thr Ala Gly Gly Pro Val Asp Leu
                115 120 125
Thr Asp Asp Leu Asp Lys Leu Gln Gly Lys Trp Arg Leu Leu Tyr Ser
        130 135 140
Ser Ala Phe Ser Ser Arg Ser Leu Gly Gly Ser Arg Pro Gly Leu Pro
145 150 155 160
Thr Gly Arg Leu Ile Pro Val Thr Leu Gly Gln Val Phe Gln Arg Ile
                                165 170 175
Asp Val Phe Ser Lys Asp Phe Asp Asn Ile Ala Glu Val Glu Leu Gly
                        180 185 190
Ala Pro Trp Pro Phe Pro Pro Leu Glu Ala Thr Ala Thr Leu Ala His
                195 200 205
Lys Phe Glu Leu Leu Gly Thr Cys Lys Ile Lys Ile Thr Phe Glu Lys
        210 215 220
Thr Thr Val Lys Thr Ser Gly Asn Leu Ser Gln Ile Pro Pro Phe Asp
225 230 235 240
Ile Pro Arg Leu Pro Asp Ser Phe Arg Pro Ser Ser Asn Pro Gly Thr
                                245 250 255
Gly Asp Phe Glu Val Thr Tyr Val Asp Asp Thr Met Arg Ile Thr Arg
                        260 265 270
Gly Asp Arg Gly Glu Leu Arg Val Phe Val Ile Ala
                275 280
```

This protein, known as AtHrBP1p, is encoded by a cDNA molecule having SEQ ID NO:2 as follows:

| | |
|---|---|
| tttttccttc tcaacaatgg cgacttcttc tactttctcg tcactactac cttcaccacc | 60 |
| agctcttctt tccgaccacc gttctcctcc accatccatc agatactcct tttctccctt | 120 |
| aactactcca aaatcgtctc gtttgggttt cactgtaccg gagaagagaa acctcgctgc | 180 |
| taattcgtct ctcgttgaag tatccattgg cggagaaagt gacccaccac catcatcatc | 240 |
| tggatcagga ggagacgaca agcaaattgc attactcaaa ctcaaattac ttagtgtagt | 300 |
| ttcgggatta aacagaggac ttgtggcgag tgttgatgat ttagaaagag ctgaagtggc | 360 |
| tgctaaagaa cttgaaactg ctgggggacc ggttgattta accgatgatc ttgataagct | 420 |
| tcaagggaaa tggaggctgt tgtatagtag tgcgttctct tctcggtctt taggtggtag | 480 |
| ccgtcctggt ctacctactg gacgtttgat ccctgttact cttggccagg tgtttcaacg | 540 |
| gattgatgtg tttagcaaag attttgataa catagcagag gtggaattag gagccccttg | 600 |
| gccatttccg ccattagaag ccactgcgac attggcacac aagtttgaac tcttaggcac | 660 |
| ttgcaagatc aagataacat ttgagaaaac aactgtgaag acatcgggaa acttgtcgca | 720 |
| gattcctccg tttgatatcc cgaggcttcc cgacagtttc agaccatcgt caaaccctgg | 780 |
| aactggggat ttcgaagtta cctatgttga tgataccatg cgcataactc gcggggacag | 840 |
| aggtgaactt agggtattcg tcattgctta attctcaaag ctttgacatg taaagataaa | 900 |
| taaatacttt ctgcttgatg cagtctcatg agttttgtac aaatcatgtg aacatataaa | 960 |
| tgcgctttat aagtaaatga gtgtcttgtt caatgaatca | 1000 |

The genomic DNA molecule containing the receptor encoding cDNA molecule of SEQ ID NO:2 has SEQ ID NO:3 as follows:

| | |
|---|---|
| aattagaaaa attaacaacc aacatctagt tagaatattt aatttgcacc aatgtcttcg | 60 |
| agtatagtga aaaaaataga agatcgaata tcgaatagta cgtatagaat catctagatc | 120 |
| cattcgaact aacgtctact tttcttttcc agcattaaca tgtagcttgt cattagcatt | 180 |
| tacatgttgc aaataacaca aattgggaaa ttgaaagact aaaaaacctt gtacagcaga | 240 |
| tggtttaaca cgtggattca tggacacaaa cagaaaacgg cagaactaag cacaaaaacg | 300 |
| tcaactaagc atatcaaagc ttttaatgca agcctaatat aaacacaagt ggttatccat | 360 |
| aatctgttct taatctcttg cagtagttat cttttcatta ttcgcaattc gcaattctat | 420 |
| attcttatat ttcaacttgt tcttcttcca aattgtaatt atatctacat cgtcttagct | 480 |
| tgaccattat agctccagta ccaagttctc ttcttaactt taatatcagc tactattctc | 540 |
| atactgtaaa tatcttttgt tcaccaaaca tatatttcga accaaactgc taaaagctta | 600 |
| tcataaattg cagttctagc cacacaattt tgcagttcca accattaaat gccacaaaat | 660 |
| ttggacgatt tcttaagaca agaagaacat agcaaccaaa ccttattgat taaatatgaa | 720 |
| atgtctccat aaaactggga gatttcccca aataaagaga acacggcaaa tgttcacgta | 780 |
| atctccaaga tgaatgttta attttttctt tcagaaaaaa acaaaaaaac ttaactcaat | 840 |
| atagacaact agaatggata ccaactaagc aaaagaaatt caaaagacaa atatatattg | 900 |
| gatatgaagt tacattattt tcaaacttta tatactacta aaagcctaaa aatttgttct | 960 |
| aaaatgatat ccaaataaat ggaaggcatg aatgtcatat gactaaaaga gaaaaacaca | 1020 |
| cctgtatata agtattggat catgctgcct ccgagtgaca aaacatacga tgtgggtctt | 1080 |
| tattgggcca tacttaaatg gaaaaaggag aaaaaaaatt gggcaatgtc tatggtcgaa | 1140 |
| atttatatgt tttacatcaa taaaatcaat atttaatttt atatatgtgg gtcttaatct | 1200 |
| agtattatct acatagatta aaatcaaagt actgcatatg gtccataata atacaaccaa | 1260 |
| agcaaattaa aattttgtgg cacaaaacga catcatttta ctcagaaagt aatatgcaat | 1320 |
| ttcgtttacg cacacacgta tacgcgctaa taacccgtgg tgcttctcaa atcacataat | 1380 |
| aattaaagtc ttcttcttct tcttcttctc tacaaattat ctcactctct tcgttttttt | 1440 |
| ttccttctca acaatggcga cttcttctac tttctcgtca ctactacctt caccaccagc | 1500 |
| tcttctttcc gaccaccgtt ctcctccacc atccatcaga tactcctttt ctcccttaac | 1560 |
| tactccaaaa tcgtctcgtt tgggtttcac tgtaccggag aagagaaacc tcgctgctaa | 1620 |
| ttcgtctctc gttgaagtat ccattggcgg agaaagtgac ccaccaccat catcatctgg | 1680 |
| atcaggagga gacgacaagc aaattgcatt actcaaactc aaattacttg tgagtctgat | 1740 |
| tcaaaccaat cggtgaaatt ataagaaatt ggtttcgttt ctttggaatt agggtttata | 1800 |
| ttactgttaa gattcgatta tagagtgaat tttgggaaga tttttcagat ttgatttgtg | 1860 |
| atgtgttgtg ttgtgagaaa ttgcagagtg tagtttcggg attaaacaga ggacttgtgg | 1920 |
| cgagtgttga tgatttagaa agagctgaag tggctgctaa agaacttgaa actgctgggg | 1980 |
| gaccggttga tttaaccgat gatcttgata agcttcaagg gaaatggagg ctgttgtata | 2040 |
| gtagtgcgtt ctcttctcgg tctttaggtg gtagccgtcc tggtctacct actggacgtt | 2100 |
| tgatccctgt tactcttggc caggtaattc ttgaatcatt gctctgtttt tacccgtcaa | 2160 |
| gattcggttt ttcgggtaag ttgttgagga gtttatgtgc atggtctagt ctatcatcaa | 2220 |
| tagtcttgct tgagtttgaa tggggctgag ctaagaatct agctttctga ggttacaatt | 2280 |
| tggtaatgtc atcttatact cgaaagcaaa cttttttcta tattgtcaag tttatgtgta | 2340 |
| cggtctggtc tatcattggt agtctttgtt gagcttgaat ggtgaggagc ttagaatcta | 2400 |
| gcaatgtcat ctactcctta atcatttttt tctatattgc caagtttatg tgtacggtct | 2460 |
| tagtcaatca tctttattct tggttgagtt tgaatggtga tgagcttaga atctagcttt | 2520 |
| ctttggttta aatttggcaa agaaccatac ctgaatcggt agaaagcaaa cttctttaat | 2580 |
| attatctctt gtttctgaat cattaaaaca ggtgtttcaa cggattgatg tgtttagcaa | 2640 |
| agattttgat aacatagcag aggtggaatt aggagcccct tggccatttc cgccattaga | 2700 |
| agccactgcg acattggcac acaagtttga actcttaggt ttgcatttcc ctttctctca | 2760 |
| ttaagtttat cgaattgtgt aagagcaaaa taacttatct gtatctttga catttatggg | 2820 |
| gaaaacaggc acttgcaaga tcaagataac atttgagaaa acaactgtga agacatcggg | 2880 |
| aaacttgtcg cagattcctc cgtttgatat cccgaggctt cccgacagtt tcagaccatc | 2940 |
| gtcaaaccct ggaactgggg atttcgaagt tacctatgtt gatgatacca tgcgcataac | 3000 |
| tcgcggggac agaggtgaac ttagggtatt cgtcattgct taattctcaa agctttgaca | 3060 |
| tgtaaagata aataaatact ttctgcttga tgcagtctca tgagttttgt acaaatcatg | 3120 |
| tgaacatata aatgcgcttt ataagtaaat gagtgtcttg ttcaatgaat catatgaaag | 3180 |
| aatttgtatg actcagaaaa ttggacaatg atatagacct tccaaatttt gcaccctcta | 3240 |
| atgtgagata ttagtgattt tttcttaggt tggtagagag aacggattgg caaaaaaata | 3300 |
| tcgaaggtca atgattaaca gcaaaaccat atcttgatga ttcaaaatat agagttaaca | 3360 |
| agcaaagatg agacaatctt atacgagaga gctaaaacaa atggattcca aatccagcaa | 3420 |
| gtacaaaaat cgcagaaaat aagatgaaac caacttaaaa cagagatgtt ccctttccct | 3480 |
| tcttgtcacc accgatctcg aaatgcttgc acctctgaaa taaacaacaa accaacacaa | 3540 |
| tgtaagcaaa ttaccaagtt acaaatccgg tataatgaac tgatctatgt tctatgcacc | 3600 |
| ttgataggac gctgcgaaaa gtgcttgcag ctttgacact gaagcctcaa aacaatcttc | 3660 |
| ttcgtggtct tagcctgtta acaagattca caagatgtat ctcagtccaa aactgagact | 3720 |
| attggaatgt ctgtttcctc acagctcact tccaaaattc tactataaat ggttccttaa | 3780 |
| aactacctca tttcaactaa ctagacctaa ttcaaactga aaaaacaatc aatgcatgat | 3840 |
| aatcaatgtt acctttttgt ggaagacagg cttagtctga ccaccataac cagattgttt | 3900 |
| acggtcataa cgacgctttc cttgagcagc aagactgtct ttacccttct tgtattgggt | 3960 |
| aaccttgtgc aaagtatgct ttttgcattc cttgttctta cagtaagtgt tctttgtctt | 4020 |
| tggaatgttc accttcaaaa ttcataaaat caaaaatgaa tcactcacac acatacaaaa | 4080 |
| tcaagagact tttaaggtta atcaaaatac aaacatcatt tagattgaaa acttttatga | 4140 |
| tagatctgaa aaacaataca ataaatcaat caaccatgta ttgttgttct tcaaagtcaa | 4200 |
| cgaactttac aaattccaaa atcacatcga aagagaagaa acaatttacc attttcgcgt | 4260 |

Another example of a receptor in accordance with the present invention is that found in rice which has a partial amino acid sequence of SEQ ID NO:4 as follows:

```
Val Ala Ala Leu Lys Val Lys Leu Leu Ser Ala Val Ser Gly Leu Asn
    1 5 10 15
Arg Gly Leu Ala Gly Ser Gln Glu Asp Leu Asp Arg Ala Asp Ala Ala
                          20 25 30
Ala Arg Glu Leu Glu Ala Ala Ala Gly Gly Gly Pro Val Asp Leu Glu
                  35 40 45
Arg Asp Val Asp Lys Leu Gln Gly Arg Trp Arg Leu Val Tyr Ser Ser
          50 55 60
Ala Phe Ser Ser Arg Thr Leu Gly Gly Ser Arg Pro Gly Pro Pro Thr
  65 70 75 80
Gly Arg Leu Leu Pro Ile Thr Leu Gly Gln Val Phe Gln Arg Ile Asp
                                  85 90 95
Val Val Ser Lys Asp Phe Asp Asn Ile Val Asp Val Glu Leu Gly Ala
                        100 105 110
Pro Trp Pro Leu Pro Pro Val Glu Leu Thr Ala Thr Leu Ala His Lys
                115 120 125
Phe Glu Ile Ile Gly Thr Ser Ser Ile Lys Ile Thr Phe Asp Lys Thr
        130 135 140
Thr Val Lys Thr Lys Gly Asn Leu Ser Gln Leu Pro Pro Leu Glu Val
145 150 155 160
Pro Arg Ile Pro Asp Asn Leu Arg Pro Pro Ser Asn Thr Gly Ser Gly
                                165 170 175
Glu Phe Glu Val Thr Tyr Leu Asp Gly Asp Thr Arg Ile Thr Arg Gly
                        180 185 190
Asp Arg Gly Glu Leu Arg Val Phe Val Ile Ser
                195 200
```

This protein, known as R6p, is encoded by a cDNA molecule which has a partial sequence corresponding to SEQ ID NO:5 as follows:

| | |
|---|---|
| cgtggctgcg ctcaaagtca agcttctgag cgcggtgtcc gggctgaacc gcggcctcgc | 60 |
| ggggagccag gaggatcttg accgcgccga cgcggcggcg cgggagctcg aggcggcggc | 120 |
| gggtggcggc cccgtcgacc tggagaggga cgtggacaag ctgcaggggc ggtggaggct | 180 |
| ggtgtacagc agcgcgttct cgtcgcggac gctcggcggc agccgccccg gcccgcccac | 240 |
| cggccgcctc ctccccatca ccctcgggca ggtgtttcag aggatcgatg ttgtcagcaa | 300 |
| ggacttcgac aacatcgtcg atgtcgagct cggcgcgcca tggccgctgc cgccggtgga | 360 |
| gctgacggcg accctggctc acaagtttga gatcatcggc acctcgagca taaagatcac | 420 |
| attcgacaag acgacggtga agacgaaggg gaacctgtcc cagctgccgc cgctggaggt | 480 |
| ccctcgcatc ccggacaacc tccggccgcc gtccaacacc ggcagcggcg agttcgaggt | 540 |
| gacctacctc gacggcgaca cccgcatcac ccgcggggac agaggggagc tcagggtgtt | 600 |
| cgtcatctcg tga | 613 |

Another example of a receptor in accordance with the present invention is found in cotton and has the amino acid sequence of SEQ ID NO:6 as follows:

MASSSFLLESPASIFSSSSIKAHLYLPKPYPFIVSVKRRRSERKRNPVLK
SAVGDVSVVDTPPPPPPPPQDAKSELISSLKLKLLGIVSGLNRGLAANQD
DLGKADDAAKELETVAGPVDLLTDLDKLQGRWKLIYSSAFSSRTLGGSRP
GLPTGRLLPVTLGQVFQRIDVISKDFDNIAEIELGAPWPLPPLEVTATLA
HKFEIIGSSKIKITFEKTSVKTRGTFSQLPSLDVPRIPDALRPPSNPGSG
DFDVTFIDADTRITRGDRGELRVFVIS

This protein, known as GhHrBP1p, is encoded by a cDNA molecule which has a partial sequence corresponding to SEQ ID NO:7 as follows:

AAAGCTTTCTTGCAAAAAGCTCCGAAAAAGGGCCAGCAAAAGCCACTTGA
GAGCCAATGGCTTCTTCAAGTTTTCTTCTAGAATCTCCGGCGTCTATCTT
CTCTTCTTCCTCCATTAAAGCTCATCTCTATCTCCCGAAACCCTACCCTT
TTATTGTTAGCGTGAAACGGCGCCGTTCGGAAAGGAAGCGAAACCCTGTT
TTAAAATCGGCTGTTGGAGATGTCTCCGTCGTTGACACCCCACCGCCGCC
GCCGCCTCCACCTCAAGATGCTAAATCTGAACTCATTTCTTCTTTGAAGC
TTAAATTACTGGGTATTGTTTCTGGGCTGAATAGAGGTCTTGCTGCGAAC
CAAGATGATCTCGGAAAAGCAGATGATGCCGCCAAGGAACTCGAAACGGT
TGCTGGACCTGTGGACTTATTGACCGATCTTGATAAGCTGCAAGGGAGAT
GGAAACTGATATACAGCAGTGCATTCTCGTCTCGTACACTCGGCGGGAGC
CGTCCTGGACTTCCCACTGGAAGGTTGCTCCCTGTAACTCTCGGCCAGGT
TTTTCAGAGAATTGATGTCATAAGCAAAGATTTTGATAATATAGCAGAAA
TTGAATTGGGAGCTCCATGGCCATTACCTCCACTTGAAGTTACTGCTACC
TTAGCTCACAAATTTGAAATCATAGGATCTTCAAAGATCAAAATAACATT
CGAGAAAACGAGTGTGAAAACTAGAGGGACCTTTTCTCAGCTTCCGTCAT
TGGATGTACCTCGGATTCCCGACGCTTTGAGGCCTCCATCTAATCCAGGG
AGCGGCGACTTTGATGTTACCTTCATTGATGCCGATACCCGAATCACCAG
AGGAGATAGAGGTGAGCTTAGGGTTTTTGTCATCTCATAAATTAGTAAGC
ACATCTAATATCAAAGCTCGTATGCACTCTCATTACTTCATATATTGTCT
GTATGTGTATATATCATTGGGGGTGATCCGTAACTTTTTGTAGAATTAAT
ATTTTAATGTAATTACGAATATTATGTATGTAAATTTTCGAATCAATTTA
ATAGTTTAATCGTG

Another example of a receptor in accordance with the present invention is found in soybean and has the amino acid sequence of SEQ ID NO:8 as follows:

MASLNLLPHPPLFSSFLHRPHCNTHLLLTPKPSQRRPSLVVKSTVGVADP
SPSSSSYAGDTSDSISSLKLNLLSAVSGLNRGLAASEDDLRKADDAAKEL
EAAGGLVDLSLGLDNLQGRWKLIYSSAFSSRTLGGSRPGPPIGRLLPITL
GQVFQRIDILSKDFDNIVELQLGAPWPLPPLEATATLAHKFELIGSSKIK
IVFEKTTVKTAGNLSQLPPLEVPRIPDALRPPSNTGSGEFEVTYLDSDTR
ITRGDRGELRVFVIA

This protein, known as GmHrBP1p, is encoded by a cDNA molecule which has a partial sequence corresponding to SEQ ID NO:9 as follows:

GGCACGAGGCTCCAATCCATGGCTTCCCTGAACCTCCTTCCCCACCCTCC
ACTTTTCTCTTCTTTCCTTCACAGACCACACTGCAACACCCATCTTCTTC
TCACACCAAAACCTTCTCAACGAAGGCCTTCTCTTGTGGTCAAATCTACT
GTGGGTGTGGCTGACCCTTCTCCATCTTCTTCTTCCTACGCTGGGGATAC
CTCTGATTCCATCTCTTCTTTGAAGCTCAATCTGCTGAGTGCTGTTTCTG
GGCTAAATAGAGGCCTTGCTGCAAGCGAAGACGATCTTCGAAAGGCAGAT
GATGCTGCTAAGGAACTTGAAGCTGCTGGAGGACTTGTGGATCTCTCGCT
TGGTCTTGACAATTTGCAAGGAAGATGGAAACTCATTTATAGCAGCGCAT
TTTCGTCTCGAACCCTTGGTGGAAGCCGTCCTGGTCCTCCCATAGGAAGA
CTCCTTCCTATTACTCTTGGACAGGTTTTTCAACGAATTGACATCTTGAG
CAAAGATTTTGATAACATAGTGGAGCTTCAACTAGGTGCTCCATGGCCCC
TACCACCCCTTGAAGCGACTGCCACATTAGCTCACAAATTTGAACTCATA
GGATCTTCAAAGATAAAGATAGTATTTGAGAAAACCACTGTGAAGACAGC
TGGGAATTTGTCACAGTTGCCACCATTGGAGGTGCCTCGGATTCCCGATG
CATTGAGGCCTCCATCTAATACGGGAAGCGGTGAATTTGAAGTTACATAT
CTTGACTCGGATACTCGCATCACAAGAGGAGACAGAGGCGAGCTAAGGGT
CTTTGTGATTGCTTGAGTTCCTGGTGAATGCAACTATGCACTATGCATTT
TCTCTGTTGGACTTAAAAAAAAAAGGTTTCAACACCTTGTGCCATCATTT
TGTTTAGTTTTTTCCTCCTGATGGTATTTGTTCTAAGTTCTTCAATATTG
TAAACATGATGGAATTAAACTCTACTATATAGTTCCAAGGAAGCAGGGTA
CTTTTTGTTTAAGTGTAACATATTTCTTTTTTAAGGAATAATTGCTTACA
GATCATTAGATATGGATACTTGAAT

Another example of a receptor in accordance with the present invention is found in barley and has the amino acid sequence of SEQ ID NO:10 as follows:

MAMASPSWSSCCTSTSTHSLPGPPASSKGRNPWRASSGRRSASGGKRQQK
LSIRAVAAPSAAVDYSDTGAGAGDIPSKIKLLSAVAGLNRGLAASQEDLD
RADAAARQLEAAAPAPVDLAKDLDKLQGRWRLVYSSAFSSRTLGGSRPGP
PTGRLLPITLGQVFQRIDVVSQDFDNIVELELGAPWPLPPVEATATLAHK
FEITGIASIKINFDKTTVKTNGNLSQLPLLEVPRIPDSLRPPTSNTGSGE
FNVTYLDDDTRITRGDRGELRVFVVT

This protein, known as HvHrBP1p, is encoded by a cDNA molecule which has a partial sequence corresponding to SEQ ID NO:11 as follows:

GCCGGTCGGCACCCAACTGGAGGTTCAGTTTCCTCGTTGCTCTCCTCCAT
TGATTGACCGCCTCCTTCCCTGAGGCGCACGGTACACGGACGGCACCCAT
GGCCATGGCATCGCCGTCGTGGTCATCCTGCTGCACCTCAACCTCCACCC
ATTCTCTGCCCGGTCCTCCCGCGAGCAGCCAGGGCAGGAACCCGTGGCGG
GCAAGCAGCGGCAGGAGGAGCGCCAGCGGAGGGAAGAGGCAGCAGAAGCT
GTCCATCCGCGCGGTGGCCGCACCGTCGGCCGCGGTGGACTACTCGGACA
CCGGCGCCGGCGCCGGCGACATCCCCTCGCTGAAAATCAAGCTGCCGAGC
GCCGTCGCCGGGCTGAACCGGGGCCTCGCTGCGAGCCAGGAGGACCTGGA
CCGGGCGGACGCGGCGGCGCGGCAGCTCGAGGCGGCGGCGCCGGCCCCCG
TGGACCTCGCCAAGGATCTCGACAAGCTGCAGGGGCGGTGGAGGCTGGTC
TACAGCAGCGCCTTCTCGTCGCGGACGCTCGGCGGCAGCCGCCCCGGCCC
GCCCACCGGTCGCCTCCTCCCCATCACCCTCGGCCAGGTGTTCCAGAGGA
TCGACGTGGTGAGCCAGGACTTCGACAACATCGTGGAGCTCGAGCTCGGC
GCCCCGTGGCCGCTGCCGCCGGTGGAGGCCACGGCCACGCTGGCACACAA
GTTTGAGATCACCGGAATCGCGAGTATCAAGATCAATTTCGACAAGACGA
CGGTGAAGACGAACGGGAACCTGTCCCAGCTGCCGCTGCTGGAGGTGCCC
CGCATCCCGGATAGCCTCAGGCCGCCGACTTCCAACACCGGGAGCGGCGA
GTTCAACGTGACCTATCTCGACGACGACACCCGCATCACCCGAGGGGACA
GGGGGGAGCTCAGGGTGTTCGTCGTCACATGAGCTTTTTTTTGCTGCGAT
CTCTCTCTTTGTAGTGCTCCAACTTTTTTTGGCCCGTAAAACAAGAGTCT
TGTACTAGTTCTATATATGCCTTTTGTTTTGGGGTTCACCCGTCCATCCG
CGGGAAACATCTATCGTGACGACTGTTCGATGTATAAGCGGAGTCGTCCG
ATTTACGCGGTTCCGTCGTCTTTTCGAAC

Another example of a receptor in accordance with the present invention is found in tomato and has the amino acid sequence of SEQ ID NO:12 as follows:

MASLLHSRLPLSRNHSLSNSCQSFPCHLPGRSKRSTQRLLEERSYDSKRS
LVCQSGIDEVTFIEPPGSKEAEAELIGSLKLKLLSAVSGLNRGLAASEDD
LKKADEAAKELESCAGAVDLAADLDKLQGRWKLIYSSAFSSRTLGGSRPG
PPTGRLLPITLGQVFQRIDVLSKDPDNIVELELGAPWPFPPVEATATLAH
KFELIGSSTIKIIFEKTTVKTTGNLSQLPPLEVPRIPDQFRPPSNTGSGE
FEVTYIDSDTRVTRGDRGELRVFVIS

This protein, known as LeHrBP1p, is encoded by a cDNA molecule which has a partial sequence corresponding to SEQ ID NO:13 as follows:

TCGATCCTTTTTCTGAAATTCAAGCTCAACCATGGCTTCTCTACTTCATT
CGAGACTTCCCCTTTCTCACAATCATTCTTTATCAAATTCTTGCCAATCT
TTCCCATGTCATCTCCCAGGAAGAAGCAAGAGAAGTACTCAAAGATTATT
AGAGGAAAGGAGCTATGACAGCAAGAGAAGTTTAGTTTGCCAGTCGGGTA
TTGATGAAGTCACTTTTATTGAGCCACCTGGTAGTAAAGAAGCTGAAGCG
GAGCTTATTGGGTCTCTCAAACTCAAGTTATTGAGTGCTGTTTCTGGGCT
AAACAGAGGTCTTGCTGCAAGTGAAGATGATCTAAAGAAGGCGGATGAGG
CTGCCAAGGAGCTAGAATCTTGTGCAGGAGCTGTAGATCTCGCAGCTGAT
CTTGATAAACTTCAAGGGAGGTGGAAATTGATATACAGCAGTGCATTCTC
ATCTCGTACTCTTGGTGGAAGTCGTCCTGGACCCCCCACTGGAAGACTTC
TTCCCATCACTCTTGGTCAGGTATTTCAAAGAATCGATGTACTGAGCAAA
GATTTTGACAACATAGTGGAGCTTGAATTAGGTGCTCCGTGGCCTTTCCC
GCCTGTTGAAGCAACTGCCACTTTAGCCCACAAATTTGAACTTATAGGAT
CATCTACGATTAAGATTATATTCGAGAAAACTACAGTGAAGACAACTGGA
AATTTATCACAGCTCCCACCATTAGAAGTGCCTCGCATACCAGATCAGTT
CAGGCCACCATCAAATACAGGAAGTGGTGAGTTTGAAGTTACCTACATCG
ATTCTGATACACGAGTAACAAGGGGAGACAGAGGAGAGCTTAGAGTTTTC
GTTATCTCATAAGTTAAGCTGCAATGAATATAGTCTTCCTACAATGTTTT
GTTGCTACAATTTCATGTAACAACATATCAAATGTGTAGATATGCTCAAC
ATTATTCTGCTGGTCACAGCTATCAAATCTGTAATGCTACTGCAAATTCA
AATCTGTATACAGTAAATTTGACATC

Another example of a receptor in accordance with the present invention is found in rice and has the amino acid sequence of SEQ ID NO:14 as follows:

MAAAVASSCCASTSARPLVRRAGSRNGKLWWAGGVRKARLLSISATAAAP
SGVDYAAGTGAAADDDAVAALKVKLLSAVSGLNRGLAGSQEDLDRADAAA
RELEAAAGGGPVDLERDVDKLQGRWRLVYSSAFSSRTLGGSRPGPPTGRL
LPITLGQVFQRIDVVSKDFDNIVDVELGAPWPLPPVELTATLAHKFEIIG
TSSIKITFDKTTVKTKGNLSQLPPLEVPRIPDNLRPPSNTGSGEFEVTYL
DGDTRITRGDRGELRVFVIS

This protein, known as OsHrBP1-1p, is encoded by a cDNA molecule which has a partial sequence corresponding to SEQ ID NO:15 as follows:

TCGCCATTGATTTTCTCTGTCTGCTCTGCTGCTCGCTTGCTTGCGCTGTC
CGGTTTAGCTCTGTCTAGCTAGGTAGACTGCGGCCATGGCGGCGGCGGTG
GCGTCGTCTTGCTGCGCCTCGACCAGCGCTCGCCCACTGGTTCGCCGCGC
CGGGAGCAGGAACGGGAAGCTGTGGTGGGCGGGTGGTGTCAGGAAGGCGC
GGCTGCTGTCCATCTCCGCCACGGCCGCGGCGCCGTCGGGCGTGGACTAC
GCGGCGGGCACCGGCGCCGCCGCCGACGACGACGCCGTGGCTGCGCTCAA
AGTCAAGCTTCTGAGCGCGGTGTCCGGGCTGAACCGCGGCCTCGCGGGGA
GCCAGGAGGATCTTGACCGCGCCGACGCGGCGGCGCGGGAGCTCGAGGCG
GCGGCGGGTGGCGGCCCCGTCGACCTGGAGAGGGACGTGGACAAGCTGCA
GGGGCGGTGGAGGCTGGTGTACAGCAGCGCGTTCTCGTCGCGGACGCTCG
GCGGCAGCCGCCCCGGCCCGCCCACCGGCCGCCTCCTCCCCATCACCCTC
GGGCAGGTGTTTCAGAGGATCGATGTTGTCAGCAAGGACTTCGACAACAT
CGTCGATGTCGAGCTCGGCGCGCCATGGCCGCTGCCGCCGGTGGAGCTGA
CGGCGACCCTGGCTCACAAGTTTGAGATCATCGGCACCTCGAGCATAAAG
ATCACATTCGACAAGACGACGGTGAAGACGAAGGGGAACCTGTCCCAGCT
GCCGCCGCTGGAGGTCCCTCGCATCCCGGACAACCTCCGGCCGCCGTCCA
ACACCGGCAGCGGCGAGTTCGAGGTGACCTACCTCGACGGCGACACCCGC
ATCACCCGCGGGGACAGAGGGGAGCTCAGGGTGTTCGTCATCTCGTGATC
GGACGGACGCGTTCGCGACATAGGTATGCGGCTTGCGATTCTGAAACTGA
AACTGAAGCGCACACACGGTTTTGTGTTCTTTCTCTGCTACTAGTAGATC
CTCACTCTCTTGATCTGACCATCTTTGTACTATACTTCAGTATTGTTCGT
GCGTTCTGTATTGTTATAGATTTTGCAGATATTCAACAAGTAGAGGGAAA
TATGTCAAAATGAGAAATCGAGG

Another example of a receptor in accordance with the present invention is found in rice and has the amino acid sequence of SEQ ID NO:16 as follows:

MAAAVASSCCASTSARPLVRRAGSRSGKLWWAGGGRKARLLSISATAAAP
SGVDYAAGTGAADDDAVAALKVKLLSAVSGLNRGLAASQEDLDRADAAAR
ELEAAAGGGPVDLEGDMDKLQGRWRLVYSSAFSSRTLGGSRPGPPTGRLL
PITLGQVFQRIDVVSKDFDNIVDVELGAPWPLPPVELTATLAHKFEIIGT
SSIKITFDKTTVKTKGNLSQLPPLEVPRIPDNLRPPSNTGSGEFEVTYLD
GDTRITRGDRGELRVFVIS

This protein, known as OsHrBP1-2p, is encoded by a cDNA molecule which has a partial sequence corresponding to SEQ ID NO:17 as follows:

TCGCCATTGATTTTCTCTGTCTGCTCTGCTGCTCGCTTGCTTGCGCTGTC
CGGTTTAGCTCTGTCTAGCTAGGTAGACTGGCGGCCATGGCGGCGGCGGT
GGCGTCGTCTTGCTGCGCCTCGACCAGCGCTCGCCCACTGGTTCGCCGCG
CCGGGAGCAGGAGCGGGAAGCTGTGGTGGGCGGGTGGTGGGAGGAAGGCG
CGGCTGCTGTCCATCTCCGCCACGGCCGCGGCGCCGTCGGGCGTGGACTA
CGCGGCGGGCACCGGCGCCGCCGACGACGACGCCGTGGCTGCGCTCAAAG
TCAAGCTTCTGAGCGCGGTGTCCGGGCTGAACCGCGGCCTCGCGGCGAGC
CAGGAGGATCTTGACCGGGCCGACGCGGCGGCGCGGGAGCTCGAGGCGGC
GGCGGGCGGCGGGCCCGTCGACCTGGAGGGGGACATGGACAAGCTGCAGG
GGCGGTGGAGGCTGGTGTACAGCAGCGCGTTCTCGTCGCGGACGCTCGGC
GGCAGCCGCCCCGGCCCGCCCACCGGCCGCCTCCTCCCCATCACCCTCGG
CCAGGTGTTTCAGAGGATCGATGTTGTCAGCAAGGACTTCGACAACATCG
TCGATGTCGAGCTCGGCGCGCCATGGCCGCTGCCGCCGGTGGAGCTGACG
GCGACGCTGGCTCACAAGTTTGAGATCATCGGCACCTCGAGCATAAAGAT
CACATTCGACAAGACGACGGTGAAGACGAAGGGGAACCTGTCCCAGCTGC
CGCCGCTGGAGGTCCCTCGCATCCCGGACAACCTCCGGCCGCCGTCCAAC
ACCGGCAGCGGCGAGTTCGAGGTGACCTACCTCGACGGCGACACCCGCAT
CACCCGCGGGGACAGAGGGGAGCTCAGGGTGTTCGTCATCTCGTGATCGG
ACGGACGCGTTCGCGACATAGGTATGCGGCTTGCGATTCTGAAACTGAAA
CTGAAGCGCACACACGGTTTTGTGTTCTTTCTCTGCTACTAGTAGATCCT
CACTCTCTTGATCTGACCATCTTTGTACTATACTTCAGTATTGTTCGTGC
GTTCTGTATTGTTATAGATTTTGCAGATATTCAACAAGTAGAGGGAAATA
TGCCAAAATGAG

Another example of a receptor in accordance with the present invention is found in potato and has the amino acid sequence of SEQ ID NO:18 as follows:

MASLLHSRLPLSHNHSLSNSCQSFPCHLPGRSKRSTQRFFEERSYDSKRA
LICQSGIDEVTFRLPGSKEAKAELIGSLKLKLLSAVSGLNRGLAASEDDL
KKADEAAKELESCAGAVDLAADLDKLQGRWKLIYSSAFSSRTLGGSRPGP
PTGRLLPITLGQVFQRIDVLSKDFDNIVELELGAPWPFPPVEATATLAHK
FELIGSSTIKIVFEKTTVKTTGNLSQLPPIEVPRIPDQFRPPSNTGNGEF
EVTYIDSDTRVTRGDRGELRVFVIS

This protein, known as StHrBP1p, is encoded by a cDNA molecule which has a partial sequence corresponding to SEQ ID NO:19 as follows:

CTACCACCAATCAAACTCCACAAAAGATCGATCCTTTTTCTGAAATTCAA
GCTCAACCATGGCTTCTCTACTTCATTCTAGACTTCCCCTTTCTCACAAT
CATTCTTTATCAAATTCTTGCCAATCTTTCCCCTGTCATCTCCCAGGAAG
AAGCAAGAGAAGTACTCAAAGATTCTTTGAGGAAAGGAGCTATGATAGCA
AGAGAGCCTTAATTTGTCAGTCGGGTATTGATGAAGTCACTTTTAGGCTA
CCTGGTAGTAAAGAAGCTAAAGCTGAGCTTATTGGGTCTCTCAAACTCAA
GTTATTGAGTGCTGTTTCTGGGCTAAACAGAGGTCTTGCTGCAAGTGAAG
ATGATCTAAAGAAGGCGGATGAGGCTGCCAAGGAGCTGGAATCTTGTGCA
GGAGCTGTAGATCTCGCAGCTGATCTTGATAAGCTTCAAGGGAGGTGGAA
ATTGATATACAGCAGTGCATTCTCATCTCGTACTCTTGGTGGAAGTCGTC
CTGGCCCCCCCACTGGAAGACTTCTTCCCATCACTCTTGGTCAGGTATTT
CAAAGAATTGATGTACTAAGCAAGGATTTTGACAACATAGTGGAGCTTGA
ATTAGGTGCTCCGTGGCCTTTCCCACCTGTTGAAGCAACTGCCACTTTAG
CCCACAAATTTGAACTTATAGGATCATCTACAATTAAGATTGTATTCGAA
AAACTCAGTGAAGACAACTGGAAATTTATCACAGTTGCCACCAATAGAAG
TGCCTCCATACCAGATCAGTTCAGGCCACCATCAAATACAGGAAATGGTG
AGTTTGAAGTTACCTATATCGATTCTGATACACGTGTAACAAGGGGAGAC
AGAGGAGAGCTTAGAGTTTTCGTTATCTCATAAGTTAAGCTGCAATAAAT
ATAGTTTTCCTACAATATTTTGTTGCTACAATTTCATGTAACAACATATC
NAATGTATAGATATGCTCAACATTATTCTGCTGGTCAAAGCTAGCAAATT
TGTAATGCTACTGCAAATTCAAATCTGTATACAGTAAATTTGACATGTGA
TGGAGTTATGCAGTGAGATTTCNANAAT

Another example of a receptor in accordance with the present invention is found in wheat and has the amino acid sequence of SEQ ID NO:20 as follows:

MAMASPSWSSCCASTSTRPLPSPPASSKSRNPWRASSGRRSASGGKRRQQ
LSIRAVAAPSSAVDYSDTAAGAGDVPSLKIKLLSAVAGLNRGLAASQEDL
DRADAAARQLEAAAPAPVDLAKDLDKLQGRWRLVYSSAFSSRTLGGSRPG
PPTGRLLPITLGQVFQRIDVVSQDFDNIVELELGAPWPLPPVEATATLAH
KFEITGIASIKINFDKTTVKTKGNLSQLPLLEVPRIPDSLRPTTSNTGSG
EFDVTYLDDGTRITRGDRGELRVFVVS

This protein, known as TaHrBP1-1p, is encoded by a cDNA molecule which has a partial sequence corresponding to SEQ ID NO:21 as follows:

GAATTCGGCACGAGCTGACCTCTTGCCGGTCGGCGCCCAATTGAAAATTT
CTTTTCTTTTTGCTCTCCTGATCGATTGACTGCCTCACGGACGGTGCCCA
TGGCCATGGCATCGCCGTCGTGGTCATCTTGCTGCGCCTCCACCTCCACC
CGTCCTCTGCCTAGCCCCCCCGCGAGCAGCAAGAGCAGGAACCCATGGCG
GGCAAGCAGCGGCAGGAGGAGCGCCAGCGGAGGGAAGAGACGACAGCAGC
TGTCCATCCGCGCGGTGGCCGCACCGTCGTCGGCGGTGGACTACTCGGAC
ACCGCCGCCGGCGCCGGCGACGTCCCCTCGCTGAAAATCAAGCTGCTGAG
CGCGGTCGCCGGGCTGAACCGGGGCCTCGCGGCGAGCCAGGAGGACCTGG
ACCGGGCGGACGCGGCGGCGAGGCAGCTCGAGGCGGCGGCACCGGCCCCC
GTGGACCTCGCCAAGGACCTCGACAAGCTGCAGGGGCGGTGGAGGCTGGT
CTACAGCAGCGCCTTCTCGTCGCGGACGCTCGGCGGCAGCCGCCCCGGCC
CGCCCACCGGCCGCCTCCTCCCCATCACCCTCGGCCAGGTGTTCCAGAGG
ATCGACGTGGTCAGCCAGGACTTCGACAACATCGTGGAGCTCGAGCTCGG
CGCGCCGTGGCCGCTGCCGCCGGTCGAGGCCACGGCCACGCTGGCGCACA
AGTTTGAGATCACCGGAATCGCGAGTATCAAGATCAATTTCGACAAGACG
ACGGTGAAGACCAAAGGGAACCTGTCCCAGCTGCCTCTGCTGGAGGTGCC
CCGCATCCCGGATAGCCTCCGGCCTACGACGTCCAACACCGGGAGCGGCG
AGTTCGACGTGACCTACCTCGACGACGGCACCCGCATCACCCGAGGGGAC
AGGGGGGAGCTCAGGGTGTTCGTCGTCTCATGAGCTGATATTTTTTTTGT
TGATGTTGCTGCTGCTTTCTCTCTCCGTGTACTGCTTCAACCTTTTTGCC
CCTAAACAGAAGTCTTGAACTAGTTCTATGTCTATTTTTGCCGGAGTAGT
ATCGTG

Another example of a receptor in accordance with the present invention is found in wheat and has the amino acid sequence of SEQ ID NO:22 as follows:

MAAPSWSSCCASTSTRPLPSPPASSKGGNPWRASSGRRSASGGKRQQQLS
IRAVAAPSSAVDYSDTGAGAADVPSLKIKLLSAVAGLNRGLAASQEDLDR
ADAAARQLEAAAPAPVDLAKDLDKLQGRWRLVYSSAFSSRTLGGSRPGPP
TGRLLPITLGQVFQRIDVVSQDFDNIVELELGAPWPLPPVEATATLAHKF
EITGIASIKINFDETTVKTNGNLSQLPLLEVPRIPDSLRPPASNTGSGEF
DVTYLDDDTRITRGDRGELRVFVIA

This protein, known as TaHrBP1-2p, is encoded by a cDNA molecule which has a partial sequence corresponding to SEQ ID NO:23 as follows:

ACTAGTGATTCGCGGATCCATATGCTGCGTTTGCTGGCTTTGATGAAACT
CGTGCTCGTCTCTGACCTCTGGCCGGTCGGCACCCAACTGAAAATATCTT
TTCTCGTTGCTCTCCTCGATCGATTGACTGCTTCACCGGACGGTGCCCGT
GGCCATGGCAGCGCCGTCGTGGTCATCTTGCTGCGCCTCCACCTCCACCC
GTCCTCTGCCTAGCCCTCCCGCGAGCAGCAAGGGCGGGAACCCATGGCGG
GCAAGCAGCGGCAGGAGGAGCGCCAGCGGAGGGAAGAGGCAGCAGCAGCT
GTCCATCCGCGCGGTGGCCGCGCCGTCGTCGGCGGTGGACTACTCGGACA
CCGGCGCCGGCGCCGGCGACGTCCCCTCGCTGAAAATCAAGCTGCTGAGC
GCGGTGGCCGGGCTGAACCGGGGCCTCGCGGCGAGCCAGGAGGACCTGGA
CCGGGCGGACGCGGCGGCGAGGCAGCTCGAGGCGGCGGCGCCGGCCCCCG
TGGACCTCGCCAAGGACCTCGACAAGCTGCAGGGGCGGTGGAGGCTGGTC
TACAGCAGCGCCTTCTCGTCGCGGACGCTCGGCGGTAGCCGCCCCGGCCC
GCCCACCGGCCGCCTGCTCCCCATCACCCTCGGCCAGGTGTTCCAGAGGA
TCGACGTGGTGAGCCAGGACTTCGACAACATCGTGGAGCTCGAGCTCGGC
GCGCCGTGGCCGCTGCCGCCGGTGGAGGCCACGGCCACGCTGGCACACAA
GTTTGAGATCACCGGGATCGCGAGTATCAAGATCAATTTCGACGAGACGA
CGGTGAAGACGAATGGGAACCTGTCCCAGCTGCCTCTGCTGGAGGTGCCC
CGCATCCCGGATAGCCTCCGGCCGCCGGCGTCCAACACCGGGAGCGGCGA
GTTCGACGTGACCTACCTCGACGACGACACCCGCATCACCCGAGGGGACA
GGGGGGAGCTCAGGGTGTTCGTCATCGCATGAGCTTGATCTTTGCTTGAG
ATCTCTGTCTCTGTACTGCTTCACTTTTTTTGCCCCGAAACAGAAGTCTT
TGTCTAGTTCTATGTCTTCTTTTGCCGGCGTAGTATTGTGATATAGGCTA
ACGTGCGTTCTTCACCTATGGGATTAACTTTTTCTCTCTAGCAGATTATT
ACGTCCGGTTATTTCGTTTTGGTTTTATTATGTTGGCTTAAGTTTTAATT
ATGTG

Another example of a receptor in accordance with the present invention is found in maize and has the amino acid sequence of SEQ ID NO:24 as follows:

MAATWSSSCCAATASSSALLRHARVKSAPWVAGASRSSYRQRRRRRELSI
RATAAAPPPPVVYADAGADNVASLKIKLLSAVSGLNRGLAASQEDLDRAD
AAARELEAAAGCPVDLSRDLDKLQGRWRLLYSSAFSSRTLGGSRPGPPTG
RLLPITLGQVFQRIDVVSRDFDNIVELELGAPWPLPPLEATATLAHKFEI
IGTSGIKITFEKTTVKTKGNLSQLPPLEVPRIPDNLRPPSNTGSGEFEVT
YLDDDTRVTRGDRGELRVFVIA

This protein, known as ZmHrBP1p, is encoded by a cDNA molecule which has a partial sequence corresponding to SEQ ID NO:25 as follows:

CCACCACAAATATTCTTCCCGCCACGATCCCTCTCATCCGGAAGAAAGGG
GAAAAAAACTCGCCTTTTTCTCTCTGCTGGTTCAAGAACGCCATGGAAGA
TCTCGAGCGCTCGCTGTGATTCCTGCGAGTACCCAAGCCCAACCAAGCCC
TGGCCCGGCAGCCATTCTCTTCGCGCCACATCGCACGACCTCCCCGAAGC
AGACGTGCCCGCTGCCCGTCCGTCCCCCGTGGCCATGGCCGCGACGTGGT
CTTCGTCTTGCTGCGCCGCGACCGCGTCGAGCAGCGCTCTGCTTCGTCAT
GCCCGCGTCAAGAGCGCGCCTTGGGTAGCCGGTGCCAGCCGGAGTAGCTA
CAGGCAGCGGCGGCGGCGGCGGGAGCTGTCCATCCGCGCCACGGCCGCGG
CGCCGCCGCCGCCCGTGGTCTACGCGGACGCCGGCGCCGACAACGTGGCC
TCGCTGAAGATCAAGCTCCTGAGCGCGGTGTCCGGGCTGAACCGTGGCCT
GGCAGCGAGCCAGGAGGACCTGGACCGCGCGGACGCGGCGGCGCGGGAGC
TGGAGGCGGCGGCGGGGTGCCCCGTCGACCTCAGCAGGGACCTCGATAAG
CTGCAGGGCCGGTGGCGGCTGCTGTACAGCAGCGCGTTCTCTTCGCGGAC
GCTCGGCGGCAGCCGCCTTGGCCCGCCCACCGGCCGCCTCCTCCCCATCA
CGCTCGGCCAGGTGTTCCAGCGGATCGACGTGGTGAGCCGCGACTTCGAC
AACATCGTGGAGCTGGAGCTCGGCGCGCCGTGGCCTCTGCCGCCGCTCGA
GGCCACGGCGACGCTGGCGCACAAGTTCGAGATCATCGGGACCTCGGGCA
TCAAGATCACGTTCGAGAAGACGACGGTGAAGACCAAGGGCAACCTGTCG
CAGCTTCCTCCGCTGGAGGTGCCCCGCATCCCGGACAACCTCCGCCCCCC
GTCCAACACCGGGAGCGGCGAGTTCGAGGTGACCTACCTCGACGACGACA
CGCGCGTCACCCGCGGGGACAGGGGGGAGCTCAGGGTGTTTGTCATCGCG
TGACCTGATCGCGCTTCGGCGCCGTTCTGCTGGTCCGTGAGATTGCCATC
CTTCTTCCTCCCTGTTGCTCCAGTAGATTTGTTGGTTTCTTCGTCTGACC
AATGTATACCGTTCTGTTCTTCCGTGAACTGAATCTGCGATTAACTTAGT
AACTATCTTGTGTGGTTT

Another example of a receptor in accordance with the present invention is found in grapefruit and has an amino acid sequence of SEQ ID NO:26 as follows:

MASLTLTPLFHSPTFLSSNTNTHTVTKKLSFPSPTRRRLLVNGKEYRSRR
RSLVLRRSAVDDVPVLDPPPPPPPDSSESDKTELIASLKLKLLSAVSGLN
RGLAANTDDLQKADAAAKELEAVGGPVDLSVGLDRLQGKWRLLYSSAFSS
RTLGGNRPGPPTGRLLPITLGQVFQRIDILSKDFDNIAELELGVPWPLPP
VEVTATLAHKFELIGSSNIKIIFEKTTVKTTGNLSQLPPLELPRFPDALR
RPSDTRSGEFEVTYLDNDTRITRGDRGELRVFVIT

This protein, known as CpHrBP1p, is encoded by a cDNA molecule which has a sequence corresponding to SEQ ID NO:27 a follows:

TTCGATTGCCAGACGCTGCGTTTGCTGGCTTTGATGAAACCTCTTTCATT
CCCTGCTGGCCACAAACACACGCCGACATTGAAACTCCCCCCACCCACAT
CATGGCTTCTCTGACTCTAACCCCTCTTTTTCATTCACCAACATTTCTTT
CCAGCAATACTAACACACACACAGTCACAAAAAAACTGTCTTTTCCATCT
CCAACGCGACGTCGTCTGCTTGTTAATGGTAAGAGTATCGAAGTAGAAGA
AGAAGCCTTGTTTTGAGGAGGTCAGCCGTTGATGACGTTCCTGTTCTTGA
CCCACCACTCCTCCTCCTCCCGATTCTTCAGAAAGCGACAAAACTGAGCT
CATTGCTTCTTTGAAGCTCAAGTTGCTTAGTGCTGTTTCTGGGCTGAACA
GAGGTCTTGCTGCAAACACAGATGATCTGCAGAAGGCAGACGCTGCTGCA
AAAGAGCTTGAGGCTGTTGGAGGACCAGTAGACCTCTCGGTTGGTCTCGA
TAGACTACAAGGGAAATGGAGACTACTGTACAGCAGTGCATTCTCATCTC
GCACTCTAGGTGGAAATCGGCCTGGACCTCCCACTGGAAGGCTACTCCCC
ATAACTCTTGGCCAGGTCTTTCAACGGATTGACATCTTAAGCAAAGATTT
TGATAACATAGCAGAACTTGAATTGGGTGTTCCATGGCCCCTGCCACCAG
TTGAAGTGACTGCCACATTAGCCCATAAATTTGAACTCATAGGATCATCA
AATATTAAAATAATATTTGAGAAGACAACTGTAAAGACAACAGGGAACTT
ATCACAGCTTCCACCCCTTGAGTTACCTCGTTTTCCAGATGCATTAAGGC
GTCCATCTGACACAAGAAGTGGTGAATTTGAGGTGACATACCTCGATAAT
GATACCCGCATTACCAGAGGAGACAGAGGCGAGCTAAGAGTTTTCGTGAT
CACTTAGGTTCCTTACATCCGTACAGTTTCCAGCTTGTATCTACATTATT
TTCTGATGATTATATACACAAAGTGGTAAAAAGAAGCCCCGTGAAAAGCA
GTTCTTCCTGGATCAAGTGAATCATTGCACAATTATATATTTTTCATGCG
C

Another example of a receptor in accordance with the present invention is found in apple and has an amino acid sequence of SEQ ID NO:28 as follows:

MAMASLSSLPHSLHSSPSTSSANYVIPSKPPCPKRLRFGSSNRRHTKSFA
PRAAVDEVSVLEPPPPQPPSSGSKTTPNPELVASLKLNLLSAVSGLNRGL
AASGEDLQKAEAAAKEIEAAGGPVDLSTDLDKLQGRWKLIYSSAFSSRTL
GGSRPGPPTGRLLPITLGQVFQRIDIFSKDFDNIVELELGAPWPLPPVEA
TATLAHKFELIGSSRVKIIFEKTTVKTTGNLSQLPPLELPKLPEGLRPPS
NPGSGEFDVTYLDADIRITRGDRDELRVFVVS

This protein, known as MdHrBP1p is encoded by a cDNA molecule which has a sequence corresponding to SEQ ID NO:29 as follows:

GGCTTTGATGAAATTTCCTTTCTACTTTCTAGCCATGGCCATGGCTTCTT
TGAGCTCTCTCCCTCACTCTCTACATTCCTCGCCTTCTACTTCTTCTGCA
AACTATGTTATTCCAAGCAAACCACCCTGCCCAAAACGCCTCCGTTTTGG
TTCGTCAAATCGCCGTCACACCAAAAGCTTTGCTCCGAGAGCAGCTGTGG
ACGAGGTTTCTGTTCTCGAACCGCCGCCACCACAGCCGCCGTCTTCCGGA
AGCAAAACCACGCCCAACCCTGAACTTGTAGCGTCTTTAAAGCTCAACCT
ATTGAGTGCTGTTTCTGGGCTAAATAGAGGTCTTGCAGCATCGGGAGAGG
ATCTACAAAAGGCAGAAGCTGCTGCCAAGGAGATTGAAGCTGCTGGAGGT
CCAGTGGATCTCTCAACTGATCTTGATAAACTGCAAGGGAGATGGAAATT
GATATATAGCAGTGCATTTTCTTCTCGTACTCTAGGTGGGAGCCGTCCTG
GACCTCCCACCGGAAGGCTACTCCCAATTACCTTAGGCCAGGTATTTCAA
CGGATTGACATCTTCAGCAAAGACTTTGATAACATAGTGGAGCTTGAACT
AGGTGCTCCATGGCCCCTGCCACCCGTTGAAGCAACTGCCACTTTGGCCC
ACAAATTTGAACTCATAGGATCTTCCAGGGTTAAGATCATTTTTGAGAAA
ACTACTGTGAAGACTACTGGAAACTTATCGCAGCTTCCTCCATTAGAGTT
ACCTAAGTTACCGGAAGGACTACGACCTCCGTCTAACCCAGGAAGTGGTG
AATTTGACGTTACCTACCTTGATGCTGATATCCGCATCACAAGAGGAGAT
AGAGACGAGCTAAGGGTTTTTGTTGTTTCATAGTTTCTTGTTAGTTTCTT
TTCCTACTTCCAATGTATCTCCATCTGTTTTGCCTTGCGTCTTCTTGGTG
TCGTTTGATCATATGTTGTTACTTCCAATTGTTGTATGCATGAACCGGTG
GATGGAAGTTCCAGGAAATGTTCAACGAGGAACAACACTGTATACATGTA
AATTTTGTAATCGATAAAGTGAATCGTCTTTGTCACTTGGATTGTATCTG
CATTGCCTTTTCAAGTGATATCTATATGAGTTTTAGGC

Another example of a receptor in accordance with the present invention is found in tobacco and has an amino acid sequence of SEQ ID NO:30 as follows:

MASLLQYSTLPLSNNHCSSSLPSLTCHLSKRSNRNTQKLLEKKKYHIKKS
LICQSGIDELAFIELPGTKEAKAELIGSLKLKLLSAVSGLNRGLAASEED
LKKADAAAKELESCAGAVDLSADLDKLQGRWKLIYSSAFSGRTLGGSRPG
PPTGRLLPITLGQVFQRIDVLSKDFDNIVELELGAPWPLPPAELTATLAH
KFELIGSSTIKITFEKTTVKTTGILSQLPPFEVPRIPDQLRPPSNTGSGE
FEVTYIDSDTRVTRGDRGELRVFVIS

This protein, known as NtHrBP1p, is encoded by a cDNA molecule which has a sequence corresponding to SEQ ID NO:31 as follows:

ATTCACAAACCTTTCCAAATATTGAGCTGAAATTAAAGCTCAACAATGGC
TTCTCTACTTCAGTACTCTACACTTCCTCTTTCTAATAATCATTGTTCAT
CTTCGTTACCATCTTTAACTTGTCATCTCTCAAAAAGAAGCAATAGAAAT
ACTCAAAAATTATTAGAGAAAAAGAAGTATCATATCAAGAAAAGCTTAAT
TTGCCAGTCGGGTATTGATGAACTCGCATTCATTGAGTTACCTGGTACTA
AAGAAGCTAAAGCTGAACTTATTGGGTCTCTCAAACTCAAGTTATTGAGT
GCTGTTTCTGGGCTAAACAGAGGTCTTGCTGCGAGCGAAGAAGACCTAAA
GAAGGCGGATGCTGCTGCCAAGGAGCTAGAATCCTGTGCAGGAGCTGTAG
ATCTCTCAGCTGATCTCGATAAACTTCAAGGGAGGTGGAAATTGATATAC
AGCAGTGCATTCTCAGGTCGCACTCTTGGAGGAAGTCGTCCTGGACCCCC
CACCGGAAGACTTCTTCCCATTACTCTTGGTCAGGTATTTCAAAGAATTG
ATGTGCTAAGCAAGGATTTTGACAACATAGTGGAGCTTGAATTAGGTGCT
CCTTGGCCTTTACCACCTGCTGAGTTGACTGCCACTTTAGCCCACAAATT
TGAACTGATAGGATCATCCACGATTAAGATTACATTCGAGAAAACTACTG
TGAAGACAACCGGAATCTTATCACAGCTCCCACCATTTGAGGTGCCTCGG
ATACCAGATCAACTCAGGCCACCATCTAATACAGGAAGTGGTGAGTTTGA
AGTTACCTATATTGATTCTGATACACGCGTAACAAGGGGAGACAGAGGAG
AGCTTAGAGTTTTCGTTATCTCATAAGATGGAATGCAATAGATATAGTTT
TCCTACAATATTTTGTTGCTACAATTTCATGTACAATATATCAAATGTAT
AGATATGCTCAACATTATTCTGCTGGTCCATATCTAGCAAAGTTGTAATG
TTACTGCAAATTTGAATCTGTATACAGTAAACTCGATTTTGCGA

Another example of a receptor in accordance with the present invention is found in grape and has an amino acid sequence of SEQ ID NO:32 as follows:

MTSLLHPLTSFSLSPSPPPPLSSSSSSTITITCALPSNLRSSDRRRLRTT
SKPYTWTSGLPKRSFVLRSTLDEVSVLDPPPPPEDSTADLLSSLKLKLLS
AVSGLNRGLAAIEDDLQKADAAAKELEAAGGTVDLSIDLDKLQGRWKLIY
SSAFSSRTLGGSRPGPPTGRLLPITLGQVFQRIDIVSKDFDNIVDLQIGV
PWPLPPIELTATLAHKFELIGTSSIKITFEKTTVKTTGNLSQLPPLEVPR
IPDALRPPSNTGSGEFEVTYLDADTRITRGDRGELRVFVIA

This protein, known as VsHrBP1-1p, is encoded by a cDNA molecule which has a sequence corresponding to SEQ ID NO:33 as follows:

ACCGCCAGCCAACTATGACTTCTCTCCTCCATCCTCTCACCTCTTTCTCC
CTTTCTCCATCACCACCACCGCCCCTTTCTTCTTCTTCTTCTTCTACTAT
TACTATCACGTGTGCTCTTCCCAGTAACCTACGTTCTTCAGACCGACGTC
GTCTTAGAACAACATCAAAACCTTATACGTGGACATCGGGCCTGCCCAAG
AGAAGCTTTGTCCTGAGGTCAACCCTTGATGAGGTCTCTGTTCTTGACCC
CCCTCCTCCCCCTGAAGACTCCACGGCCGATCTTCTTTCGTCTCTCAAGC
TGAAACTACTGAGTGCTGTGTCTGGTCTAAATAGAGGACTTGCTGCAATC
GAGGATGATCTTCAGAAGGCAGATGCTGCTGCCAAAGAGCTTGAAGCTGC
TGGAGGAACTGTTGACCTCTCAATTGATCTTGATAAACTTCAGGGAAGAT
GGAAATTGATATATAGCAGTGCGTTCTCATCCCGTACTCTAGGTGGGAGC
CGTCCTGGACCTCCCACTGGAAGGCTACTCCCTATAACTCTGGGCCAGGT
ATTTCAAAGGATTGACATTGTAAGCAAAGATTTTGACAATATAGTAGATC
TCCAGATAGGTGTCCCATGGCCCCTTCCGCCAATTGAACTCACTGCCACA
TTAGCCCACAAGTTTGAACTCATAGGAACTTCCAGCATTAAAATAACATT
CGAGAAAACAACTGTGAAGACAACAGGAAACCTGTCGCAGCTGCCACCAT
TGGAGGTACCTCGGATCCCAGATGCATTGAGGCCACCATCTAATACAGGA
AGTGGCGAATTTGAGGTTACATACCTTGATGCTGATACCCGCATCACCAG
AGGAGACAGGGGTGAGCTTAGAGTTTTTGTCATTGCATAAACTCTAAGCA
CTCGTCACCATGACTCACAATTGAAGAAAATACCATATCCAATCCCCTTT
TCTTCTTGTCATTTTGTAAACAGTCCCCTGTTTCTTACTGTTTGTAGGGA
ACATGTCTTGTTACATATAACTGTAAATTCATTTTTTT

Another example of a receptor in accordance with the present invention is found in grape and has an amino acid sequence of SEQ ID NO:34 as follows:

MTSLLHPLTSFSLSPSPPPPLSFSSSSSTITITCALPSNLRSSDRRRLRT
TSKPYTWTSGLPKRSFVLRSTLDEVSVLDPPPPPEDSTADLLSSLKLKLL
STVSGLNRGLAAIEDDLQKADAAAKELEAAGGTVDLSIDLDKLQGRWKLI
YSSAFSSRTLGGSRPGPPTGRLLPITLGQVFQRIDIVSKDFDNIVDLQIG
APWPLPPIELTATLAHKFELIGTSSIKITFEKTTVKTTGNLSQLPPLEVP
RIPDALRPPSNTGSGEFEVTYLDADTRITRGDRGELRVFVIA

This protein, known as VsHrBP1-2p, is encoded by a cDNA molecule which has a sequence corresponding to SEQ ID NO:35 as follows:

ACCGCCAGCCAACTATGACTTCTCTCCTCCATCCTCTCACCTCTTTCTCC
CTTTCTCCATCACCACCACCGCCCCTTTCTTTTTCTTCTTCTTCTTCTAC
TATTACTATCACGTGTGCTCTTCCCAGTAACCTACGTTCTTCAGACCGAC
GTCGTCTTAGAACAACATCAAAACCTTATACGTGGACATCGGGCCTGCCC
AAGAGAAGCTTTGTCCTGAGGTCAACCCTTGATGAGGTCTCTGTTCTTGA
CCCCCCTCCTCCCCCTGAAGACTCCACGGCCGATCTTCTTTCGTCTCTCA
AACTGAAACTACTGAGTACTGTGTCTGGTCTAAATAGAGGACTTGCTGCA
ATCGAGGATGATCTTCAGAAGGCAGATGCTGCTGCCAAAGAGCTTGAAGC
TGCTGGAGGAACTGTTGACCTCTCAATTGATCTTGATAAACTTCAGGGAA
GATGGAAATTGATATATAGCAGTGCGTTCTCATCCCGTACTCTAGGTGGG
AGCCGTCCTGGACCTCCCACTGGAAGGCTACTCCCTATAACTCTGGGGCA
GGTATTTCAAAGGATTGACATTGTAAGCAAAGATTTTGACAATATAGTAG
ATCTCCAGATAGGTGCCCCATGGCCCCTTCCGCCAATTGAACTCACTGCC
ACATTAGCCCACAAGTTTGAACTCATAGGAACTTCCAGCATTAAAATAAC
ATTCGAGAAAACAACTGTGAAGACAACAGGAAACCTGTCGCAGCTTCCAC
CATTGGAGGTACCTCGGATCCCAGATGCATTGAGGCCACCATCTAATACA
GGAAGTGGCGAATTTGAGGTTACATACCTTGATGCTGATACCCGCATCAC
CAGAGGAGACAGGGGTGAGCTTAGAGTTTTTGTCATTGCATAAACTCTAC
ACTCGTCACCATGACTCACAATTGAAGAAAATACAATATCCAATCCCCTT
TTCTTCTTGTCATTTTGTAAACTGTCCCCTGTTTCTTACTGTTTGTAGGG
AACATGTCTTGTTACATAACTGTAAATTCATTTTTTCTACATTTGATCTT
TACAG

Hypersensitive response elicitors recognized by the receptors of the present invention are able to elicit local necrosis in plant tissue contacted by the elicitor.

Examples of suitable bacterial sources of hypersensitive response elicitor polypeptides or proteins include Erwinia, Pseudomonas, and Xanthamonas species (e.g., the following bacteria: Erwinia amylovora, Erwinia chrysanthemi, Erwinia stewartii, Erwinia carotovora, Pseudomonas syringae, Pseudomonas solancearum, Xanthomonas campestris, and mixtures thereof).

An example of a fungal source of a hypersensitive response elicitor protein or polypeptide is Phytophthora. Suitable species of Phytophthora include Phytophthora parasitica, Phytophthora cryptogea, Phytophthora cinnamomi, Phytophthora capsici, Phytophthora megasperma, and Phytophthora citrophthora.

The hypersensitive response elicitor polypeptide or protein from Erwinia chrysanthemi is disclosed in U.S. Pat. Nos. 5,850,015 and 6,001,959, which are hereby incorporated by reference. This hypersensitive response elicitor polypeptide or protein has a molecular weight of 34 kDa, is heat stable, has a glycine content of greater than 16%, and contains substantially no cysteine.

The hypersensitive response elicitor polypeptide or protein derived from Erwinia amylovora has a molecular weight of about 39 kDa, has a pI of approximately 4.3, and is heat stable at 100° C. for at least 10 minutes. This hypersensitive response elicitor polypeptide or protein has a glycine content of greater than 21% and contains substantially no cysteine. The hypersensitive response elicitor polypeptide or protein derived from Erwinia amylovora is more fully described in U.S. Pat. No. 5,849,868 to Beer and Wei, Z.-M., et al., “Harpin, Elicitor of the Hypersensitive Response Produced by the Plant Pathogen Erwinia amylovora,” Science 257:85-88 (1992), which are hereby incorporated by reference.

The hypersensitive response elicitor polypeptide or protein derived from Pseudomonas syringae has a molecular weight of 34-35 kDa. It is rich in glycine (about 13.5%) and lacks cysteine and tyrosine. Further information about the hypersensitive response elicitor derived from Pseudomonas syringae and its encoding DNA molecule is found in U.S. Pat. Nos. 5,708,139 and 5,858,786 and He et al., “Pseudomonas syringae pv. syringae Harpinₚₛₛ: A Protein that is Secreted via the Hrp Pathway and Elicits the Hypersensitive Response in Plants,” Cell 73:1255-66 (1993), which are hereby incorporated by reference.

The hypersensitive response elicitor polypeptide or protein derived from Pseudomonas solanacearum is set forth in Arlat, M., F. Van Gijsegem, J. C. Huet, J. C. Pemollet, and C. A. Boucher, “PopA1, a Protein which Induces a Hypersensitive-like Response in Specific Petunia Genotypes, is Secreted via the Hrp Pathway of Pseudomonas solanacearum,” EMBO J. 13:543-533 (1994), which is hereby incorporated by reference. This protein has 344 amino acids, a molecular weight of 33.2 kDa, and a pI of 4.16, is heat stable and glycine rich (20.6%).

The hypersensitive response elicitor polypeptide or protein from Xanthomonas campestris pv. glycines has a partial amino acid sequence corresponding to SEQ ID NO:36 as follows:

```
Thr Leu Ile Glu Leu Met Ile Val Val Ala Ile Ile Ala Ile Leu Ala
    1 5 10 15
Ala Ile Ala Leu Pro Ala Tyr Gln Asp Tyr
                          20 25
```

This sequence is an amino terminal sequence having only 26 residues from the hypersensitive response elicitor polypeptide or protein of Xanthomonas campestris pv. glycines. It matches with fimbrial subunit proteins determined in other Xanthomonas campestris pathovars.

The hypersensitive response elicitor polypeptide or protein from Xanthomonas campestris pv. pelargonii is heat stable, protease sensitive, and has a molecular weight of 12 kDa. It has the amino acid sequence of SEQ ID NO:37 as follows:

```
Met Asp Ser Ile Gly Asn Asn Phe Ser Asn Ile Gly Asn Leu Gln Thr
    1 5 10 15
Met Gly Ile Gly Pro Gln Gln His Glu Asp Ser Ser Gln Gln Ser Pro
                          20 25 30
Ser Ala Gly Ser Glu Gln Gln Leu Asp Gln Leu Leu Ala Met Phe Ile
                  35 40 45
Met Met Met Leu Gln Gln Ser Gln Gly Ser Asp Ala Asn Gln Glu Cys
          50 55 60
Gly Asn Glu Gln Pro Gln Asn Gly Gln Gln Glu Gly Leu Ser Pro Leu
  65 70 75 80
Thr Gln Met Leu Met Gln Ile Val Met Gln Leu Met Gln Asn Gln Gly
                                  85 90 95
Gly Ala Gly Met Gly Gly Gly Gly Ser Val Asn Ser Ser Leu Gly Gly
                        100 105 110
Asn Ala
```

This amino acid sequence is encoded by the nucleotide sequence of SEQ ID NO:38 as follows:

| | |
|---|---|
| atggactcta tcggaaacaa cttttcgaat atcggcaacc tgcagacgat gggcatcggg | 60 |
| cctcagcaac acgaggactc cagccagcag tcgccttcgg ctggctccga gcagcagctg | 120 |
| gatcagttgc tcgccatgtt catcatgatg atgctgcaac agagccaggg cagcgatgca | 180 |
| aatcaggagt gtggcaacga acaaccgcag aacggtcaac aggaaggcct gagtccgttg | 240 |
| acgcagatgc tgatgcagat cgtgatgcag ctgatgcaga accagggcgg cgccggcatg | 300 |
| ggcggtggcg gttcggtcaa cagcagcctg ggcggcaacg cc | 342 |

Isolation of Erwinia carotovora hypersensitive response elictor protein or polypeptide is described in Cui et al., “The RsmA Mutants of Erwinia carotovora subsp. carotovora Strain Ecc71 Overexpress hrp N_{ECC} and Elicit a Hypersensitive Reaction-like Response in Tobacco Leaves,” MPMI 9(7):565-73 (1996), which is hereby incorporated by reference. This protein has 356 amino acids, a molecular weight of 35.6 kDa, and a pI of 5.82 and is heat stable and glycine rich (21.3%).

The hypersensitive response elicitor protein or polypeptide of Erwinia stewartii is set forth in Ahmad et al., “Harpin is Not Necessary for the Pathogenicity of Erwinia stewartii on Maize,” 8th Int'l. Cong. Molec. Plant-Microbe Interact., Jul. 14-19, 1996 and Ahmad, et al., “Harpin is Not Necessary for the Pathogenicity of Erwinia stewartii on Maize,” Ann. Mtg. Am. Phytopath. Soc., Jul. 27-31, 1996, which are hereby incorporated by reference.

Hypersensitive response elicitor proteins or polypeptides from Phytophthora parasitica, Phytophthora cryptogea, Phytophthora cinnamoni, Phytophthora capsici, Phytophthora megasperma, and Phytophora citrophthora are described in Kaman, et al., “Extracellular Protein Elicitors from Phytophthora: Most Specificity and Induction of Resistance to Bacterial and Fungal Phytopathogens,” Molec. Plant-Microbe Interact. 6(1):15-25 (1993), Ricci et al., “Structure and Activity of Proteins from Pathogenic Fungi Phytophthora Eliciting Necrosis and Acquired Resistance in Tobacco,” Eur. J. Biochem. 183:555-63 (1989), Ricci et al., “Differential Production of Parasiticein, and Elicitor of Necrosis and Resistance in Tobacco, by Isolates of Phytophthora parasitica,” Plant Path. 41:298-307 (1992), Baillreul et al, “A New Elicitor of the Hypersensitive Response in Tobacco: A Fungal Glycoprotein Elicits Cell Death, Expression of Defence Genes, Production of Salicylic Acid, and Induction of Systemic Acquired Resistance,” Plant J. 8(4):551-60 (1995), and Bonnet et al., “Acquired Resistance Triggered by Elicitors in Tobacco and Other Plants,” Eur. J. Plant Path. 102:181-92 (1996), which are hereby incorporated by reference. These hypersensitive response elicitors from Phytophthora are called eliciting. All known elicitins have 98 amino acids and show >66% sequence identity. They can be classified into two groups, the basic elicitins and the acidic eliciting, based on the physicochemical properties. This classification also corresponds to differences in the eliciting ability to elicit HR-like symptoms. Basic elicitins are 100 times more effective than the acidic ones in causing leaf necrosis on tobacco plants.

The hypersensitive response elicitor from Gram positive bacteria like Clavibacter michiganesis is described in WO 99/11133, which is hereby incorporated by reference.

The above elicitors are exemplary. Other elicitors can be identified by growing fungi or bacteria that elicit a hypersensitive response using conditions under which genes encoding an elicitor are expressed. Cell-free preparations from culture supernatants can be tested for elicitor activity (i.e. local necrosis) by using them to infiltrate appropriate plant tissues.

Turning again to the receptor of the present invention for such hypersensitive response elicitors, fragments of the above receptor protein are encompassed by the method of the present invention. In addition, fragments of full length receptor proteins from other plants can also be utilized.

Suitable fragments can be produced by several means. In the first, subclones of the gene encoding a known receptor protein are produced by conventional molecular genetic manipulation by subcloning gene fragments. The subclones then are expressed in vitro or in vivo in bacterial cells to yield a smaller protein or peptide that can be tested for receptor activity according to the procedure described above.

As an alternative, fragments of a receptor protein can be produced by digestion of a full-length receptor protein with proteolytic enzymes like chymotrypsin or Staphylococcus proteinase A, or trypsin. Different proteolytic enzymes are likely to cleave receptor proteins at different sites based on the amino acid sequence of the receptor protein. Some of the fragments that result from proteolysis may be active receptors.

In another approach, based on knowledge of the primary structure of the receptor protein, fragments of the receptor protein gene may be synthesized by using the PCR technique together with specific sets of primers chosen to represent particular portions of the protein. These then would be cloned into an appropriate vector for expression of a truncated peptide or protein.

Chemical synthesis can also be used to make suitable fragments. Such a synthesis is carried out using known amino acid sequences for the receptor being produced. Alternatively, subjecting a full length receptor to high temperatures and pressures will produce fragments. These fragments can then be separated by conventional procedures (e.g., chromatography, SDS-PAGE).

Variants may be made, for example, by altering the gene by the addition of bases encoding amino acids that have minimal influence on the properties, secondary structure, and hydropathic nature of the encoded polypeptide. For example, a polypeptide may be produced that has a signal (or leader) sequence at the N-terminal end of the protein product that co-translationally or post-translationally directs transfer of the protein. The polypeptide, via gene alteration, may also be conjugated to a short 6-10 residue tag or other sequence for ease of synthesis, purification, or identification of the polypeptide

Suitable DNA molecules are those that hybridize to a DNA molecule comprising a nucleotide sequence of 50 continuous bases of SEQ ID NO:2, SEQ ID NO:7, SEQ ID NO:9, SEQ ID NO:13, SEQ ID NO:19, SEQ ID NO:27, SEQ ID NO:29, SEQ ID NO:31, SEQ ID NO:33, SEQ ID NO:35, or SEQ ID NO:39 under stringent conditions characterized by hybridization in buffer comprising 0.9M sodium citrate (“SSC”) buffer at a temperature of 37° C. and remaining bound when subject to washing with the SSC buffer at 37° C.; and preferably in a hybridization buffer comprising 20% formamide in 0.9M saline/0.09M SSC buffer at a temperature of 42° C. and remaining bound when subject to washing at 42° C. with 0.2×SSC buffer at 42° C.

The receptor of the present invention is preferably produced in purified form (preferably at least about 60%, more preferably 80%, pure) by conventional techniques. Typically, the receptor of the present invention is produced but not secreted into the growth medium of recombinant host cells. Alternatively, the receptor protein of the present invention is secreted into growth medium. In the case of unsecreted protein, to isolate the receptor protein, the host cell (e.g., E. coli) carrying a recombinant plasmid is propagated, lysed by sonication, or chemical treatment, and the homogenate is centrifuged to remove bacterial debris. The cell lysate can be further purified by conventionally utilized chromatography procedures (e.g., gel filtration in an appropriately sized dextran or polyacrylamide column to separate the receptor protein). If necessary, the protein fraction may be farther purified by ion exchange or HPLC.

The DNA molecule encoding the receptor protein can be incorporated in cells using conventional recombinant DNA technology. Generally, this involves inserting the DNA molecule into an expression system to which the DNA molecule is heterologous (i.e. not normally present). The heterologous DNA molecule is inserted into the expression system or vector in sense orientation and correct reading frame. The vector contains the necessary elements for the transcription and translation of the inserted protein-coding sequences.

U.S. Pat. No. 4,237,224 to Cohen and Boyer, which is hereby incorporated by reference, describes the production of expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture.

Recombinant genes may also be introduced into viruses, such as vaccina virus. Recombinant viruses can be generated in virus infected cells transformed with plasmids.

Suitable vectors include, but are not limited to, the following viral vectors such as lambda vector system gt11, gt WES.tB, Charon 4, and plasmid vectors such as pBR322, pBR325, pACYC177, pACYC1084, pUC8, pUC9, pUC18, pUC19, pLG339, pR290, pKC37, pKC101, SV 40, pBluescript II SK ± or KS ± (see “Stratagene Cloning Systems” Catalog (1993) from Stratagene, La Jolla, Calif., which is hereby incorporated by reference), pQE, pIH821, pGEX, pET series (see F. W. Studier et. al., “Use of T7 RNA Polymerase to Direct Expression of Cloned Genes,” Gene Expression Technology vol. 185 (1990), which is hereby incorporated by reference), and any derivatives thereof. Recombinant molecules can be introduced into cells via transformation, transduction, conjugation, mobilization, or electroporation. The DNA sequences are cloned into the vector using standard cloning procedures in the art, as described by Sambrook et al., Molecular Cloning: A Laboratory Manual, Cold Spring Harbor Laboratory Press, Cold Spring Harbor, N.Y., third edition (2001), which is hereby incorporated by reference.

A variety of host-vector systems may be utilized to express the protein-encoding sequence(s). Primarily, the vector system must be compatible with the host cell used. Host-vector systems include but are not limited to the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and plant cells infected by bacteria. The expression elements of these vectors vary in their strength and specificities. Depending upon the host-vector system utilized, any one of a number of suitable transcription and translation elements can be used.

Different genetic signals and processing events control many levels of gene expression (e.g., DNA transcription and messenger RNA (mRNA) translation).

Transcription of DNA is dependent upon the presence of a promotor which is a DNA sequence that directs the binding of RNA polymerase and thereby promotes mRNA synthesis. The DNA sequences of eucaryotic promoters differ from those of procaryotic promotors. Furthermore, eucaryotic promoters and accompanying genetic signals may not be recognized in or may not function in a procaryotic system, and, further, procaryotic promotors are not recognized and do not function in eucaryotic cells.

Similarly, translation of mRNA in procaryotes depends upon the presence of the proper procaryotic signals which differ from those of eucaryotes. Efficient translation of mRNA in procaryotes requires a ribosome binding site called the Shine-Dalgarno (“SD”) sequence on the mRNA. This sequence is a short nucleotide sequence of mRNA that is located before the start codon, usually AUG, which encodes the amino-terminal methionine of the protein. The SD sequences are complementary to the 3′-end of the 16S rRNA (ribosomal RNA) and probably promote binding of mRNA to ribosomes by duplexing with the rRNA to allow correct positioning of the ribosome. For a review on maximizing gene expression, see Roberts and Lauer, Methods in Enzymology 68:473 (1979), which is hereby incorporated by reference.

Promotors vary in their “strength” (i.e. their ability to promote transcription). For the purposes of expressing a cloned gene, it is desirable to use strong promoters in order to obtain a high level of transcription and, hence, expression of the gene. Depending upon the host cell system utilized, any one of a number of suitable promotors may be used. For instance, when cloning in E. coli, its bacteriophages, or plasmids, promotors such as the T7 phage promoter, lac promotor, trp promotor, recA promotor, ribosomal RNA promotor, the P_{R} and P_{L} promotors of coliphage lambda and others, including but not limited, to lacUV5, ompF, b/a, lpp, and the like, may be used to direct high levels of transcription of adjacent DNA segments. Additionally, a hybrid trp-lacUV5 (tac) promotor or other E. coli promotors produced by recombinant DNA or other synthetic DNA techniques may be used to provide for transcription of the inserted gene.

Bacterial host cell strains and expression vectors may be chosen which inhibit the action of the promotor unless specifically induced. In certain operations, the addition of specific inducers is necessary for efficient transcription of the inserted DNA. For example, the lac operon is induced by the addition of lactose or IPTG (isopropylthio-beta-D-galactoside). A variety of other operons, such as trp, pro, etc., are under different controls.

Specific initiation signals are also required for efficient gene transcription and translation in procaryotic cells. These transcription and translation initiation signals may vary in “strength” as measured by the quantity of gene specific messenger RNA and protein synthesized, respectively. The DNA expression vector, which contains a promotor, may also contain any combination of various “strong” transcription and/or translation initiation signals. For instance, efficient translation in E. coli requires an SD sequence about 7-9 bases 5′ to the initiation codon (“ATG”) to provide a ribosome binding site. Thus, any SD-ATG combination that can be utilized by host cell ribosomes may be employed. Such combinations include but are not limited to the SD-ATG combination from the cro gene or the N gene of coliphage lambda, or from the E. coli tryptophan E, D, C, B or A genes. Additionally, any SD-ATG combination produced by recombinant DNA or other techniques involving incorporation of synthetic nucleotides may be used.

Once the isolated DNA molecule encoding the receptor protein has been cloned into an expression system, it is ready to be incorporated into a host cell. Such incorporation can be carried out by the various forms of transformation noted above, depending upon the vector/host cell system. Suitable host cells include, but are not limited to, bacteria, virus, yeast, mammalian cells, insect, plant, and the like.

One aspect of the present invention involves enhancing a plant's receptivity to treatment with a hypersensitive response elicitor by providing a transgenic plant or transgenic plant seed, transformed with a nucleic acid molecule encoding a receptor protein for a hypersensitive response elicitor. It has been found that hypersensitive response elicitors are useful in imparting disease resistance to plants, enhancing plant growth, effecting insect control and/or imparting stress resistance in a variety of plants. In view of the receptor of the present invention's interaction with such elicitors, it is expected that these beneficial effects would be enhanced by carrying out such elicitor treatments with plants transformed with the receptor encoding gene of the present invention.

Transgenic plants containing a gene encoding a receptor in accordance with the present invention can be prepared according to techniques well known in the art.

A vector containing the receptor encoding gene described above can be microinjected directly into plant cells by use of micropipettes to transfer mechanically the recombinant DNA. Crossway, Mol. Gen. Genetics 202:179-85 (1985), which is hereby incorporated by reference. The genetic material may also be transferred into the plant cell using polyethylene glycol. Krens, et al., Nature 296:72-74 (1982), which is hereby incorporated by reference.

Another approach to transforming plant cells with a gene is particle bombardment (also known as biolistic transformation) of the host cell. This can be accomplished in one of several ways. The first involves propelling inert or biologically active particles at cells. This technique is disclosed in U.S. Pat. Nos. 4,945,050, 5,036,006, and 5,100,792, all to Sanford et al., which are hereby incorporated by reference. Generally, this procedure involves propelling inert or biologically active particles at the cells under conditions effective to penetrate the outer surface of the cell and to be incorporated within the interior thereof. When inert particles are utilized, the vector can be introduced into the cell by coating the particles with the vector containing the heterologous DNA. Alternatively, the target cell can be surrounded by the vector so that the vector is carried into the cell by the wake of the particle. Biologically active particles (e.g., dried bacterial cells containing the vector and heterologous DNA) can also be propelled into plant cells.

Yet another method of introduction is fusion of protoplasts with other entities, either minicells, cells, lysosomes, or other fusible lipid-surfaced bodies. Fraley, et al., Proc. Natl. Acad. Sci. USA 79:1859-63 (1982), which is hereby incorporated by reference.

The DNA molecule may also be introduced into the plant cells by electroporation. Fromm et al., Proc. Natl. Acad. Sci. USA 82:5824 (1985), which is hereby incorporated by reference. In this technique, plant protoplasts are electroporated in the presence of plasmids containing the expression cassette. Electrical impulses of high field strength reversibly permeabilize biomembranes allowing the introduction of the plasmids. Electroporated plant protoplasts reform the cell wall, divide, and regenerate.

Another method of introducing the DNA molecule into plant cells is to infect a plant cell with Agrobacterium tumefaciens or A. rhizogenes previously transformed with the gene. Under appropriate conditions known in the art, the transformed plant cells are grown to form shoots or roots, and develop further into plants. Generally, this procedure involves inoculating the plant tissue with a suspension of bacteria and incubating the tissue for 48 to 72 hours on regeneration medium without antibiotics at 25-28° C.

Agrobacterium is a representative genus of the gram-negative family Rhizobiaceae. Its species are responsible for crown gall (A. tumefaciens) and hairy root disease (A. rhizogenes). The plant cells in crown gall tumors and hairy roots are induced to produce amino acid derivatives known as opines, which are catabolized only by the bacteria. The bacterial genes responsible for expression of opines are a convenient source of control elements for chimeric expression cassettes. In addition, assaying for the presence of opines can be used to identify transformed tissue.

Heterologous genetic sequences can be introduced into appropriate plant cells, by means of the Ti plasmid of A. tumefaciens or the Ri plasmid of A. rhizogenes. The Ti or Ri plasmid is transmitted to plant cells on infection by Agrobacterium and is stably integrated into the plant genome. J. Schell, Science 237:1176-83 (1987), which is hereby incorporated by reference.

After transformation, the transformed plant cells must be regenerated.

Plant regeneration from cultured protoplasts is described in Evans et al., Handbook of Plant Cell Cultures, Vol. 1: (MacMillan Publishing Co., New York, 1983); and Vasil I. R. (ed.), Cell Culture and Somatic Cell Genetics of Plants, Acad. Press, Orlando, Vol. I, 1984, and Vol. III (1986), which are hereby incorporated by reference.

It is known that practically all plants can be regenerated from cultured cells or tissues, including but not limited to, all major species of sugarcane, sugar beets, cotton, fruit trees, and legumes.

Means for regeneration vary from species to species of plants, but generally a suspension of transformed protoplasts or a petri plate containing transformed explants is first provided. Callus tissue is formed and shoots may be induced from callus and subsequently rooted. Alternatively, embryo formation can be induced in the callus tissue. These embryos germinate as natural embryos to form plants. The culture media will generally contain various amino acids and hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

After the expression cassette is stably incorporated in transgenic plants, it can be transferred to other plants by sexual crossing. Any of a number of standard breeding techniques can be used, depending upon the species to be crossed.

Once transgenic plants of this type are produced, the plants themselves can be cultivated in accordance with conventional procedures. Alternatively, transgenic seeds or propagules (e.g., cuttings) are recovered from the transgenic plants. The seeds can then be planted in the soil and cultivated using conventional procedures to produce transgenic plants. The transgenic plants are propagated from the planted transgenic seeds.

These elicitor treatment methods can involve applying the hypersensitive response elicitor polypeptide or protein in a non-infectious form to all or part of a plant or a plant seed transformed with a receptor gene in accordance with the present invention under conditions effective for the elicitor to impart disease resistance, enhance growth, control insects, and/or to impart stress resistance. Alternatively, the hypersensitive response elicitor protein or polypeptide can be applied to plants such that seeds recovered from such plants themselves are able to impart disease resistance in plants, to enhance plant growth, to effect insect control, and/or to impart resistance to stress.

As an alternative to applying a hypersensitive response elicitor polypeptide or protein to plants or plant seeds in order to impart disease resistance in plants, to effect plant growth, to control insects, and/or to impart stress resistance in the plants or plants grown from the seeds, transgenic plants or plant seeds can be utilized. When utilizing transgenic plants, this involves providing a transgenic plant transformed with both a DNA molecule encoding a receptor in accordance with the present invention and with a DNA molecule encoding a hypersensitive response elicitor polypeptide or protein. The plant is grown under conditions effective to permit the DNA molecules to impart disease resistance to plants, to enhance plant growth, to control insects, and/or to impart resistance to stress. Alternatively, a transgenic plant seed transformed with a DNA molecule encoding a hypersensitive response elicitor polypeptide or protein and a DNA molecule encoding a receptor can be provided and planted in soil. A plant is then propagated from the planted seed under conditions effective to permit the DNA molecules to impart disease resistance to plants, to enhance plant growth, to control insects, and/or to impart resistance to stress.

The embodiment where the hypersensitive response elicitor polypeptide or protein is applied to the plant or plant seed can be carried out in a number of ways, including: 1) application of an isolated elicitor or 2) application of bacteria which do not cause disease and are transformed with a gene encoding the elicitor. In the latter embodiment, the elicitor can be applied to plants or plant seeds by applying bacteria containing the DNA molecule encoding the hypersensitive response elicitor polypeptide or protein. Such bacteria must be capable of secreting or exporting the elicitor so that the elicitor can contact plant or plant seeds cells. In these embodiments, the elicitor is produced by the bacteria in planta or on seeds or just prior to introduction of the bacteria to the plants or plant seeds.

The hypersensitive response elicitor treatment can be utilized to treat a wide variety of plants or their seeds to impart disease resistance, enhance growth, control insects, and/or impart stress resistance. Suitable plants include dicots and monocots. More particularly, useful crop plants can include: alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane. Examples of suitable ornamental plants are: Arabidopsis thaliana, Saintpaulia, petunia, pelargonium, poinsettia, chrysanthemum, carnation, and zinnia.

With regard to the use of hypersensitive response elicitors in imparting disease resistance, absolute immunity against infection may not be conferred, but the severity of the disease is reduced and symptom development is delayed. Lesion number, lesion size, and extent of sporulation of fungal pathogens are all decreased. This method of imparting disease resistance has the potential for treating previously untreatable diseases, treating diseases systemically which might not be treated separately due to cost, and avoiding the use of infectious agents or environmentally harmful materials.

The method of imparting pathogen resistance to plants is useful in imparting resistance to a wide variety of pathogens including viruses, bacteria, and fungi. Resistance, inter alia, to the following viruses can be achieved by the method of the present invention: Tobacco mosaic virus and Tomato mosaic virus. Resistance, inter alia, to the following bacteria can also be imparted to plants Pseudomonas solancearum; Pseudomonas syringae pv. tabaci; and Xanthamonas campestris pv. pelargonii. Plants can be made resistant, inter alia, to the following fungi: Fusarium oxysporum and Phytophthora infestans.

With regard to the use of the hypersensitive response elicitor protein or polypeptide to enhance plant growth, various forms of plant growth enhancement or promotion can be achieved. This can occur as early as when plant growth begins from seeds or later in the life of a plant. For example, plant growth according to the present invention encompasses greater yield, increased quantity of seeds produced, increased percentage of seeds germinated, increased plant size, greater biomass, more and bigger fruit, earlier fruit coloration, and earlier fruit and plant maturation. As a result, there is significant economic benefit to growers. For example, early germination and early maturation permit crops to be grown in areas where short growing seasons would otherwise preclude their growth in that locale. Increased percentage of seed germination results in improved crop stands and more efficient seed use. Greater yield, increased size, and enhanced biomass production allow greater revenue generation from a given plot of land.

The use of hypersensitive response elicitors for insect control encompasses preventing insects from contacting plants to which the hypersensitive response elicitor has been applied, preventing direct insect damage to plants by feeding injury, causing insects to depart from such plants, killing insects proximate to such plants, interfering with insect larval feeding on such plants, preventing insects from colonizing host plants, preventing colonizing insects from releasing phytotoxins, etc. The present invention also prevents subsequent disease damage to plants resulting from insect infection.

Elicitor treatment is effective against a wide variety of insects. European corn borer is a major pest of corn (dent and sweet corn) but also feeds on over 200 plant species including green, wax, and lima beans and edible soybeans, peppers, potato, and tomato plus many weed species. Additional insect larval feeding pests which damage a wide variety of vegetable crops include the following: beet armyworm, cabbage looper, corn ear worm, fall armyworm, diamondback moth, cabbage root maggot, onion maggot, seed corn maggot, pickleworm (melonworm), pepper maggot, tomato pinworm, and maggots. Collectively, this group of insect pests represents the most economically important group of pests for vegetable production worldwide.

Hypersensitive response elicitor treatment is also useful in imparting resistance to plants against environmental stress. Stress encompasses any environmental factor having an adverse effect on plant physiology and development. Examples of such environmental stress include climate-related stress (e.g., drought, water, frost, cold temperature, high temperature, excessive light, and insufficient light), air pollution stress (e.g., carbon dioxide, carbon monoxide, sulfur dioxide, NOₓ, hydrocarbons, ozone, ultraviolet radiation, acidic rain), chemical (e.g., insecticides, fungicides, herbicides, heavy metals), and nutritional stress (e.g., fertilizer, micronutrients, macronutrients).

The application of the hypersensitive response elicitor polypeptide or protein can be carried out through a variety of procedures when all or part of the plant is treated, including leaves, stems, roots, etc. This may (but need not) involve infiltration of the hypersensitive response elicitor polypeptide or protein into the plant. Suitable application methods include high or low pressure spraying, injection, and leaf abrasion proximate to when elicitor application takes place. When treating plant seeds or propagules (e.g., cuttings), the hypersensitive response elicitor protein or polypeptide can be applied by low or high pressure spraying, coating, immersion, or injection. Other suitable application procedures can be envisioned by those skilled in the art provided they are able to effect contact of the elicitor with cells of the plant or plant seed. Once treated with a hypersensitive response elicitor, the seeds can be planted in natural or artificial soil and cultivated using conventional procedures to produce plants. After plants have been propagated from seeds treated with an elicitor, the plants may be treated with one or more applications of the hypersensitive response elicitor protein or polypeptide to impart disease resistance to plants, to enhance plant growth, to control insects on the plants, and/or to impart stress resistance.

The hypersensitive response elicitor polypeptide or protein can be applied to plants or plant seeds alone or in a mixture with other materials. Alternatively, the elicitor can be applied separately to plants with other materials being applied at different times.

A composition suitable for treating plants or plant seeds contains a hypersensitive response elicitor polypeptide or protein in a carrier. Suitable carriers include water, aqueous solutions, slurries, or dry powders.

Although not required, this composition may contain additional additives including fertilizer, insecticide, fungicide, nematacide, and mixtures thereof. Suitable fertilizers include (NH₄)₂NO₃. An example of a suitable insecticide is Malathion. Useful fungicides include Captan.

Other suitable additives include buffering agents, wetting agents, coating agents, and abrading agents. In addition, the hypersensitive response elicitor can be applied to plant seeds with other conventional seed formulation and treatment materials, including clays and polysaccharides.

In the alternative technique involving the use of transgenic plants and transgenic seeds encoding a hypersensitive response elicitor encoding gene, a hypersensitive response elicitor need not be applied topically to the plants or seeds. Instead, transgenic plants transformed with a DNA molecule encoding such an elicitor are produced according to procedures well known in the art as described above.

In another embodiment, the present invention relates to a DNA construct which is an antisense nucleic acid molecule to a nucleic acid molecule encoding a receptor in plants for plant pathogen hypersensitive response elicitors. An example of such a construct would be an antisense DNA molecule of the DNA molecule having the nucleotide sequence of SEQ ID NO:2, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35 (or a portion thereof). Alternatively, the DNA construct can have a DNA molecule having the nucleotide sequence of SEQ ID NO:2, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35 (or a portion thereof) and its complementary strand and is used to generate a single transcript with an inverted repeat (i.e. a double-stranded) RNA. This transcript as well as the above-discussed antisense nucleic acid molecule can be used to induce silencing of a nucleic acid molecule encoding a receptor for a hypersensitive response elicitor.

Sensing the hypersensitive response elicitor by the receptor is the very first step of the signal transduction pathway in plants which eventually leads to disease resistance, growth enhancement, insect control, and stress resistance. Silencing the receptor provides a powerful tool to find and study the downstream components of this pathway. Additionally, the receptor could be a negative regulator of such plant signal transduction pathway. Silencing of the receptor will impart to plants the ability to resist disease and stress, control insects, and enhance growth without hypersensitive response elicitor treatment.

### EXAMPLES

### Example 1

### Materials and Methods

The laboratory techniques used in the following example are routine. All DNA manipulations described here followed conventional protocols (Sambrook et al., “Molecular Cloning: A Laboratory Manual,” 2^{nd} ed., Cold Spring Harbor Laboratory (1989); Ausubel, et al., “Current Protocols in Molecular Biology,” John Wiley (1987), which are hereby incorporated by reference). The plasmids and microorganisms described herein, used for making the present invention, were obtained from commercial sources, or from the authors of previous publications. Sequences were analyzed with Clone Manager 5 (Scientific & Educational Software, Durham, N.C.).

Yeast strain L40 was grown in YPD or in different minimal synthetic dropout selection media at 30° C. E. coli strains DH5α and HB101 were grown in LB at 37° C.

The yeast Two-Hybrid system is based on the fact that many eukaryotic transcription factors are composed of a physically separable, functionally independent DNA-binding domain (DNA-BD) and an activation domain (AD). Both the DNA-BD and the AD are required to activate a gene. When physically separated by recombinant DNA technology and expressed in the same host cell, the DNA-BD and the AD do not interact directly with each other and, thus, cannot activate the responsive gene (Ma, et al., “Converting a Eukaryotic Transcriptional Inhibitor into an Activator,” Cell 55:443 (1988) and Brent, et al., “A Eukaryotic Transcriptional Activator Bearing the DNA Specificity of a Prokaryotic Repressor,” Cell 43:729 (1985), which are hereby incorporated by reference). But if the DNA-BD and the AD are brought into close physical proximity in the promoter region, the transcriptional activation function will be restored. Therefore, the yeast Saccharomyces cerevisiae and the Two-Hybrid system have become essential genetic tools for studying the macromolecular interactions.

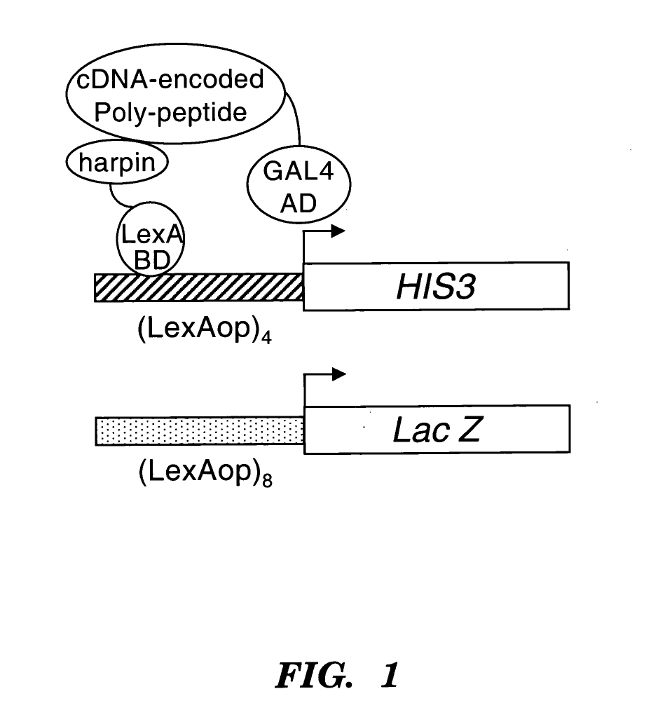

In the Two-Hybrid system utilized here, the DNA-BD, encoded in the bait vector pVJL11 (Jullien-Flores, V., “Bridging Ral GTPase to Rho Pathways. RLIP76, a Ral Effector with CDC42/Rac GTPase-activating Protein Activity,” J. Biol. Chem. 27:22473 (1995), which is hereby incorporated by reference), is the prokaryotic LexA protein, and the activation domain, encoded in the prey vector pGAD 10 or pGAD GH (Clontech; Hannon, G J., “Isolation of the Rb-related p130 Through its Interaction with CDK2 and Cyclins,” Genes Dev. 7:2378 (1993), which is hereby incorporated by reference) is derived from the yeast GAL4 protein. pVJ11 also has a TRP1 marker, and the pGAD has a LEU2 marker. An interaction between the bait protein (fused to the DNA-BD) and a library-encoded protein (fused to the AD) creates a novel transcriptional activator with binding affinity for LexA operators. The yeast host L40 {MATa his3D200 trp1-901 leu2-3, 112 ade2 LYS2::(lexAop)₄-HIS3 URA3::(lexAop)₈-lacZ} harbors two reporter genes, lacZ and HIS3, which contain upstream LexA binding site. The HIS3 nutritional reporter provides a sensitive growth selection that can identify a single positive transformant out of several million candidate clones. The expression of the reporter genes indicates interaction between a candidate protein and the bait protein. See FIG. 1.

Erwinia amylovora harpin was used as the bait protein to screen the Arabidopsis thaliana MATCHMAKER cDNA library cloned in the pGAD 10 vector (Clontech Laboratories, Inc., Palo Alto, Calif.). One cDNA library encoded protein was identified as a strong harpin interacting protein and, thus, a putative harpin receptor. The present invention reports the nucleic acid sequence and the deduced amino acid sequence of this cDNA.

### Example 2

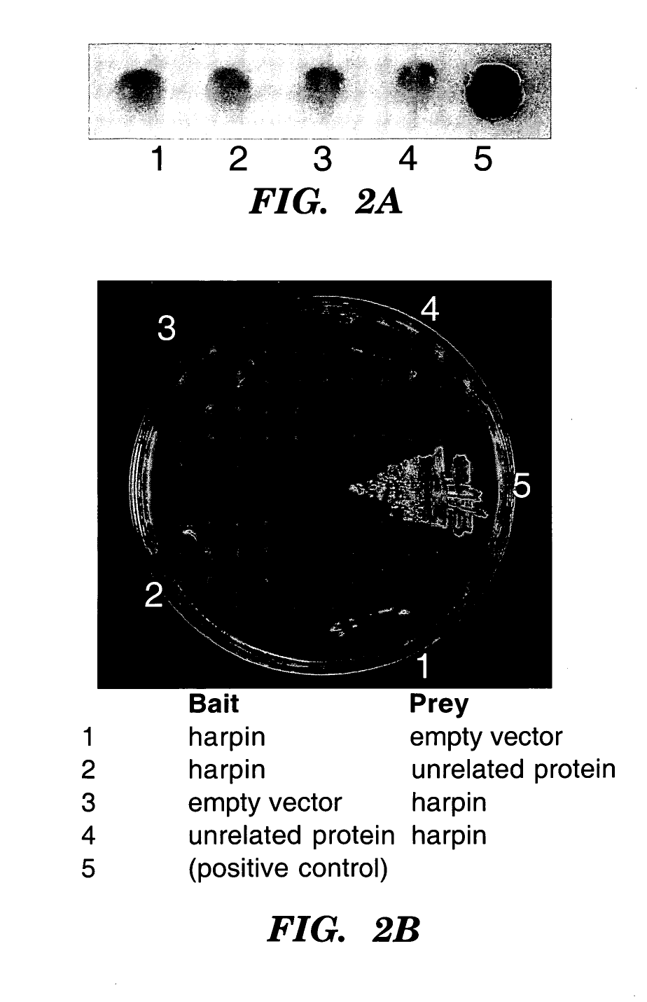

HrpN of Erwinia amylovora was subcloned into the yeast Two-Hybrid bait vector pVJL11. PCR was carried out using the 1.3 kb harpin fragment (Wei et al., “Harpin, Elicitor of the Hypersensitive Response Produced by the Plant Pathogen Erwinia amylovora,” Science 257:85 (1992), which is hereby incorporated by reference) as a template to amplify the harpin encoding region. A BamHI site was added to the 5′ end of the coding sequence, and a SalI site to the 3′ end. A BamHI and SalI digested PCR fragment was ligated with the bait vector pVJL11 digested with the same restriction enzymes. pVJL11 has a TRP1 marker for selection in yeast and an Ampicillin resistance marker for selection in E. coli. The plasmid DNA was amplified in E. coli strain DH5α. When tested in the Two-Hybrid system with empty prey vector pGAD GH and several unrelated proteins, HrpN did not show auto-activation or nonspecific interaction with unrelated proteins, as shown in FIG. 2.

### Example 3

HrpN-pVJL11 was transformed into yeast strain L40 by a lithium acetate (LiAc)-mediated method (Ito et al., “Transformation of Intact Yeast Cells Treated with Alkali Cations,” J. Bacteriol. 153:163 (1983) and Vojtek et al., “Mammalian Ras Interacts Directly with the Serine/Threonine Kinase Raf.,” Cell 74:205 (1993), which are hereby incorporated by reference). The Arabidopsis thaliana MATCHMAKER cDNA library (Clontech Laboratories, Inc., Palo Alto, Calif.) was screened for harpin interacting proteins. Approximately 6.8 million primary library transformants were plated onto plates lacking histidine, leucine, and tryptophan. A total of 148 colonies grew on the histidine dropout plates, 55 of which stained positive when tested for expression of P-galactosidase. After three rounds of selection on synthetic minimal (SD) media plates lacking leucine, tryptophan, and histidine, and confirming by the expression of the second reporter gene lacZ using a β-galactosidase assay, 47 colonies seemed to be strong interacting candidates.

### Example 4

Plasmid DNA was extracted from the 47 independent yeast colonies and shuttled into E. coli strain HB101, which carries the leuB mutation. Therefore, the prey plasmid (cDNA-pGAD 10) was selected for on minimal nutrient plates since pGAD 10 bears the LEU2 marker.

The 47 independently rescued prey plasmids purified from E. coli were retested in the yeast two-hybrid system with harpin as bait. They were also tested against unrelated proteins. 25 turned out to be interacting candidates, 20 of which were strong specific interacting candidates. Sequencing analysis showed that the 20 independent cDNA clones were actually from the same gene with different integrity at their 5′ end. The sequence reactions were performed using the PE Prism BigDye™ dye terminator reaction kit. The sequencing gel was run in Thatagen (Bothell, Wash.).

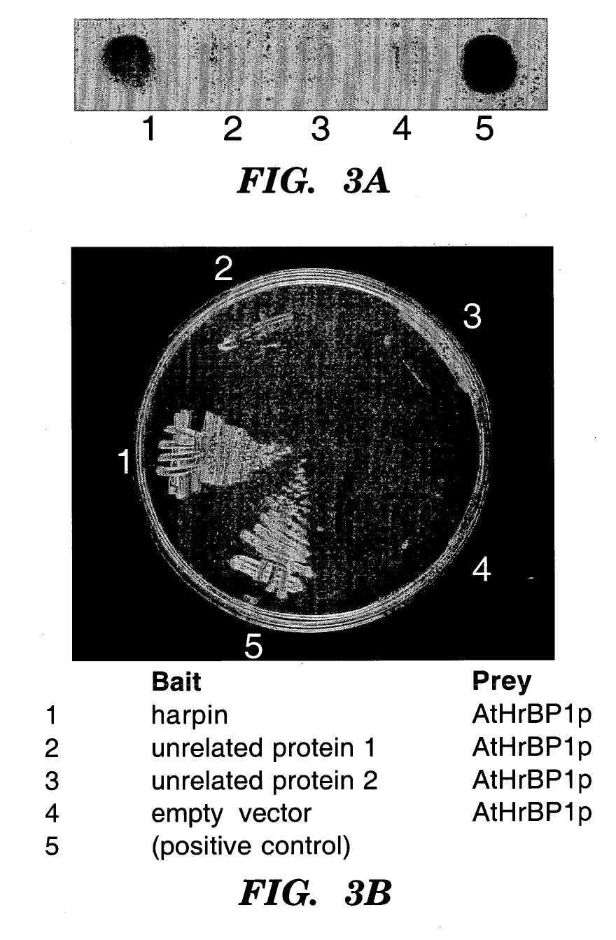

One of the eight plasmids, which had the longest cDNA insert of 1 kb, was used for further analysis. When co-transformed into yeast strain L40, it was shown to be negative with empty bait and unrelated proteins in the Two-Hybrid system, indicating the specificity of the interaction between harpin and this receptor candidate. See FIG. 3.

### Example 5

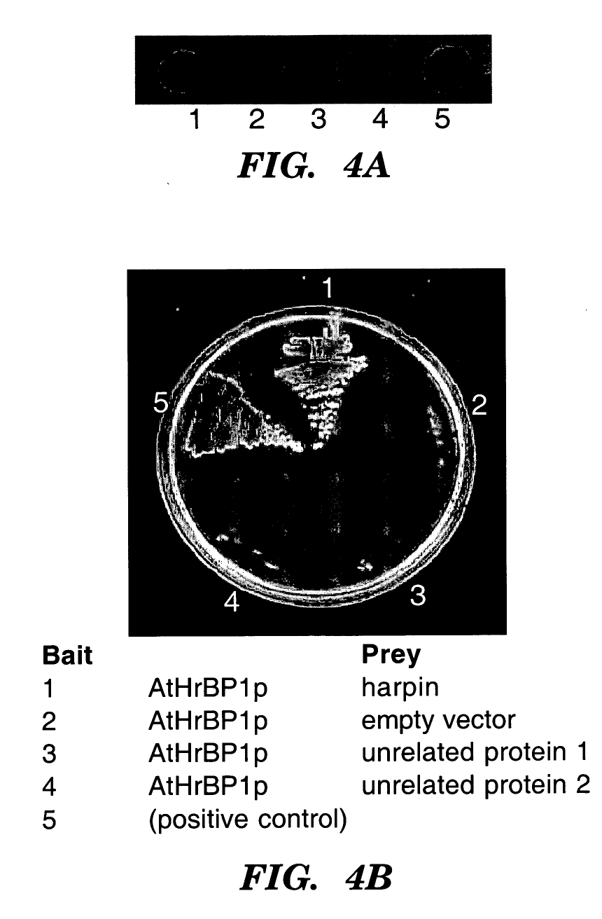

The longest cDNA insert, designated AtHrBP1 (Arabidopsis thaliana harpin-binding-protein 1), was subcloned into the BamHI and SalI sites of the bait vector pVJL11. This construct did not show auto-activation of the reporter genes, nor interaction with unrelated proteins in the yeast Two-Hybrid system. However, the expression of the reporter genes was activated when L40 was co-transformed with AtHrBP1-pVJL11 and hrpN-pGAD GH, indicating the specific interaction between AtHrBP1p (“p” distinguishing the protein encoded by AtHrBP1) and harpin. See FIG. 4.

### Example 6

Total RNA was extracted from two-week-old Arabidopsis thaliana using QIAGEN RNeasy plant mini kit (Qiagen, Inc., Valencia, Calif.). Poly A⁺ RNA was further purified from the total RNA with a QIAGEN Oligotex column (Qiagen, Inc., Valencia, Calif.). A Northern blot was carried out using the translated region of AtHrBP1 as a probe. One single species with an apparent molecular weight of about 1.1 kb was detected from both total RNA and Poly A⁺ RNA. Therefore, the longest cDNA of AtHrBP1 from the yeast two-hybrid screen seems to be the full-length cDNA. The integrity of the 5′ of cDNA was further confirmed by a primer extension assay.

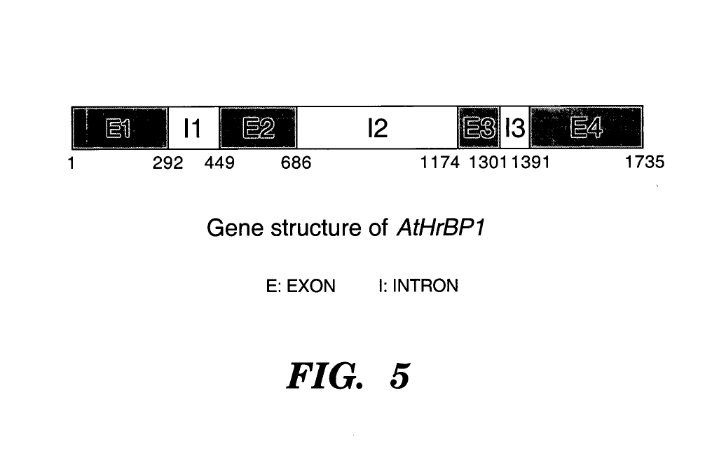

As described, the yeast Two-Hybrid system was used to screen for harpin interacting proteins. hrpN of Erwinia amylovora was subcloned into the yeast Two-Hybrid bait vector pVJL11, which has a TRP1 marker. The lexA-harpin fusion protein is expressed from this construct in yeast. The Arabidopsis thaliana MATCHMAKER cDNA library (Clontech Laboratories, Inc., Palo Alto, Calif.) was screened for hypersensitive response elicitor interacting proteins. 6.8 million independent colonies were screened, and AtHrBP1 was identified as a strong specific harpin interacting candidate. AtHrBP1 was mapped to Arabidopsis thaliana genomic DNA, chromosome 3, P1 clone MLM24 (Nakamura, “Structural Analysis of Arabidopsis thaliana chromosome 3,” Direct submission to the DDBJ/EMBL/GenBank databases (1998), which is hereby incorporated by reference). Four exons and three introns were discovered (See FIG. 5). Exon 4 includes a 130 bp non-translated 3′ region. The in-frame open reading frame from the first methionine encodes a polypeptide of 284 amino acids, AtHrBP1p. The predicted molecular weight of AtHrBP1p is 30454.3 and the predicted pI is 5.72. There is no apparent hydrophobic trans-membrane domain in this polypeptide. The AtHrBP1-AD fusion prey was negative with empty bait and unrelated proteins in the yeast 2-H system, indicating the specificity of the interaction between harpin and this receptor candidate. When tested in the opposite orientation, i.e. AtHrBP1p fused with the DNA-BD and harpin with the AD, they still specifically interacted with each other.

### Example 7

The AtHrBP1 cDNA was subcloned into the NdeI and SalI sites of the vector pET-28a (Novagen, Madison, Wis.). AtHrBP1p was expressed from this vector in E. coli as a His-tagged protein and purified with Ni-NTA resion (QIAGEN Inc., Valencia, Calif.) according to the manual provided by the manufacturer. This recombinant protein increased harpin's ability to induce HR in tobacco plants. Recombinant AtHrBP1p with the His-tag removed was used to generate anti-AtHrBP1p antibody to facilitate biochemical and functional studies of AtHrBP1p. Preliminary localization studies using anti-AtHrBP1p antibody in a Western blot showed that AtHrBP1p exists everywhere in Arabidopsis, including its leaves, stems, roots, flowers and seeds and that it is most likely cell wall bound.

### Example 8

Ten μg of total RNA from 14 different plant species was separated on a 1% agarose gel, and then transferred to Amersham Hybond NX membrane (Amersham Pharmacia Biotech, Piscataway, N.J.). The RNA probe, which was complementary to bases 651-855 of AtHrBP1 coding region, was generated using Ambion Strip-EZ RNA kit (Ambion Inc., Houston, Tex.). Membrane hybridization was done with Ambion ULTRAhyb (Ambion Inc., Houston, Tex.), procedure according to manufacturer recommendation.

The sequence of the AtHrBP1 fragment used to generate the Northern probe (SEQ ID NO:39) is as follows:

| | |
|---|---|
| gatcaagata acatttgaga aaacaactgt gaagacatcg ggaaacttgt cgcagattcc | 60 |
| tccgtttgat atcccgaggc ttcccgacag tttcagacca tcgtcaaacc ctggaactgg | 120 |
| ggatttcgaa gttacctatg ttgatgatac catgcgcata actcgcgggg acagaggtga | 180 |
| acttagggta ttcgtcattg cttaa | 205 |

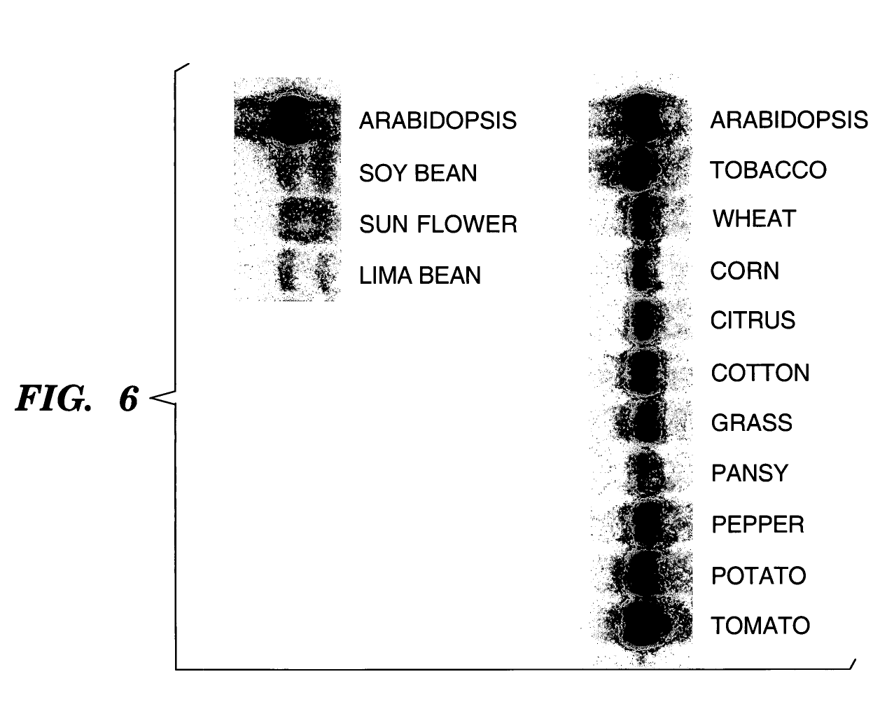

This Northern blot picked up a band with similar size as AtHrBP1 in all the plant species tested, including tobacco, wheat, corn, citrus, cotton, grass, pansy, pepper, potato, tomato, soybean, sun flower, and lima bean. This indicated that HrBP 1-like genes exist universally. See FIG. 6.

### Example 9

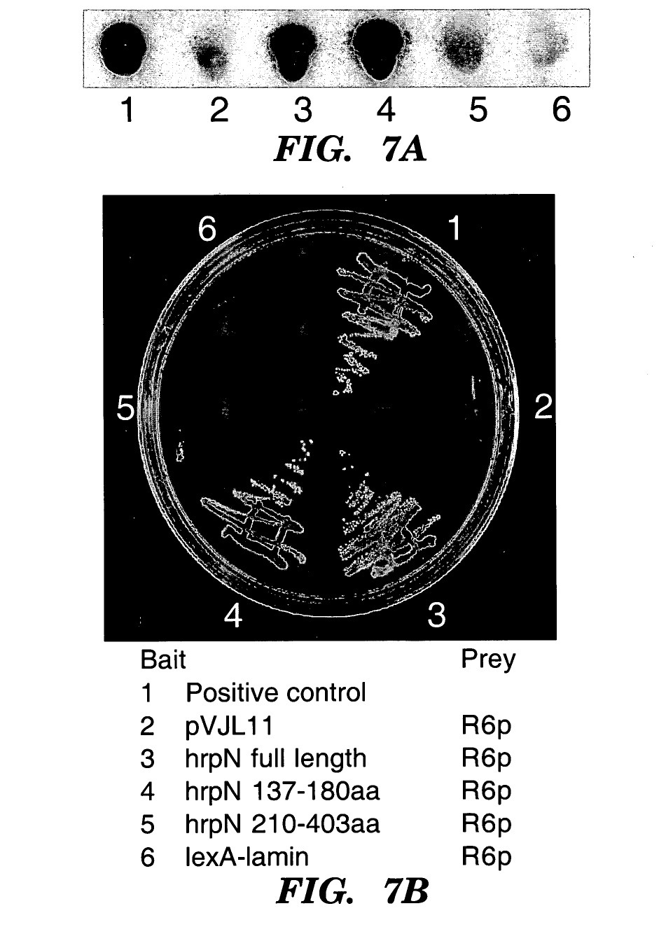

An HrBP1 homologue from rice, R6, was cloned by the yeast two-hybrid screening method, using harpin as bait. It not only interacted with full length harpin but also interacted with a harpin fragment that contains the second HR domain (see FIG. 7). However, it was not a full-length cDNA; there was 5′ end sequence information missing. The R6 partial sequence from rice encoded a peptide of 203 amino acids (R6p). The predicted amino acid sequences for R6p and for AtHrBP1p were compared. Their similarity extended from amino acid 84 through amino acid 284 of AtHrBP1p. The proteins were 74.4% identical and 87.2% similar at the predicted amino acid level, and the two genes were 65% identical at the DNA level.

### Example 10

To obtain a full length rice-HrBP1 homologue, cDNA was prepared from total rice RNA using the R6-specific antisense primer R6NL2 (based on the partial sequence obtained from the yeast two-hybrid screening) (see Table 1) and the 5′ RACE System kit purchased from GIBCO-BRL Life Technologies. The cDNA was then dC-tailed and amplified by the polymerase chain reaction (PCR). The PCR reaction utilized the Advantage-GC2 polymerase mix (Clontech), R6NL1B (see Table 1) as the 3′ primer, and either a polyG-containing primer (Abridged Anchor Primer, Gibco-BRL) or R6NL6 (see Table 1) as the 5′ primer. PCR with the generic primer was performed first. Based on the sequencing results from clones obtained, R6NL6 was designed and used for a second cloning strategy, which started from a fresh batch of RNA (same tissue) and yielded a new batch of clones. The PCR products were gel-purified, cloned into pT-Adv (Clontech), and screened by restriction enzyme digestions prior to sequencing on both strands. Sequencing primers used were: the T7 promoter primer, the M13 reverse primer, R6NL1B, or R6NL4 (see Table 1).

3′ RACE was conducted using a kit and reagents from Ambion (First Choice RLM RACE Kit), which included a polyT primer. The 3′ portion of the R6 gene was amplified by PCR using the R6-specific primer R6NL11 (designed from the partial sequence obtained from the yeast Two-hybrid screening) (see Table 1), a 3′ RACE primer supplied with the kit called 3′ RACE OUTER, and Advantage-GC2 polymerase mix (Clontech). A second round of PCR was done with the R6-specific primer R6NL10 (see Table 1), a 3′ RACE primer supplied with the kit called 3′ RACE INNER, and Advantage-GC2 polymerase mix. The PCR products were cloned into vector pBluescript SK− and sequenced using the T7 promoter primer and the T3 promoter primer.

TABLE 1
Gene-specific primers used in 5′ and 3′ RACE.

| | | | |
|---|---|---|---|
| 5′ RACE outer primer | 5′ RACE inner primer | 3′ RACE outer primer | 3′ RACE inner primer |

| | | | | | | | |
|---|---|---|---|---|---|---|---|
| barley | PB7 | | PB9 | | PB8 | | n/a |
| | ACGAGAAG | (SEQ ID NO:40) | AGCTTGAT | (SEQ ID NO:41) | ACCTCAAC | (SEQ ID NO:42) | |
| | GCGTTGCT | | TTTGAGCG | | CTCCACCC | | |
| | GTAGACCA | | AGGGGATG | | ATTCTC | | |

| | | | | | | |
|---|---|---|---|---|---|---|
| maize | PB16 | | PB3 | | n/a | n/a |
| | CTTCTCGA | (SEQ ID NO:43) | ATGTTGTC | (SEQ ID NO:44) | | |
| | ACGTGATC | | GAAGTCGC | | | |
| | TTGATGC | | GGCTCACC | | | |
| | | | A | | | |

| | | | | | | | | |
|---|---|---|---|---|---|---|---|---|
| potato | PB13 | | PB14 | | PB15 | | PB17 | |
| | TAGCTCCT | (SEQ ID NO:45) | GTGACTTC | (SEQ ID NO:46) | GCTCAACC | (SEQ ID NO:47) | CACTTTTA | (SEQ ID NO:48) |
| | TGGCAGCC | | ATCAATAC | | ATGGCTTC | | TTGAGCCA | |
| | TCAT | | CCGACTG | | TCTACTTC | | CCTGGTAG | |

| | | | | | | | |
|---|---|---|---|---|---|---|---|
| tomato | PB10 | | PB11 | | PB15 | | n/a |
| | CTGACCAA | (SEQ ID NO:49) | CAAGAGTA | (SEQ ID NO:50) | GCTCAACC | (SEQ ID NO:47) | |
| | GAGTGATG | | CGAGATGA | | ATGGCTTC | | |
| | GGAAGAAG | | GAATGCAC | | TCTACTTC | | |

| | | | | | | | | |
|---|---|---|---|---|---|---|---|---|
| wheat | PB19 | | PB20 | | PB21 | | PB22 | |
| | ACGAGAAG | (SEQ ID NO:51) | GCGCTCAG | (SEQ ID NO:52) | TTGCTCTC | (SEQ ID NO:53) | ATCGCCGT | (SEQ ID NO:54) |
| | GCGCTGCT | | CAGCTTGA | | CTCGATCG | | CGTGGTCA | |
| | GTAGAC | | TTTTC | | ATTGAC | | TCTTGC | |

OsHrBP1 3′ and 5′ RACE Primers

| | | |
|---|---|---|
| R6NL2: | CCGATGATCTCAAACTTGTGA | (SEQ ID NO:55) |
| R6NL1B: | GTCCTTGCTGACAACATCGATCCTCTG | (SEQ ID NO:56) |
| R6NL6: | TCGCCATTGATTTTCTCTGTCTGCTC | (SEQ ID NO:57) |
| R6NL4: | GAAGCTTGACTTTGAGCGCAGCCAC | (SEQ ID NO:58) |
| R6NL10: | GACGCCGTGGCTGCGCTCAAAGTCAAG | (SEQ ID NO:59) |
| R6NL11: | GTGGACTACGCGGCGGGCACCGGCG | (SEQ ID NO:60) |

DNA sequences from several clones were aligned using the Clone Manager 5/SE Central suite of programs. Clones fell into 1 of 2 groups that differed in sequence at discrete locations 5′, 3′, and within the R6 sequence. Clones resembling the original R6 sequence obtained from yeast Two-hybrid screening were designated OsHrBP1-1 and the other clones were called OsHrBP1-2. All clones belonged to either the OsHrBP1-1 group or the OsHrBP1-2 group.

### Example 11

The GenBank dBEST and non-redundant databases were searched for HrBP1 gene family members using the AtHrBP1p amino acid sequence and the search program TBLASTN with default parameters (Altschul et al., “Gapped BLAST and PS1-BLAST: A New Generation of Protein Database Search Programs,” Nucl. Acids Res. 25:3389-402 (1997), which is hereby incorporated by reference). Partial HrBP1 cDNA sequences were identified from the following crop plants: barley, maize, potato, soybean, tomato, and wheat.

Appropriate primers were then designed for the above crops, with the exception of soybean, to perform rapid amplification of cDNA ends (RACE) using the FirstChoice RLM-RACE kit (Ambion) according to the manufacturer's instructions. This strategy employs, in the first rounds of amplification, an initial gene-specific primer (outer primer) in combination with an adapter-specific primer, followed by a second round of amplification using another adapter-specific primer and another gene-specific primer (inner primer), which hybridizes downstream of the outer primer region and does not overlap with it. For 3′ RACE a second round of amplification with an inner primer is sometimes not necessary. Table 1 shows sequences of gene-specific primers used in 5′ and 3′ RACE reactions with cDNA samples from the above crop plants. Primers were synthesized by Integrated DNA Technologies, Inc (Coralville, Iowa).

In the case of wheat and grape, the primers listed in Table 1 yielded two different, but highly conserved HrBP1 sequences. Confirmation that the resulting 5′ and 3′ RACE products belonged to the same cDNA was performed by either confirming the identity of overlapping sequences in 5′ and 3′ products, or by isolating full-length cDNAs using 3′ RACE gene-specific primers designed to hybridize in the 5′ untranslated region (UTR).

TABLE 2
Degenerate primers used in 5′ and 3′ RACE.

| | |
|---|---|
| Primer sequence | corresponding to amino acid sequence |

| | | | | |
|---|---|---|---|---|
| 5′ RACE outer primer PB27 | TCRAAYTTRTGNGCNARNGTNGC | (SEQ ID NO:61) | ATLAHKFE | (SEQ ID NO:62) |
| 5′ RACE inner primer PB1 | ATICKYTGRAAIACYTG | (SEQ ID NO:63) | QVFQRI | (SEQ ID NO:64) |
| 3′ RACE outer primer PB24 | GTNWSNGGNYTNAAYMGNGGNYT | (SEQ ID NO:65) | VSGLNRGL | (SEQ ID NO:66) |
| 3′ RACE inner primer PB26 | GGNCARGTNTTYCARMGNATHGA | (SEQ ID NO:67) | GQVFQRID | (SEQ ID NO:68) |

H = A/C/T
I = inosine
K = G/T
M = A/C
N = A/C/G/T
R = A/G
S = C/G
W = A/T
Y = C/T

With respect to the soybean GmHrBP1 sequence, after a partial sequence had been identified from dBEST and non-redundant databases searches, clones were purchased from InCyte Genomics and sequenced. A full length GmHrBP1 sequence was obtained using standard, vector specific sequencing primers.

Comparison of the deduced amino acid sequences of HrBP1 cDNAs thus far obtained, lead to the identification of regions of conserved motifs (further described in Example 12). From these regions, degenerate primers were designed in order to amplify HrBP1-like cDNAs from plants for which no HrBPJ sequences were available. Table 2 shows sequences of successfully used degenerate primers. Degenerate primers were used to amplify 5′ and 3′ RACE products from plant cDNA preparations. Subsequently, specific primers designed to hybridize in the 5′ and 3′ UTR regions were employed to amplify cDNA fragments with a full-length open reading frame. In this manner, HrBP1 sequence information was obtained from species of grapefruit, cotton, apple, tobacco, and grape.

TABLE 3

| | | | amino | | | |
|---|---|---|---|---|---|---|
| | | nucleotides in | acids | | Molecular | Original Genbank |
| Gene name | Plant | longest cDNA | encoded | pI | mass (kDa) | accession number |
| CpHrBP1 | grapefruit | 1103 | 285 | 9.61 | 31.3 | none |
| GhHrBP1 | cotton | 1064 | 277 | 9.37 | 30.0 | none |
| GmHrBP1 | soybean | 1075 | 265 | 7.88 | 28.4 | BG043054 |
| HvHrBP1 | barley | 1129 | 277 | 9.35 | 29.3 | BE216663 |
| LeHrBP1 | tomato | 1026 | 276 | 6.25 | 30.1 | AI779661 |
| MdHrBP1 | apple | 1138 | 282 | 8.96 | 30.2 | none |
| NtHrBP1 | tobacco | 1044 | 276 | 8.80 | 30.0 | none |
| OsHrBP1-1 | rice | 1123 | 270 | 8.92 | 28.4 | none |
| OsHrBP1-2 | rice | 1112 | 269 | 8.56 | 28.2 | none |
| StHrBP1 | potato | 1078 | 275 | 8.31 | 30.1 | BE923126 |
| TaHrBP1-1 | wheat | 1057 | 277 | 9.64 | 29.4 | BG907618 |
| TaHrBP1-2 | wheat | 1205 | 275 | 7.75 | 30.0 | BG908482 |
| VsHrBP1-1 | grape | 1038 | 291 | 7.82 | 31.4 | none |
| VsHrBP1-2 | grape | 1055 | 292 | 7.82 | 31.5 | none |
| ZmHrBP1 | maize | 1218 | 272 | 9.57 | 29.3 | BG319894 |

### Example 12

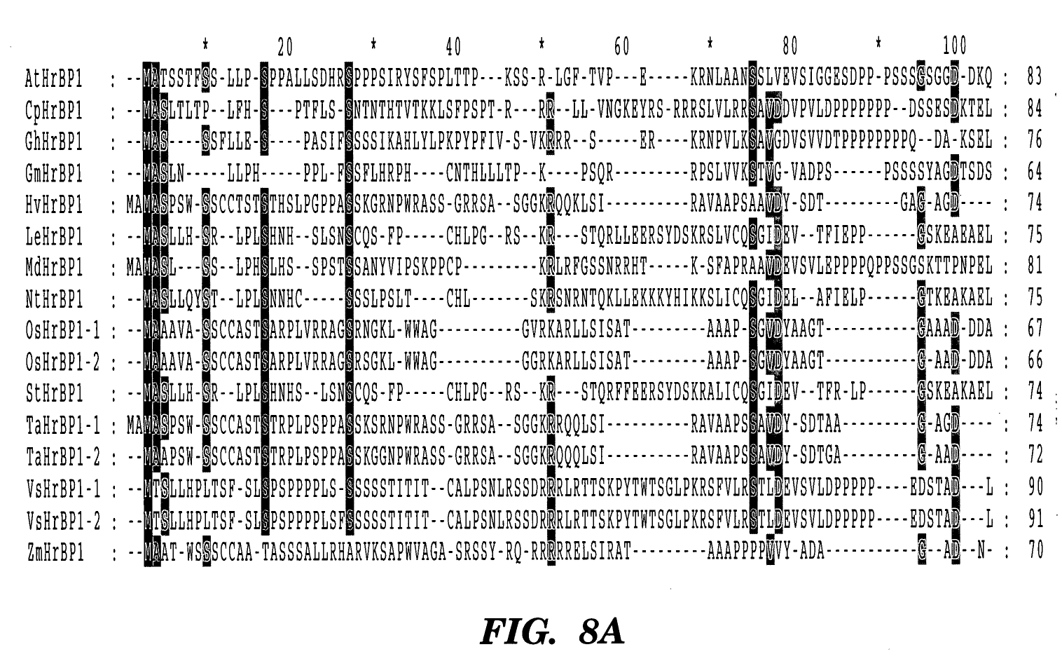

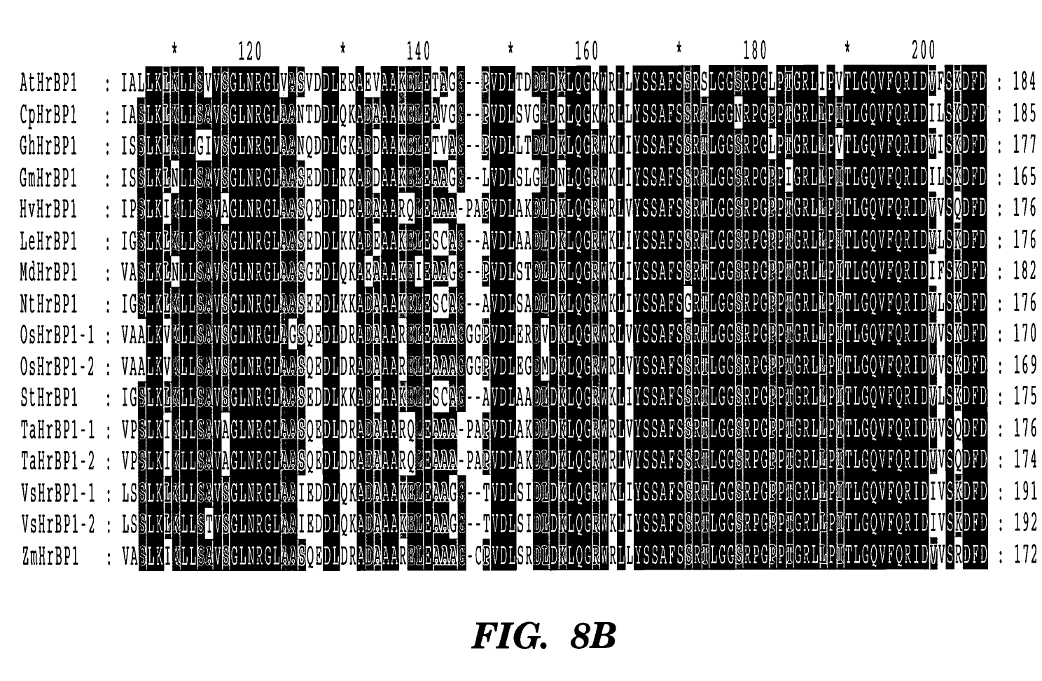

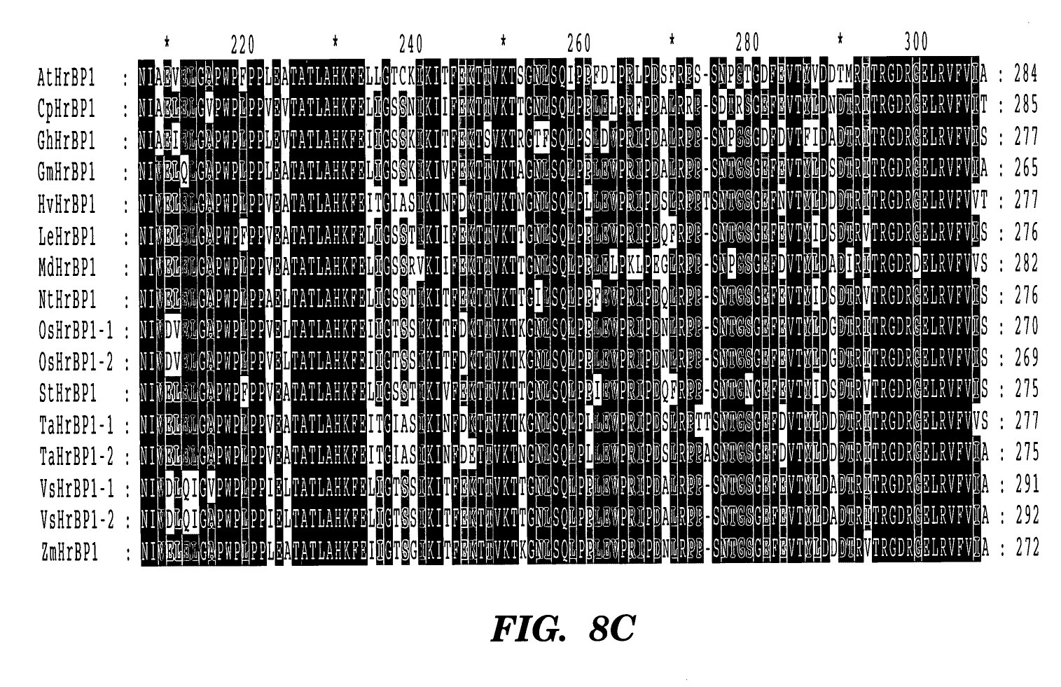

The HrBP 1 amino acid and nucleotide sequences were analyzed and compared using several different techniques. The cDNA open reading frame or amino acid sequences were compared using the program Align Plus 4. DNA comparisons used a standard linear scoring matrix; amino acid comparisons used the BLOSUM 62 scoring matrix (See Tables 4). FIGS. 8A-C show a comprehensive comparison of the HrBP1p amino acid sequences constructed with the use of the GeneDoc program (Nicholas, K. B., Nicholas H. B. Jr., and Deerfield, D. W. II. 1997 GeneDoc: Analysis and Visualization of Genetic Variation, EMBNEW.NEWS 4:14, which is hereby incorporated by reference).

TABLE 4
Percent identity of predicted open reading frame and amino acid sequences of HrBP1 cDNAs. Figures in white boxes
represent DNA sequence identity; figures in shaded boxes represent amino acid sequence identity.

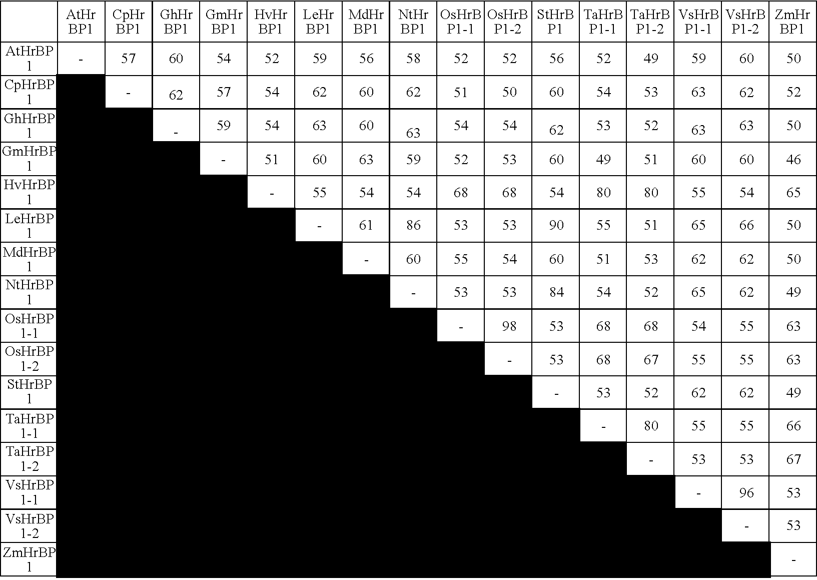

### Example 13

Based on the HrBP1p amino acid comparisons described in Example 13, regions of highly conserved amino acid sequences were identified. Identification of these regions further enabled identification of specific motifs throughout the conserved region of HrBP1p. As a result of this analysis, several blocks of 5 or more identical amino acids were found as shown in Table 5.

TABLE 5

| | |
|---|---|
| Location in AtHrBP1p | |
| (SEQ ID NO:1) | Motif |

| | | |
|---|---|---|
| 97-102 | GLNRGL | (SEQ ID NO:69) |
| 143-148 | YSSAFS | (SEQ ID NO:70) |
| 168-177 | TLGQVFQRID | (SEQ ID NO:71) |
| 182-186 | DFDNI | (SEQ ID NO:72) |
| 203-211 | TATLAHKFE | (SEQ ID NO:73) |
| 271-275 | TRGDR | (SEQ ID NO:74) |
| 277-282 | ELRVFV | (SEQ ID NO:75) |

In addition, several blocks of 5 or more conserved amino acids were found as shown in Table 6.

TABLE 6

| | |
|---|---|
| Location | |
| in AtHrBP1p | Motif |

| | | |
|---|---|---|
| 97-102 | GLNRGL | (SEQ ID NO:69) |
| 115-120 | AA42LE | (SEQ ID NO:76) |
| 135-148 | LQG4W4L6YSSAFS | (SEQ ID NO:77) |
| 150-154 | R3LGG | (SEQ ID NO:78) |
| 162-178 | GRL6P6TLGQVFQRID6 | (SEQ ID NO:79) |
| 182-186 | DFDNI | (SEQ ID NO:72) |
| 203-212 | TATLAHKFE6 | (SEQ ID NO:80) |
| 225-229 | T3VKT | (SEQ ID NO:81) |
| 261-265 | VT56D | (SEQ ID NO:82) |
| 269-275 | R6TRGDR | (SEQ ID NO:83) |
| 277-283 | ELRVFV6 | (SEQ ID NO:84) |

2 = E Q
3 = S T
4 = K R
5 = F W Y
6 = I L M V

The information presented in Table 5 can be combined to define the receptor of the present invention as having an amino acid sequence of SEQ ID NO:85 (with X being any amino acid) as follows:

(79-104X)GLNRGL(40-42X)YSSAFS(19X)TLGQVFQRID(4X)DFDNI(16X)
TATLAHKFE(59-60X)TRGDR(X)ELRVFVXX

The information from Table 6 can be combined to define the receptor of the present invention as having an amino acid sequence of SEQ ID NO:86 as follows (with X being any amino acid and 2, 3, 4, 5, and 6 having the same definitions as for Table 6):

(79-104X)GLNRGL(12X)AA42LE(14-16X)LQG4W4L6YSSAFS(X)R3LGG(7X)
GRL6P6TLGQVFQRID6(3X)DFDNI(16X)TATLAHKFE6(12X)T3VKT(31-
32X)VT56D(3X)R6TRGDRXELRVFV6X

### Example 14

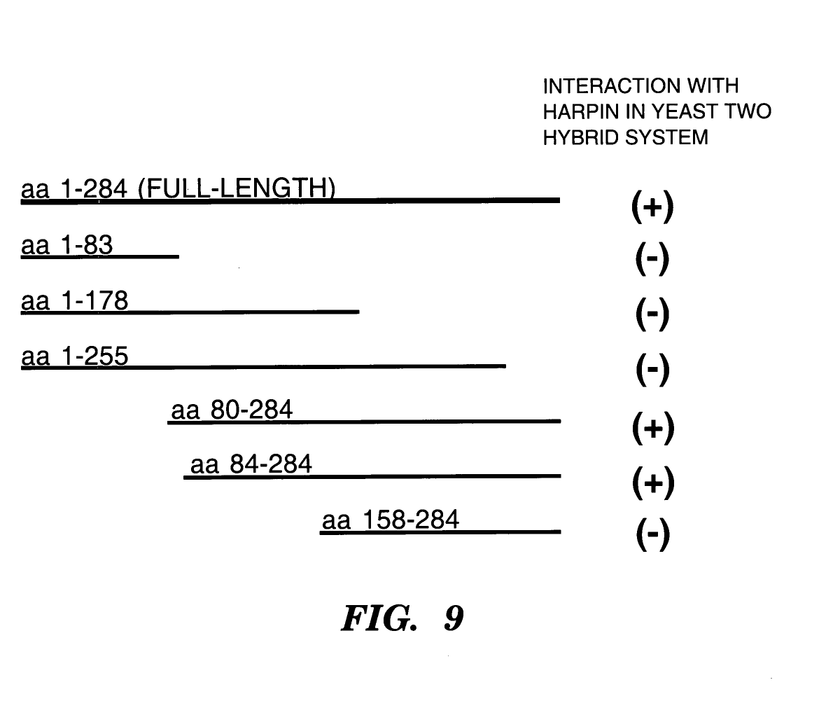

In order to further evaluate the highly conserved C-terminal region of the HrBP1p proteins and its potential role in the observed interaction between HrBP1p and harpin, AtHrBP1 deletion mutants were constructed and used in conjunction with hrpN in additional yeast-two hybrid studies. Six AtHrBP1 deletion mutants were analyzed with respect to their ability to interact with full-length harpin. The deletion mutants were cloned into the bait vector pVJL 11. Yeast strain L40 cells were then co-transformed with the AtHrBP1 deletion mutant bait constructs, and haprin cloned in the prey vector pGAD GH. The yeast-two hybrid assays were conducted including the proper controls as described above. FIG. 9 details the exact AtHrBP1p fragments analyzed, as well as the the outcome of the assays. Interaction between harpin and AtHrBP1p deletion mutant proteins was only observed with mutants containing amino acids 80-284 and 84-284. The results indicated that substantially the entire conserved region, as described earlier in Examples 13 and 14, is required for interaction between harpin and AtHrBP1p.

### Example 15

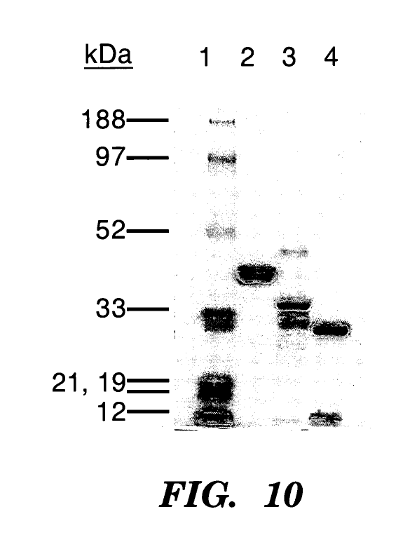

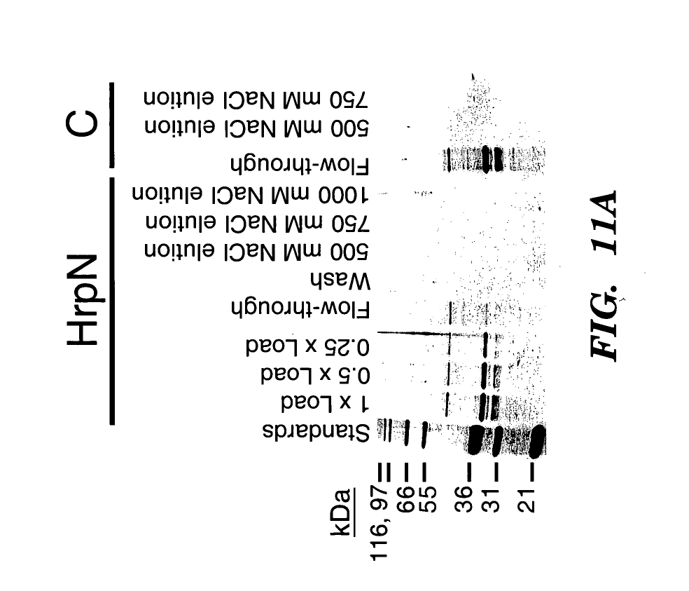

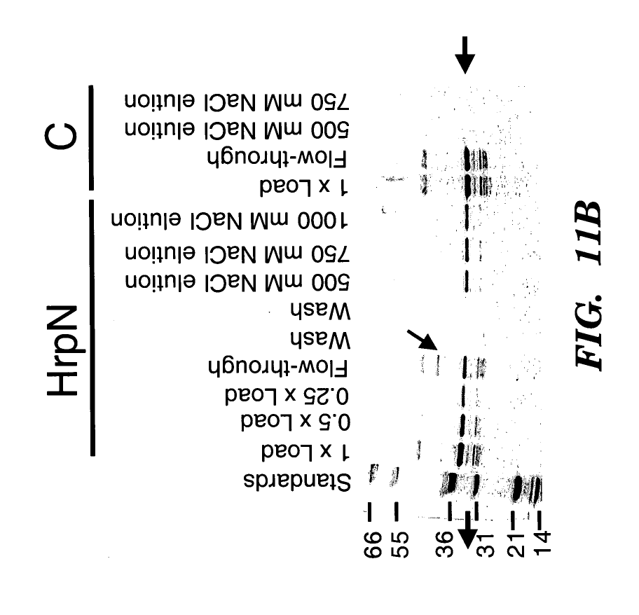

Affinity chromatography is a powerful method for characterizing and isolating components of protein complexes (Formosa et al., “Using Protein Affinity Chromatography to Probe Structure of Protein Machines”, Methods in Enzymol. 208:24-45 (1991), which is hereby incorporated by reference). Affinity chromatography was used to verify that the binding observed between AtHrBP1p and HrpN in the yeast two-hybrid assay was specific and independent of the other protein components of that assay (LexA BD, GAL4 AD). Highly purified HrpN was prepared and conjugated to agarose beads, which were then incubated with partially purified AtHrBP1p (FIG. 10, lanes 2 and 3, respectively). The unbound proteins were collected and the beads were washed extensively with binding buffer, followed by buffers with increasing concentrations of NaCl. The proteins in the fractions were separated by SDS-PAGE and visualized by silver staining. A comparison of the proteins in the load and unbound proteins in the flow-through fractions showed that nearly all the AtHrBP I p in the load was retained on the HrpN matrix (HrpN), whereas no significant binding to the mock-conjugated matrix (C) was observed (FIG. 11A). The efficiency of binding of AtHrBP1p to the HrpN matrix (>95%) and to the control matrix (<5%) was determined in replicate experiments (n=4, not shown). Very little or no AtHrBP1p eluted from the HrpN matrix when high salt buffers were applied (FIG. 11A). This suggested that the binding between HrpN and AtHrBP1p was very tight.

The experiment was repeated with CHAPS detergent (0.2% w/v) included in the binding, wash, and elution buffers. In this case, AtHrBP1p was eluted in a very pure state from the HrpN matrix using moderately high salt (FIG. 11B). Elution required at least 200 mM NaCl and was more efficient with 500-1500 mM NaCl. This result demonstrates that HrBP1p binds specifically to HrpN.

When CHAPS was included in the binding buffer, the total amount of AtHrBP1p bound to the HrpN matrix (˜75-70% bound, replicates not shown) was reduced. The CHAPS probably prevented non-specific interactions between proteins and the beads, as shown by the following observations. The inclusion of CHAPS in buffers stripped some of the HrpN from beads, causing it to appear in the flow-through. This probably accounted for some of the reduced binding by AtHrBP1p to the matrix. This effect by detergent on HrpN suggests that some HrpN was adsorbed nonspecifically to to the matrix rather than cross-linked to it. It was also observed that high salt buffers containing CHAPS eluted the tightly held AtHrBP1p that was bound to HrpN matrix in the absence of the detergent; some HrpN and small amounts of other proteins eluted with it. Trace amounts of AtHrBP1p (<5% of the load) and small amounts of other proteins also eluted from mock-conjugated beads treated this way. Therefore, the CHAPS improved the specificity of the AtHrBP1p-HrpN interaction by decreasing interactions between proteins and the agarose beads.

AtHrBP1p has a large trypsin-resistant fragment, designated TL-HrBP1p (˜25 kDa; FIG. 10, lane 4) that initiates with residue 52 of the full-length AtHrBP1p. TL-HrBP1p could also be missing residues from the C-terminus of the protein since there are 4 potential cleavage sites within the last 16 amino acids at the end. Purified TL-HrBP1p was tested for its ability to bind to HrpN matrix in the presence of CHAPS. A significant percentage (40%) of the input TL-HrBP1p was specifically retained on the HrpN matrix. This result confirms the observation made using the yeast two-hybrid assay that the C-terminal conserved region of the protein is largely responsible for its interaction with HrpN. Residues missing from TL-HrBP1p as a result of the proteolysis might normally contribute to the strength of the interaction between AtHrBP1p and HrpN.

### Example 16

A transgenic approach was used for functional analysis of AtHrBP1p. Anti-sense AtHrBP1, which is complementary to SEQ ID NO:2, was sub-cloned into binary vector pPZP212, and is under the control of the NOS promoter. Arabidopsis thaliana plants were transformed with this construct via an Agrobacteria mediated method. The Agrobacterium tumefaciens strain used was GV3101 (C58C1 Rifr) pMP90 (Gmr). These antisense lines were designated “as” lines.

Arabidopsis plants were also transformed with a construct, which has an inverted repeat with a sense strand of AtHrBP1 coding region bases 4-650 (i.e. bases 20-666 of SEQ ID NO:2) and the complementary sequence of bases 20-516 of AtHrBP1 cDNA (i.e. SEQ ID NO:2). This construct generated a double-stranded mRNA in transformed plants. These transgenic lines were designated “d” lines.

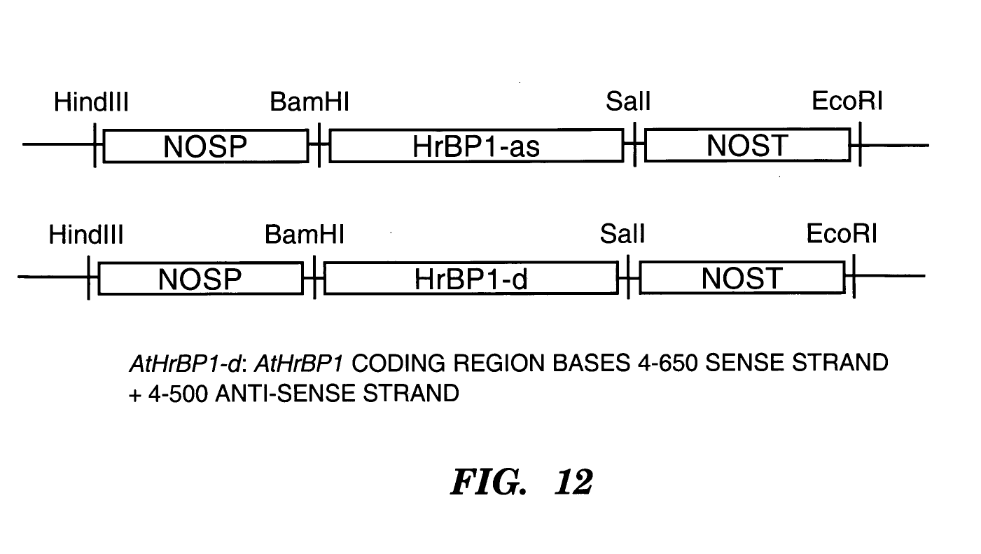

FIG. 12 shows the constructs used to transform Arabidopsis.

Both antisense and double-stranded approaches were to silence the expression of AtHrBP1. The double stranded RNA method was found to be more efficient in silencing the AtHrBP1 gene. Some transgenic Arabidopsis lines showed spontaneous HR-mimic lesion. The most severe line was developmentally retarded, looked very unhealthy, and did not produce seeds. The transgenic and control Arabidopsis thaliana Columbia plants were grown in autoclaved potting mix in a controlled environment room at a day and night temperature of 23-20° C. and a photoperiod of 14 h light.

### Example 17

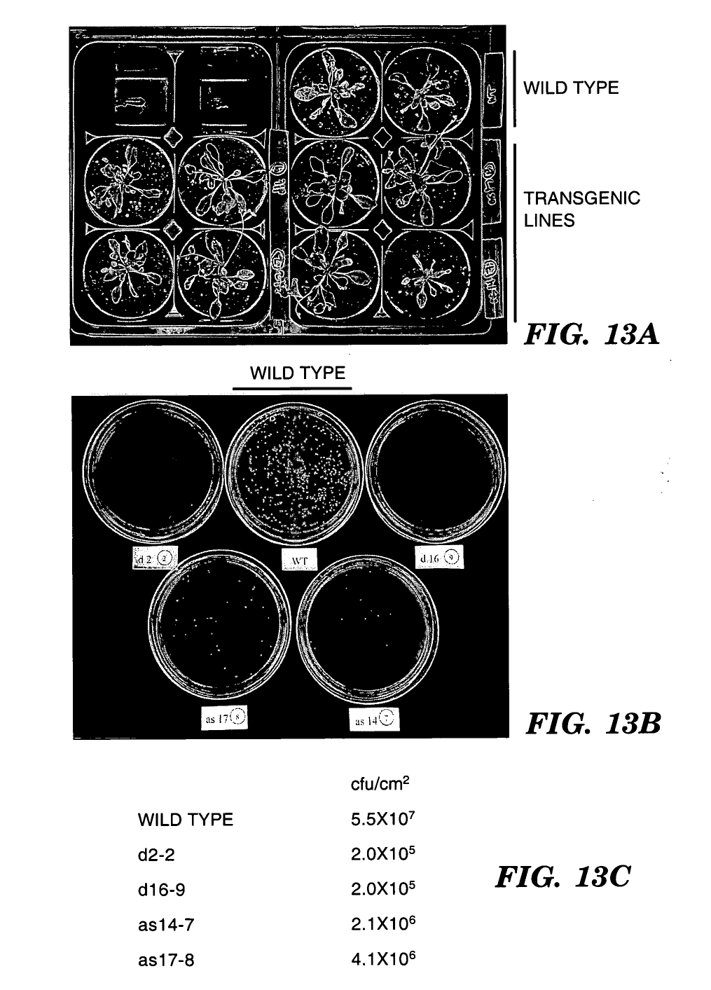

Plants were grown in autoclaved potting mix in a controlled environment room with a day and night temperature of 23-20° C. and a photoperiod of 14 h light. 25-day-old plants were inoculated with Pseudomonas syringae p.v. tomato DC3000 by dipping the above soil parts of the plants in 10⁸ cells ml⁻¹ bacteria suspension for 10 second. Seven days after DC3000 inoculation, leaf disks were harvested with a cork borer. Bacteria were extracted from leaf disks in 10 mM MgCl₂ and plated on King's B agar containing 100 μg rifampicin/ml. Plates were incubated at 28° C. for 2 days (FIG. 13B) and colonies were counted. In FIG. 13A, wild type Arabidopsis plants had significantly more disease development than transgenic plants. Bacteria counting (FIG. 13C) showed that transgenic plants had at least one order of magnitude less of DC3000 growing inside the leaves. AtHrBP1p appeared to function like a negative regulator of plant defense signal transduction pathway in Arabidopsis. Its silencing imparted plants with the ability to resist Pseudomonas syringae p.v. tomato DC3000.

### Example 18

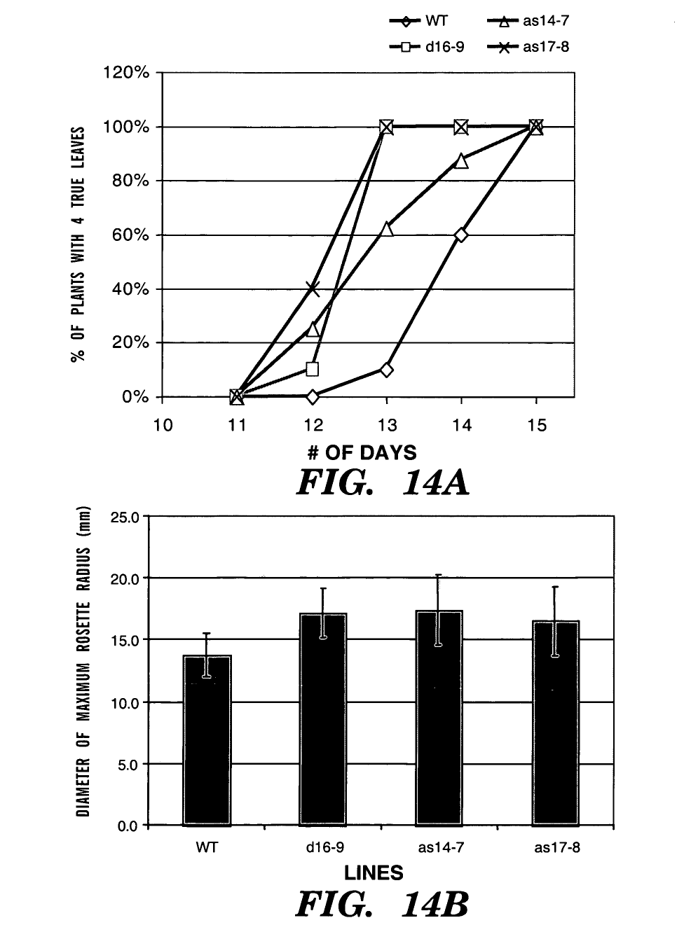

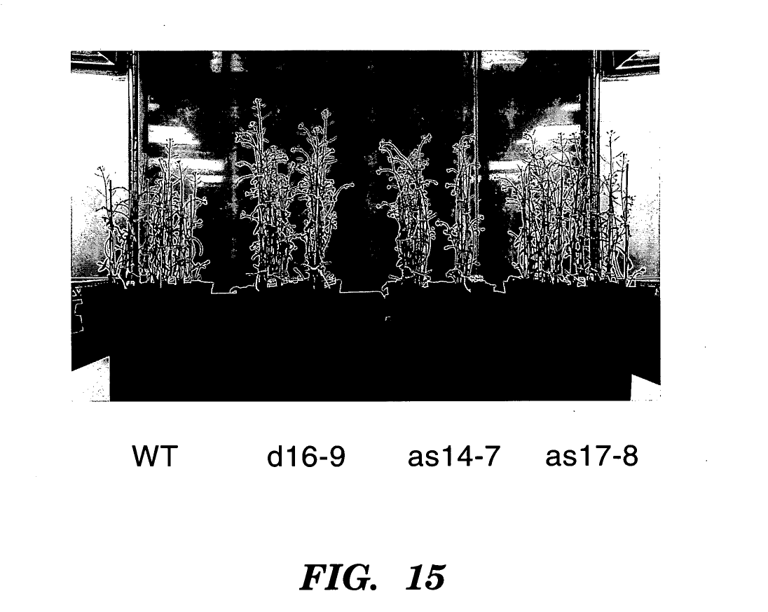

Wild type Col-0 Arabidopsis plants and three independent AtHrBP1 suppression lines were grown in soil mix 1:1:1 (Sunshine LC1: perlite: coarse vermiculite) in a controlled environment room with a day and night temperature of 23-20° C. and photoperiod of 16 hour light. The suppression lines progressed to different growth/developmental stages faster than wild type plants. In comparing plants at the same growth stage, the suppression lines were larger than wild type plants. FIG. 14A shows data evaluating the percentage of plants with 4 true leaves >1 mm in length at sequential days after sowing. As shown, most suppression lines grew to this stage 2 days earlier than wild type plants. FIG. 14B details data regarding the diameter of maximum rosette radius achieved by wild type and suppression lines. Measurements were made on different days once plants entered the four-true-leaf stage. FIG. 15 depicts a visual difference between wild type and suppression lines 32 days after sowing. Stems of the AtHrBP1 transgenic plants were more elongated than those of the wild type plants.

### Example 19

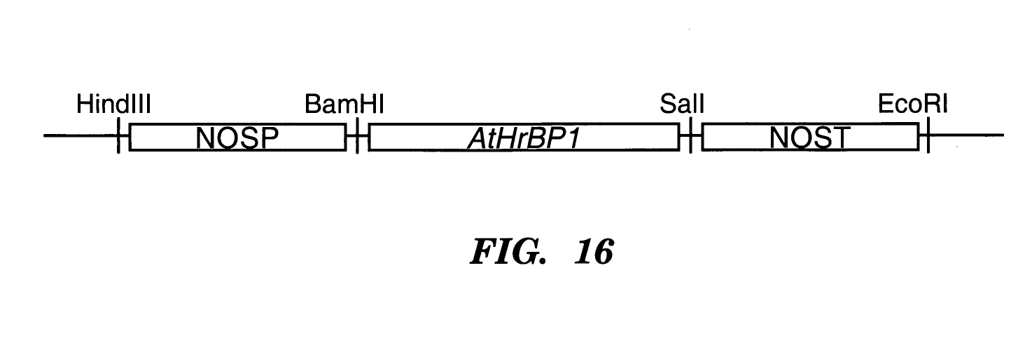

The AtHrBP1 coding region, bases 17-871 of SEQ ID NO:2, was subcloned into binary vector pPZP212 and was under the control of the NOS promoter (see FIG. 16). Tobacco plants were transformed with this construct via an Agrobacteria mediated method. The Agrobacterium tumefaciens strain used was LBA4404.

### Example 20

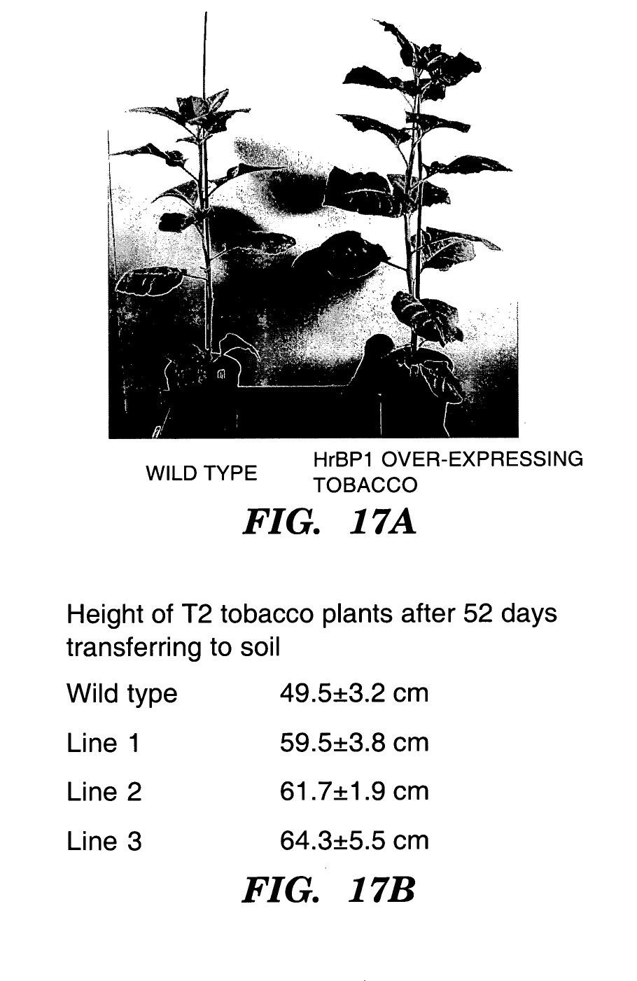

AtHrBP1p was overexpressed in tobacco plants under the control of the NOS promoter. FIG. 16 shows the construct used for tobacco transformation. Three high expression lines were chosen for further studies in the T2 generation. The AtHrBP1p-overexpressing lines were about 20-30% taller than wild type Xanthi AN plants (see FIG. 17). When infiltrated with purified harpin, the transgenic lines developed HR much faster than wild type plants. This is consistent with another experiment in which purified recombinant His-tagged AtHrBP1p, when co-infiltrated along with purified harpin, increased the sensitivity of tobacco plants to the harpin protein.

### Example 21

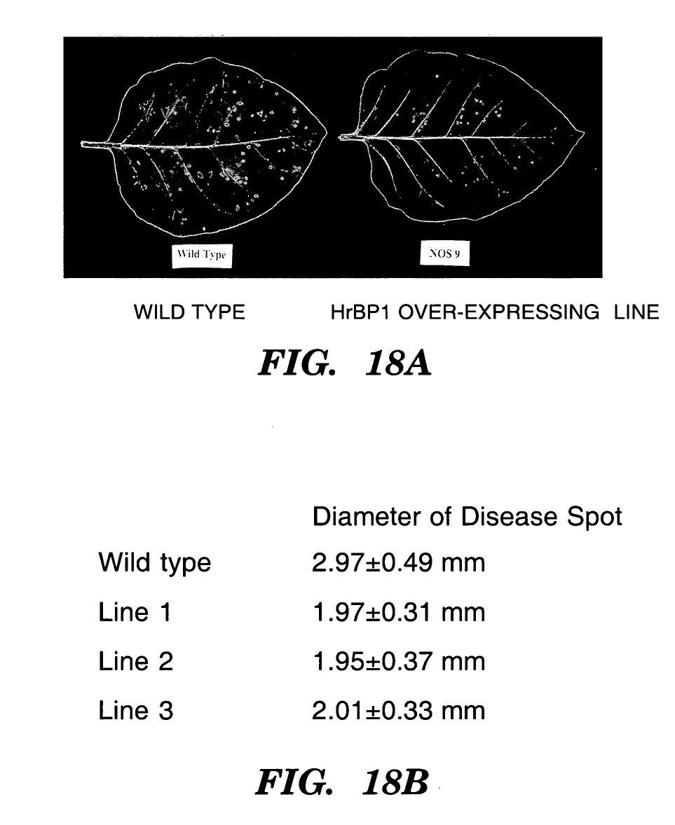

61 -day-old wild type and AtHrBP1p-overexpressing Xanthi NN tobacco plants were inoculated with tobacco mosaic virus by rubbing tobacco mosaic virus (TMV) with diatomaceous earth on the upper surface of leaves. Lesions appeared 2 days after manual inoculation. The picture in FIG. 1 8A was taken 3 days after inoculation. The diameter of disease spots was measured. On average, the diameter of a lesion on leaves of transgenic plants was 33.4% less than that seen on wild type plants (FIG. 18B). Therefore, the surface area of lesions on transgenic plant leaves was about 44.3% of those of the wild type plants.

### Example 22

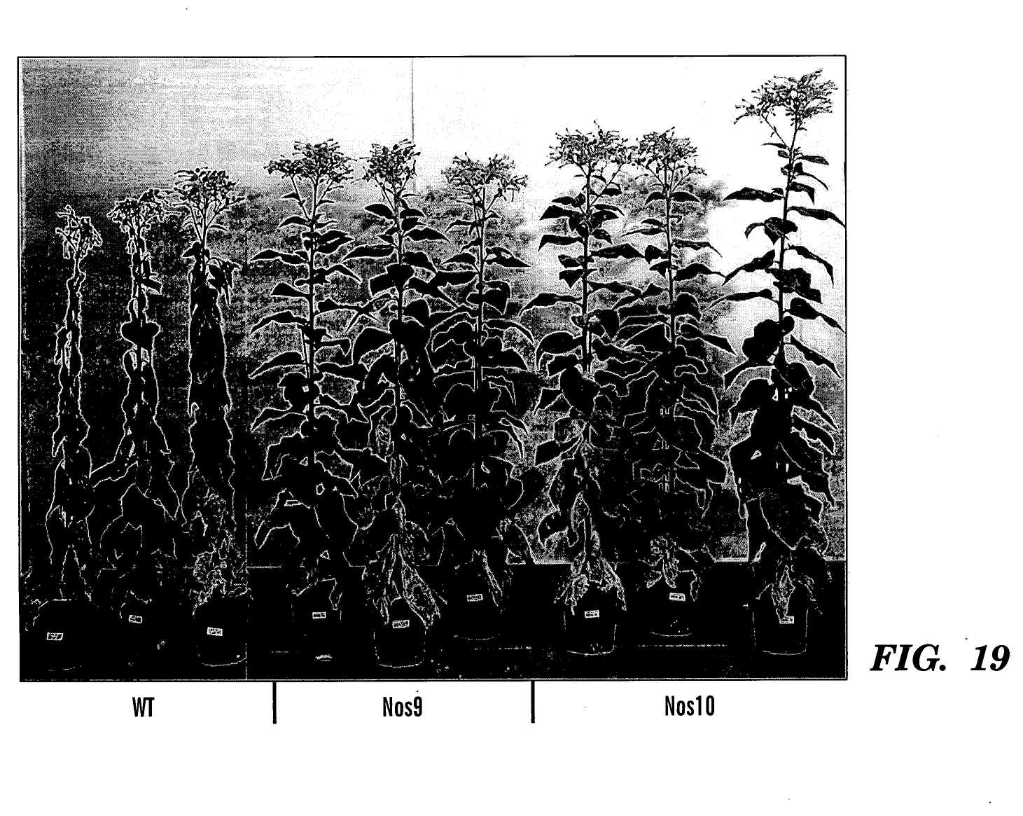

52-day-old wild type and two independent AtHrBP1p-overexpressing Xanthi NN tobacco plants were inoculated with Pseudomonas solanacearum by root cutting. Disease symptoms started 11 days after inoculation. Diseases symptoms in wild type plants progressed through the course of the study. However, as seen in FIG. 19, the transgenic lines remained relatively healthy. FIG. 19 shows representative wild type and AtHrBP1p-overexpressing transgenic line plants 44 days after Pseudomonas inoculation.

Although the invention has been described in detail for the purpose of illustration, it is understood that such details are solely for that purpose. The variations can be made therein by those skilled in the art without departing from the spirit of the scope of the invention which is defined by the following claims.

1. An isolated protein which serves as a receptor in plants for plant pathogen hypersensitive response elicitors.

2. A protein according to claim 1, wherein the plant pathogen is selected from the group consisting of Erwinia, Pseudomonas, Xanthamonas, Phytophthora, and Clavibacter.

3. A protein according to claim 2, wherein the plant pathogen is an Erwinia pathogen.

4. A protein according to claim 3, wherein the plant pathogen is Erwinia amylovora.

5. A protein according to claim 1, wherein the protein is from a monocot.

6. A protein according to claim 5, wherein the protein is from rice, barley, wheat, or maize.

7. A protein according to claim 1, wherein the protein comprises an amino acid sequence of SEQ. ID. Nos. 4, 10, 14, 16, 20, 22, or 24.

8. A protein according to claim 1, wherein the protein is from a dicot.

9. A protein according to claim 8, wherein the protein is from Arabidopsis thaliana, cotton, soybean, tomato, potato, grapefruit, apple, tobacco, or grape.

10. A protein according to claim 1, wherein the protein has an amino acid sequence of SEQ. ID. Nos. 1, 6, 8, 12, 18, 26, 28, 30, 32, or 34.

11. A protein according to claim 1, wherein the protein is recombinant.

12. A protein according to claim 1, wherein the protein has an amino acid motif selected from the group consisting of the motif of SEQ ID. No. 69, the motif of SEQ ID. No. 70, the motif of SEQ ID. No. 71, the motif of SEQ ID. No. 72, the motif of SEQ ID. No. 73, the motif of SEQ ID. No. 74, the motif of SEQ ID. No. 75, and combinations thereof.

13. A protein according to claim 1, wherein the protein has an amino acid motif selected from the group consisting of the motif of SEQ ID. No. 76, the motif of SEQ ID. No. 77, the motif of SEQ ID. No. 78, the motif of SEQ ID. No. 79, the motif of SEQ ID. No. 80, the motif of SEQ ID. No. 81, the motif of SEQ ID. No. 82, the motif of SEQ ID. No. 83, the motif of SEQ ID. No. 84, and combinations thereof.

14. A protein according to claim 1, wherein the protein has an amino acid sequence of SEQ ID. No. 85.

15. A protein according to claim 1, wherein the protein has an amino acid sequence of SEQ ID. No. 86.

16. An isolated nucleic acid molecule encoding a protein according to claim 1.

17. A nucleic acid molecule according to claim 16, wherein the plant pathogen is selected from the group consisting of Erwinia, Pseudomonas, Xanthamonas, Phytophthora, and Clavibacter.

18. A nucleic acid molecule according to claim 17, wherein the plant pathogen is an Erwinia pathogen.

19. A nucleic acid molecule according to claim 18, wherein the plant pathogen is Erwinia amylovora.

20. A nucleic acid molecule according to claim 16, wherein the protein is from a monocot.

21. A nucleic acid molecule according to claim 20, wherein the protein is from rice, barley, wheat, or maize.

22. A nucleic acid molecule according to claim 16, wherein the protein comprises an amino acid sequence of SEQ. ID. Nos. 4, 10, 14, 16, 20, 22, or 24.

23. A nucleic acid molecule according to claim 16, wherein the nucleic acid hybridizes to the nucleotide sequence of SEQ. ID. Nos. 5, 11, 15, 17, 21, 23, or 25 under stringent conditions of hybridization in buffer comprising 20% formamide in 0.9 M saline/0.09M SSC at a temperature of 42° C.

24. A nucleic acid molecule according to claim 16, wherein the nucleic acid has a nucleotide sequence comprising SEQ. ID. Nos. 5, 11, 15, 17, 21, 23, or 25.

25. A nucleic acid molecule according to claim 16, wherein the protein is from a dicot.

26. A nucleic acid molecule according to claim 25, wherein the protein is from Arabidopsis thaliana, cotton, soybean, tomato, potato, grapefruit, apple, tobacco, or grape.

27. A nucleic acid molecule according to claim 25, wherein the protein has an amino acid sequence of SEQ. ID. Nos. 1, 6, 8, 12, 18, 26, 28, 30, 32, or 34.

28. A nucleic acid molecule according to claim 25, wherein the nucleic acid hybridizes to the nucleotide sequence of SEQ. ID. Nos. 2, 7, 9, 13, 19, 27, 29, 31, 33, 35, or 39 under stringent conditions of hybridization in buffer comprising 20% formamide in 0.9M saline/0.09M SSC at a temperature of 42° C.

29. A nucleic acid molecule according to claim 25, wherein the nucleic acid has a nucleotide sequence of SEQ. ID. Nos. 2, 7, 9, 13, 19, 27, 29, 31, 33, or 35.

30. A nucleic acid according to claim 25, wherein the nucleic acid hybridizes to a nucleotide sequence of SEQ. ID. No. 3 under stringent conditions of hybridization in buffer comprising 20% formamide in 0.9M saline/0.09M SSC buffer at a temperature of 42° C.

31. A nucleic acid according to claim 25, wherein the nucleic acid has a nucleotide sequence comprising SEQ. ID. No. 3.

32. A nucleic acid according to claim 16, wherein the protein has an amino acid motif selected from the group consisting of the motif of SEQ ID. No. 69, the motif of SEQ ID. No. 70, the motif of SEQ ID. No. 71, the motif of SEQ ID. No. 72, the motif of SEQ ID. No. 73, the motif of SEQ ID. No. 74, the motif of SEQ ID. No. 75, and combinations thereof.

33. A nucleic acid according to claim 16, wherein the protein has an amino acid motif selected from the group consisting of the motif of SEQ ID. No. 76, the motif of SEQ ID. No. 77, the motif of SEQ ID. No. 78, the motif of SEQ ID. No. 79, the motif of SEQ ID. No. 80, the motif of SEQ ID. No. 81, the motif of SEQ ID. No. 82, the motif of SEQ ID. No. 83, the motif of SEQ ID. No. 84, and combinations thereof.

34. A nucleic acid according to claim 16, wherein the protein has an amino acid sequence of SEQ ID. No. 85.

35. A nucleic acid according to claim 16, wherein the protein has an amino acid sequence of SEQ ID. No. 86.

36. An antisense nucleic acid molecule to the nucleic acid according to claim 16.

37. An expression vector containing a nucleic acid molecule according to claim 16 which is heterologous to the expression vector.

38. An expression vector according to claim 37, wherein the nucleic acid molecule is positioned in the expression vector in sense orientation and correct reading frame.

39. An expression vector according to claim 37, wherein either: (1) the protein has an amino acid sequence of SEQ. ID. Nos. 1, 6, 8, 12, 18, 26, 28, 30, 32, or 34; (2) the nucleic acid hybridizes to a nucleotide sequence of SEQ. ID. Nos. 2, 7, 9, 13, 19, 27, 29, 31, 33, 35, or 39 under stringent conditions of hybridization in buffer comprising 20% formamide in 0.9M saline/0.09M SSC at a temperature of 42° C.; (3) the nucleic acid comprises a nucleotide sequence of SEQ. ID. Nos. 2, 7, 9, 13, 19, 27, 29, 31, 33, or 35; (4) the nucleic acid hybridizes to a nucleotide sequence of SEQ. ID. No.3 under stringent conditions of hybridization in buffer comprising 20% formamide in 0.9M saline/0.09M SSC at a temperature of 42° C.; (5) the nucleic acid comprises a nucleotide sequence of SEQ. ID. No.3; (6) the protein has an amino acid sequence of SEQ. ID. Nos. 4, 10, 14, 16, 20, 22, or 24; (7) the nucleic acid hybridizes to the nucleotide sequence of SEQ. ID. Nos. 5, 11, 15, 17, 21, 23, or 25 under stringent conditions of hybridization in buffer comprising 20% formamide in 0.9 M saline/0.09M SSC at a temperature of 42° C., (8) the nucleic acid comprises a nucleotide sequence of SEQ. ID. Nos. 5, 11, 15, 17, 21, 23, or 25; (9) the protein has an amino acid motif selected from the group consisting of the motif of SEQ ID. No. 69, the motif of SEQ ID. No. 70, the motif of SEQ ID. No. 71, the motif of SEQ ID. No. 72, the motif of SEQ ID. No. 73, the motif of SEQ ID. No. 74, the motif of SEQ ID. No. 75, and combinations thereof; (10) the protein has an amino acid motif selected from the group consisting of the motif of SEQ ID. No. 76, the motif of SEQ ID. No. 77, the motif of SEQ ID. No. 78, the motif of SEQ ID. No. 79, the motif of SEQ ID. No. 80, the motif of SEQ ID. No. 81, the motif of SEQ ID. No. 82, the motif of SEQ ID. No. 83, the motif of SEQ ID. No. 84, and combinations thereof; (11) the protein has an amino acid sequence of SEQ ID. No. 85; or (12) the protein has an amino acid sequence of SEQ ID. No. 86.

40. An expression vector containing a nucleic acid molecule according to claim 36 which is heterologous to the expression vector.

41. A transgenic host cell transformed with the nucleic acid molecule according to claim 16.

42. A host cell transformed according to claim 41, wherein the host cell is selected from the group consisting of a plant cell, a bacterial cell, and a fungal cell.

43. A host cell according to claim 41, wherein the DNA molecule is transformed with an expression system.

44. A host cell according to claim 41, wherein either: (1) the protein has an amino acid sequence of SEQ. ID. Nos. 1, 6, 8, 12, 18, 26, 28, 30, 32, or 34; (2) the nucleic acid hybridizes to a nucleotide sequence of SEQ. ID. Nos. 2, 7, 9, 13, 19, 27, 29, 31, 33, 35, or 39 under stringent conditions of hybridization in buffer comprising 20% formamide in 0.9M saline/0.09M SSC at a temperature of 42° C.; (3) the nucleic acid comprises a nucleotide sequence of SEQ. ID. Nos. 2, 7, 9, 13, 19, 27, 29, 31, 33, or 35; (4) the nucleic acid hybridizes to a nucleotide sequence of SEQ. ID. No. 3 under stringent conditions of hybridization in buffer comprising 20% formamide in 0.9M saline/0.09M SSC at a temperature of 42° C.; (5) the nucleic acid comprises a nucleotide sequence of SEQ. ID. No. 3; (6) the protein has an amino acid sequence of SEQ. ID. Nos. 4, 10, 14, 16, 20, 22, or 24; (7) the nucleic acid hybridizes to the nucleotide sequence of SEQ. ID. Nos. 5, 11, 15, 17, 21, 23, or 25 under stringent conditions of hybridization in buffer comprising 20% formamide in 0.9 M saline/0.09M SSC at a temperature of 42° C.; (8) the nucleic acid comprises a nucleotide sequence of SEQ. ID. Nos. 5, 11, 15, 17, 21, 23, or 25; (9) the protein has an amino acid motif selected from the group consisting of the motif of SEQ ID. No. 69, the motif of SEQ ID. No. 70, the motif of SEQ ID. No. 71, the motif of SEQ ID. No. 72, the motif of SEQ ID. No. 73, the motif of SEQ ID. No. 74, the motif of SEQ ID. No. 75, and combinations thereof; (10) the protein has an amino acid motif selected from the group consisting of the motif of SEQ ID. No. 76, the motif of SEQ ID. No. 77, the motif of SEQ ID. No. 78, the motif of SEQ ID. No. 79, the motif of SEQ ID. No. 80, the motif of SEQ ID. No. 81, the motif of SEQ ID. No. 82, the motif of SEQ ID. No. 83, the motif of SEQ ID. No. 84, and combinations thereof; (11) the protein has an amino acid sequence of SEQ ID. No. 85; or (12) the protein has an amino acid sequence of SEQ ID. No. 86.

45. A host cell transformed with a nucleic acid molecule according to claim 36.

46. A transgenic plant transformed with the DNA molecule of claim 16.

47. A transgenic plant according to claim 46, wherein the plant is selected from the group consisting of alfalfa, rice, wheat, barley, rye, cotton, sunflower, peanut, corn, potato, sweet potato, bean, pea, chicory, lettuce, endive, cabbage, brussel sprout, beet, parsnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.

48. A transgenic plant according to claim 46, wherein the plant is selected from the group consisting of Arabidopsis thaliana, Saintpaulia, petunia, pelargonium, poinsettia, chrysanthemum, carnation, and zinnia.

49. A transgenic plant according to claim 46, wherein the plant is a monocot.

50. A transgenic plant according to claim 46, wherein the plant is from a dicot.

51. A transgenic plant according to claim 46, wherein either: (1) the protein has an amino acid sequence of SEQ. ID. Nos. 1, 6, 8, 12, 18, 26, 28, 30, 32, or 34; (2) the nucleic acid hybridizes to a nucleotide sequence of SEQ. ID. Nos. 2, 7, 9, 13, 19, 27, 29, 31, 33, 35, or 39 under stringent conditions of hybridization in buffer comprising 20% formamide in 0.9M saline/0.09M SSC at a temperature of 42° C.; (3) the nucleic acid comprises a nucleotide sequence of SEQ. ID. Nos. 2, 7, 9, 13, 19, 27, 29, 31, 33, or 35; (4) the nucleic acid hybridizes to a nucleotide sequence of SEQ. ID. No. 3 under stringent conditions of hybridization in buffer comprising 20% formamide in 0.9M saline/0.09M SSC at a temperature of 42° C.; (5) the nucleic acid comprises a nucleotide sequence of SEQ. ID. No. 3; (6) the protein has an amino acid sequence of SEQ. ID. Nos. 4, 10, 14, 16, 20, 22, or 24; (7) the nucleic acid hybridizes to the nucleotide sequence of SEQ. ID. Nos. 5, 11, 15, 17, 21, 23, or 25 under stringent conditions of hybridization in buffer comprising 20% formamide in 0.9 M saline/0.09M SSC at a temperature of 42° C.; (8) the nucleic acid comprises a nucleotide sequence of SEQ. ID. Nos. 5, 11, 15, 17, 21, 23, or 25; (9) the protein has an amino acid motif selected from the group consisting of the motif of SEQ ID. No. 69, the motif of SEQ ID. No. 70, the motif of SEQ ID. No. 71, the motif of SEQ ID. No. 72, the motif of SEQ ID. No. 73, the motif of SEQ ID. No. 74, the motif of SEQ ID. No. 75, and combinations thereof; (10) the protein has an amino acid motif selected from the group consisting of the the motif of SEQ ID. No. 76, motif of SEQ ID. No. 77, the motif of SEQ ID. No. 78, the motif of SEQ ID. No. 79, the motif of SEQ ID. No. 80, the motif of SEQ ID. No. 81, the motif of SEQ ID. No. 82, the motif of SEQ ID. No. 83, the motif of SEQ ID. No. 84, and combinations thereof; (11) the protein has an amino acid sequence of SEQ ID. No. 85; or (12) the protein has an amino acid sequence of SEQ ID. No. 86.

52. A transgenic plant transformed with a nucleic acid molecule according to claim 36.

53. A transgenic plant seed transformed with the DNA molecule of claim 16.

54. A transgenic plant seed according to claim 53, wherein the plant is selected from the group consisting of alfalfa, rice, wheat, barley, rye, cotton, sunflower, peanut, corn, potato, sweet potato, bean, pea, chicory, lettuce, endive, cabbage, brussel sprout, beet, parsnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.

55. A transgenic plant seed according to claim 53, wherein the plant is selected from the group consisting of Arabidopsis thaliana, Saintpaulia, petunia, pelargonium, poinsettia, chrysanthemum, carnation, and zinnia.

56. A transgenic plant seed according to claim 53, wherein the plant is a monocot.

57. A transgenic plant seed according to claim 53, wherein the plant is a dicot.

58. A transgenic plant seed according to claim 53, wherein either: (1) the protein has an amino acid sequence of SEQ. ID. Nos. 1, 6, 8, 12, 18, 26, 28, 30, 32, or 34; (2) the nucleic acid hybridizes to a nucleotide sequence of SEQ. ID. Nos. 2, 7, 9, 13, 19, 27, 29, 31, 33, 35, or 39 under stringent conditions of hybridization in buffer comprising 20% formamide in 0.9M saline/0.09M SSC at a temperature of 42° C.; (3) the nucleic acid comprises a nucleotide sequence of SEQ. ID. Nos. 2, 7, 9, 13, 19, 27, 29, 31, 33, or 35; (4) the nucleic acid hybridizes to a nucleotide sequence of SEQ. ID. No. 3 under stringent conditions of hybridization in buffer comprising 20% formamide in 0.9M saline/0.09M SSC at a temperature of 42° C.; (5) the nucleic acid comprises a nucleotide sequence of SEQ. ID. No. 3; (6) the protein has an amino acid sequence of SEQ. ID. Nos. 4, 10, 14, 16, 20, 22, or 24; (7) the nucleic acid hybridizes to the nucleotide sequence of SEQ. ID. Nos. 5, 11, 15, 17, 21, 23, or 25 under stringent conditions of hybridization in buffer comprising 20% formamide in 0.9 M saline/0.09M SSC at a temperature of 42° C.; (8) the nucleic acid comprises a nucleotide sequence of SEQ. ID. Nos. 5, 11, 15, 17, 21, 23, or 25; (9) the protein has an amino acid motif selected from the group consisting of the motif of SEQ ID. No. 69, the motif of SEQ ID. No. 70, the motif of SEQ ID. No. 71, the motif of SEQ ID. No. 72, the motif of SEQ ID. No. 73, the motif of SEQ ID. No. 74, the motif of SEQ ID. No. 75, and combinations thereof; (10) the protein has an amino acid motif selected from the group consisting of the motif of SEQ ID. No. 76, the motif of SEQ ID. No. 77, the motif of SEQ ID. No. 78, the motif of SEQ ID. No. 79, the motif of SEQ ID. No. 80, the motif of SEQ ID. No. 81, the motif of SEQ ID. No. 82, the motif of SEQ ID. No. 83, the motif of SEQ ID. No. 84, and combinations thereof; (11) the protein has an amino acid sequence of SEQ ID. No. 85; or (12) the protein has an amino acid sequence of SEQ ID. No. 86.

59. A transgenic plant seed transformed with a nucleic acid molecule according to claim 36.

60. A method of identifying agents targeting plant cells comprising:
forming a reaction mixture comprising a protein according to claim 1 and a candidate agent;
evaluating the reaction mixture for binding between the protein and the candidate agent; and
identifying candidate compounds which bind to the protein in the reaction mixture as plant cell targeting agents.

61. A method according to claim 60, wherein the protein is from a monocot.

62. A method according to claim 60, wherein the protein is from rice, barley, wheat, or maize.

63. A method according to claim 60, wherein the protein has an amino acid sequence comprises SEQ. ID. Nos. 4, 10, 14, 16, 20, 22, or 24.

64. A method according to claim 60, wherein the protein is from a dicot.

65. A method according to claim 64, wherein the protein is from Arabidopsis thaliana, cotton, soybean, tomato, potato, grapefruit, apple, tobacco, or grape.

66. A method according to claim 60, wherein the protein has an amino acid sequence of SEQ. ID. Nos. 1, 6, 8, 12, 18, 26, 28, 30, 32, or 34.

67. A method according to claim 60, wherein the protein has an amino acid motif selected from the group consisting of the motif of SEQ ID. No. 69, the motif of SEQ ID. No. 70, the motif of SEQ ID. No. 71, the motif of SEQ ID. No. 72, the motif of SEQ ID. No. 73, the motif of SEQ ID. No. 74, the motif of SEQ ID. No. 75, and combinations thereof.

68. A method according to claim 60, wherein the protein has an amino acid motif selected from the group consisting of the motif of SEQ ID. No. 76, the motif of SEQ ID. No. 77, the motif of SEQ ID. No. 78, the motif of SEQ ID. No. 79, the motif of SEQ ID. No. 80, the motif of SEQ ID. No. 81, the motif of SEQ ID. No. 82, the motif of SEQ ID. No. 83, the motif of SEQ ID. No. 84, and combinations thereof.

69. A method according to claim 60, wherein the protein has an amino acid sequence of SEQ ID. No. 85.

70. A method according to claim 60, wherein the protein has an amino acid sequence of SEQ ID. No. 86.

71. A method of identifying agents targeting plant cells comprising:
forming a reaction mixture comprising a host cell transformed with a nucleic acid molecule according to claim 16 and a candidate agent;
evaluating the reaction mixture for binding between protein produced by the host cell and the candidate agent; and
identifying candidate compounds which bind to the protein produced by the host cell in the reaction mixture as plant cell targeting agents.

72. A method according to claim 71, wherein the protein is from a monocot.

73. A method according to claim 72, wherein the protein is from rice, barley, wheat, or maize.

74. A method according to claim 71, wherein the protein is from a dicot.

75. A method according to claim 71, wherein the protein is from Arabidopsis thaliana, cotton, soybean, tomato, potato, grapefruit, apple, tobacco, or grape.

76. A method according to claim 71, wherein either: (1) the protein has an amino acid sequence of SEQ. ID. Nos. 1, 6, 8, 12, 18, 26, 28, 30, 32, or 34; (2) the nucleic acid hybridizes to a nucleotide sequence of SEQ. ID. Nos. 2, 7, 9, 13, 19, 27, 29, 31, 33, 35, or 39 under stringent conditions of hybridization in buffer comprising 20% formamide in 0.9M saline/0.09M SSC at a temperature of 42° C.; (3) the nucleic acid comprises a nucleotide sequence of SEQ. ID. Nos. 2, 7, 9, 13, 19, 27, 29, 31, 33, or 35; (4) the nucleic acid hybridizes to a nucleotide sequence of SEQ. ID. No. 3 under stringent conditions of hybridization in buffer comprising 20% formamide in 0.9M saline/0.09M SSC at a temperature of 42° C.; (5) the nucleic acid comprises a nucleotide sequence of SEQ. ID. No. 3; (6) the protein has an amino acid sequence of SEQ. ID. Nos. 4, 10, 14, 16, 20, 22, or 24; (7) the nucleic acid hybridizes to the nucleotide sequence of SEQ. ID. Nos. 5, 11, 15, 17, 21, 23, or 25 under stringent conditions of hybridization buffer comprising 20% formamide in 0.9 M saline/0.09M SSC at a temperature of 42° C.; or (8) the nucleic acid comprises a nucleotide sequence of SEQ. ID. Nos. 5, 11, 15, 17, 21, 23, or 25.

77. A method according to claim 71, wherein the protein has an amino acid motif selected from the group consisting of the motif of SEQ ID. No. 69, the motif of SEQ ID. No. 70, the motif of SEQ ID. No. 71, the motif of SEQ ID. No. 72, the motif of SEQ ID. No. 73, the motif of SEQ ID. No. 74, the motif of SEQ ID. No. 75, and combinations thereof.

78. A method according to claim 71, wherein the protein has an amino acid motif selected from the group consisting of the motif of SEQ ID. No. 76, the motif of SEQ ID. No. 77, the motif of SEQ ID. No. 78, the motif of SEQ ID. No. 79, the motif of SEQ ID. No. 80, the motif of SEQ ID. No. 81, the motif of SEQ ID. No. 82, the motif of SEQ ID. No. 83, the motif of SEQ ID. No. 84, and combinations thereof.

79. A method according to claim 71, wherein the protein has an amino acid sequence of SEQ ID. No. 85.

80. A method according to claim 71, wherein the protein has an amino acid sequence of SEQ ID. No. 86.

81. A method of enhancing plant receptivity to treatment with hypersensitive response elicitors comprising:
providing a transgenic plant or transgenic plant seed transformed with the nucleic acid molecule according to claim 16.

82. A method according to claim 81, wherein either: (1) the protein has an amino acid sequence of SEQ. ID. Nos. 1, 6, 8, 12, 18, 26, 28, 30, 32, or 34; (2) the nucleic acid hybridizes to a nucleotide sequence of SEQ. ID. Nos. 2, 7, 9, 13, 19, 27, 29, 31, 33, 35, or 39 under stringent conditions of hybridization in buffer comprising 20% formamide in 0.9M saline/0.09M SSC at a temperature of 42° C.; (3) the nucleic acid comprises a nucleotide sequence of SEQ. ID. Nos. 2, 7, 9, 13, 19, 27, 29, 31, 33, or 35; (4) the nucleic acid hybridizes to a nucleotide sequence of SEQ. ID. No. 3 under stringent conditions of hybridization in buffer comprising 20% formamide in 0.9M saline/0.09M SSC at a temperature of 42° C.; (5) the nucleic acid comprises a nucleotide sequence of SEQ. ID. No. 3; (6) the protein has an amino acid sequence of SEQ. ID. Nos. 4, 10, 14, 16, 20, 22, or 24; (7) the nucleic acid hybridizes to the nucleotide sequence of SEQ. ID. Nos. 5, 11, 15, 17, 21, 23, or 25 under stringent conditions of hybridization in buffer comprising 20% formamide in 0.9 M saline/0.09M SSC at a temperature of 42° C.; or (8) the nucleic acid comprises a nucleotide sequence of SEQ. ID. Nos. 5, 11, 15, 17, 21, 23, or 25.

83. A method according to claim 81, wherein the protein has an amino acid motif selected from the group consisting of the motif of SEQ ID. No. 69, the motif of SEQ ID. No. 70, the motif of SEQ ID. No. 71, the motif of SEQ ID. No. 72, the motif of SEQ ID. No. 73, the motif of SEQ ID. No. 74, the motif of SEQ ID. No. 75, and combinations thereof.

84. A method according to claim 81, wherein the protein has an amino acid motif selected from the group consisting of the motif of SEQ ID. No. 76, the motif of SEQ ID. No. 77, the motif of SEQ ID. No. 78, the motif of SEQ ID. No. 79, the motif of SEQ ID. No. 80, the motif of SEQ ID. No. 81, the motif of SEQ ID. No. 82, the motif of SEQ ID. No. 83, the motif of SEQ ID. No. 84, and combinations thereof.

85. A method according to claim 81, wherein the protein has an amino acid sequence of SEQ ID. No. 85.

86. A method according to claim 81, wherein the protein has an amino acid sequence of SEQ ID. No. 86.

87. A method according to claim 81, wherein a transgenic plant is provided.

88. A method according to claim 81, wherein a transgenic plant seed is provided and said method further comprises:
planting the plant seeds under conditions effective for plants to grow from the planted plant seeds.

89. A method according to claim 81, wherein the plant is selected from the group consisting of alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.

90. A method according to claim 81, wherein the plant is selected from the group consisting of Arabidopsis thaliana, Saintpaulia, petunia, pelargonium, poinsettia, chrysanthemum, carnation, and zinnia.

91. A method according to claim 81, wherein the hypersensitive response elicitor treatment is for imparting disease resistance.

92. A method according to claim 81, wherein the hypersensitive response elicitor treatment is for enhancing plant growth.

93. A method according to claim 81, wherein the hypersensitive response elicitor treatment is for controlling insects.

94. A method according to claim 81, wherein the hypersensitive response elicitor treatment is for imparting stress tolerance.

95. A method according to claim 81, wherein the transgenic plant or plant seed is further transformed with a second nucleic acid encoding a hypersensitive response elicitor, wherein expression of the second nucleic acid effects the hypersensitive response elicitor treatment.

96. A method according to claim 81, wherein the hypersensitive response elicitor treatment comprises:
applying a hypersensitive response elicitor to the plant or plant seed.

97. A method according to claim 96, wherein the hypersensitive response elicitor is applied in isolated form.

98. A method of imparting disease resistance, enhancing growth, controlling insects, and/or imparting stress resistance to plants comprising:
providing a transgenic plant or transgenic plant seed transformed with a DNA construct effective to silence expression of a nucleic acid molecule according to claim 16.

99. A method according to claim 98, wherein the protein is from a monocot.

100. A method according to claim 99, wherein the protein is from rice, barley, wheat, or maize.

101. A method according to claim 98, wherein the protein is from a dicot.

102. A method according to claim 101, wherein the protein is from Arabidopsis thaliana, cotton, soybean, tomato, potato, grapefruit, apple, tobacco, or grape.

103. A method according to claim 98, wherein either: (1) the protein has an amino acid sequence of SEQ. ID. Nos. 1, 6, 8, 12, 18, 26, 28, 30, 32, or 34; (2) the nucleic acid hybridizes to a nucleotide sequence of SEQ. ID. Nos. 2, 7, 9, 13, 19, 27, 29, 31, 33, 35, or 39 under stringent conditions of hybridization in buffer comprising 20% formamide in 0.9M saline/0.09M SSC at a temperature of 42° C.; (3) the nucleic acid comprises a nucleotide sequence of SEQ. ID. Nos. 2, 7, 9, 13, 19, 27, 29, 31, 33, or 35; (4) the nucleic acid hybridizes to a nucleotide sequence of SEQ. ID. No. 3 under stringent conditions of hybridization in buffer comprising 20% formamide in 0.9M saline/0.09M SSC at a temperature of 42° C.; (5) the nucleic acid comprises a nucleotide sequence of SEQ. ID. No. 3; (6) the protein has an amino acid sequence of SEQ. ID. Nos. 4, 10, 14, 16, 20, 22, or 24; (7) the nucleic acid hybridizes to the nucleotide sequence of SEQ. ID. Nos. 5, 11, 15, 17, 21, 23, or 25 under stringent conditions of hybridization buffer comprising 20% formamide in 0.9 M saline/0.09M SSC at a temperature of 42° C.; (8) the nucleic acid comprises a nucleotide sequence of SEQ. ID. Nos. 5, 11, 15, 17, 21, 23, or 25; (9) the protein has an amino acid motif selected from the group consisting of the motif of SEQ ID. No. 69, the motif of SEQ ID. No. 70, the motif of SEQ ID. No. 71, the motif of SEQ ID. No. 72, the motif of SEQ ID. No. 73, the motif of SEQ ID. No. 74, the motif of SEQ ID. No. 75, and combinations thereof; (10) the protein has an amino acid motif selected from the group consisting of the motif of SEQ ID. No. 76, the motif of SEQ ID. No. 77, the motif of SEQ ID. No. 78, the motif of SEQ ID. No. 79, the motif of SEQ ID. No. 80, the motif of SEQ ID. No. 81, the motif of SEQ ID. No. 82, the motif of SEQ ID. No. 83, the motif of SEQ ID. No. 84, and combinations thereof; (11) the protein has an amino acid sequence of SEQ ID. No. 85; or (12) the protein has an amino acid sequence of SEQ ID. No. 86.

104. A method according to claim 98, wherein a transgenic plant is provided.

105. A method according to claim 98, wherein a transgenic plant seed is provided and said method further comprises:
planting the plant seeds under conditions effective for plants to grow from the planted plant seeds.

106. A method according to claim 98, wherein the plant is selected from the group consisting of alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.

107. A method according to claim 98, wherein the plant is selected from the group consisting of Arabidopsis thaliana, Saintpaulia, petunia, pelargonium, poinsettia, chrysanthemum, carnation, and zinnia.

108. A method according to claim 98, wherein the transgenic plant or plant seed is further transformed with a second nucleic acid encoding a hypersensitive response elicitor, wherein expression of the second nucleic acid effects a hypersensitive response elicitor treatment.

109. A method according to claim 98 further comprising:
applying a hypersensitive response elicitor to the plant or plant seed.

110. A method according to claim 98, wherein the hypersensitive response elicitor is applied in isolated form.

111. A method according to claim 98, wherein disease resistance is imparted to plants.

112. A method according to claim 98, wherein enhanced growth is imparted to plants.

113. A method according to claim 98, wherein insect control is imparted to plants.

114. A method according to claim 98, wherein stress resistance is imparted to plants.

115. A method according to claim 98, wherein the DNA construct is an antisense nucleic acid molecule to a nucleic acid molecule encoding a receptor in plants for plant pathogen hypersensitive response elicitors.

116. A method according to claim 98, wherein the DNA construct is transcribable to a first nucleic acid encoding a receptor in plants for plant pathogen hypersensitive response elicitors coupled to a second nucleic acid encoding the inverted complement of the first nucleic acid.

117. A method of imparting disease resistance, enhancing growth, controlling insects, and/or imparting stress resistance to plants comprising:
providing a transgenic plant or transgenic plant seed transformed with the nucleic acid molecule according to claim 16.

118. A method according to claim 117, wherein either: (1) the protein has an amino acid sequence of SEQ. ID. Nos. 1, 6, 8, 12, 18, 26, 28, 30, 32, or 34; (2) the nucleic acid hybridizes to a nucleotide sequence of SEQ. ID. Nos. 2, 7, 9, 13, 19, 27, 29, 31, 33, 35, or 39 under stringent conditions of hybridization in buffer comprising 20% formamide in 0.9M saline/0.09M SSC at a temperature of 42° C.; (3) the nucleic acid comprises a nucleotide sequence of SEQ. ID. Nos. 2, 7, 9, 13, 19, 27, 29, 31, 33, or 35; (4) the nucleic acid hybridizes to a nucleotide sequence of SEQ. ID. No. 3 under stringent conditions of hybridization in buffer comprising 20% formamide in 0.9M saline/0.09M SSC at a temperature of 42° C.; (5) the nucleic acid comprises a nucleotide sequence of SEQ. ID. No. 3; (6) the protein has an amino acid sequence of SEQ. ID. Nos. 4, 10, 14, 16, 20, 22, or 24; (7) the nucleic acid hybridizes to the nucleotide sequence of SEQ. ID. Nos. 5, 11, 15, 17, 21, 23, or 25 under stringent conditions of hybridization in buffer comprising 20% formamide in 0.9 M saline/0.09M SSC at a temperature of 42° C.; (8) the nucleic acid comprises a nucleotide sequence of SEQ. ID. Nos. 5, 11, 15, 17, 21, 23, or 25; (9) the protein has an amino acid motif selected from the group consisting of the motif of SEQ ID. No. 69, the motif of SEQ ID. No. 70, the motif of SEQ ID. No. 71, the motif of SEQ ID. No. 72, the motif of SEQ ID. No. 73, the motif of SEQ ID. No. 74, the motif of SEQ ID. No. 75, and combinations thereof; (10) the protein has an amino acid motif selected from the group consisting of the motif of SEQ ID. No. 76, the motif of SEQ ID. No. 77, the motif of SEQ ID. No. 78, the motif of SEQ ID. No. 79, the motif of SEQ ID. No. 80, the motif of SEQ ID. No. 81, the motif of SEQ ID. No. 82, the motif of SEQ ID. No. 83, the motif of SEQ ID. No. 84, and combinations thereof; (11) the protein has an amino acid sequence of SEQ ID. No. 85; or (12) the protein has an amino acid sequence of SEQ ID. No. 86.

119. A method according to claim 117, wherein a transgenic plant is provided.

120. A method according to claim 117, wherein a transgenic plant seed is provided and said method further comprises:
planting the plant seeds under conditions effective for plants to grow from the planted plant seeds.

121. A method according to claim 117, wherein the plant is selected from the group consisting of alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, radish, spinach, onion, garlic, eggplant, pepper, celery, carrot, squash, pumpkin, zucchini, cucumber, apple, pear, melon, citrus, strawberry, grape,,raspberry, pineapple, soybean, tobacco, tomato, sorghum, and sugarcane.

122. A method according to claim 117, wherein the plant is selected from the group consisting of Arabidopsis thaliana, Saintpaulia, petunia, pelargonium, poinsettia, chrysanthemum, carnation, and zinnia.

123. A method according to claim 117, wherein disease resistance is imparted.

124. A method according to claim 117, wherein plant growth is enhanced.

125. A method according to claim 117, wherein insects are controlled.

126. A method according to claim 117, wherein stress tolerance is imparted.

127. A method according to claim 117, wherein the protein is from a monocot.

128. A method according to claim 127, wherein the protein is from rice, barley, wheat, or maize.

129. A method according to claim 117, wherein the protein is from a dicot.

130. A method according to claim 129, wherein the protein is from Arabidopsis thaliana, cotton, soybean, tomato, potato, grapefruit, apple, tobacco, or grape.


SEQUENCE LISTING (86 sequences)

SEQ ID NO: 1 - protein, 284 aa, Arabidopsis thaliana
MATSSTFSSLLPSPPALLSDHRSPPPSIRYSFSPLTTPKSSRLGFTVPEKRNLAANSSLVEVSIGGESDPPPSSSGSGGDDKQIALLKLKLLSVVSGLNRGLVASVDDLERAEVAAKELETAGGPVDLTDDLDKLQGKWRLLYSSAFSSRSLGGSRPGLPTGRLIPVTLGQVFQRIDVFSKDFDNIAEVELGAPWPFPPLEATATLAHKFELLGTCKIKITFEKTTVKTSGNLSQIPPFDIPRLPDSFRPSSNPGTGDFEVTYVDDTMRITRGDRGELRVFVIA

SEQ ID NO: 2 - DNA, 1000 bp, Arabidopsis thaliana
tttttccttctcaacaatggcgacttcttctactttctcgtcactactaccttcaccaccagctcttctttccgaccaccgttctcctccaccatccatcagatactccttttctcccttaactactccaaaatcgtctcgtttgggtttcactgtaccggagaagagaaacctcgctgctaattcgtctctcgttgaagtatccattggcggagaaagtgacccaccaccatcatcatctggatcaggaggagacgacaagcaaattgcattactcaaactcaaattacttagtgtagtttcgggattaaacagaggacttgtggcgagtgttgatgatttagaaagagctgaagtggctgctaaagaacttgaaactgctgggggaccggttgatttaaccgatgatcttgataagcttcaagggaaatggaggctgttgtatagtagtgcgttctcttctcggtctttaggtggtagccgtcctggtctacctactggacgtttgatccctgttactcttggccaggtgtttcaacggattgatgtgtttagcaaagattttgataacatagcagaggtggaattaggagccccttggccatttccgccattagaagccactgcgacattggcacacaagtttgaactcttaggcacttgcaagatcaagataacatttgagaaaacaactgtgaagacatcgggaaacttgtcgcagattcctccgtttgatatcccgaggcttcccgacagtttcagaccatcgtcaaaccctggaactggggatttcgaagttacctatgttgatgataccatgcgcataactcgcggggacagaggtgaacttagggtattcgtcattgcttaattctcaaagctttgacatgtaaagataaataaatactttctgcttgatgcagtctcatgagttttgtacaaatcatgtgaacatataaatgcgctttataagtaaatgagtgtcttgttcaatgaatca

SEQ ID NO: 3 - DNA, 4260 bp, Arabidopsis thaliana
aattagaaaaattaacaaccaacatctagttagaatatttaatttgcaccaatgtcttcgagtatagtgaaaaaaatagaagatcgaatatcgaatagtacgtatagaatcatctagatccattcgaactaacgtctacttttcttttccagcattaacatgtagcttgtcattagcatttacatgttgcaaataacacaaattgggaaattgaaagactaaaaaaccttgtacagcagatggtttaacacgtggattcatggacacaaacagaaaacggcagaactaagcacaaaaacgtcaactaagcatatcaaagcttttaatgcaagcctaatataaacacaagtggttatccataatctgttcttaatctcttgcagtagttatcttttcattattcgcaattcgcaattctatattcttatatttcaacttgttcttcttccaaattgtaattatatctacatcgtcttagcttgaccattatagctccagtaccaagttctcttcttaactttaatatcagctactattctcatactgtaaatatcttttgttcaccaaacatatatttcgaaccaaactgctaaaagcttatcataaattgcagttctagccacacaattttgcagttccaaccattaaatgccacaaaatttggacgatttcttaagacaagaagaacatagcaaccaaaccttattgattaaatatgaaatgtctccataaaactgggagatttccccaaataaagagaacacggcaaatgttcacgtaatctccaagatgaatgtttaattttttctttcagaaaaaaacaaaaaaacttaactcaatatagacaactagaatggataccaactaagcaaaagaaattcaaaagacaaatatatattggatatgaagttacattattttcaaactttatatactactaaaagcctaaaaatttgttctaaaatgatatccaaataaatggaaggcatgaatgtcatatgactaaaagagaaaaacacacctgtatataagtattggatcatgctgcctccgagtgacaaaacatacgatgtgggtctttattgggccatacttaaatggaaaaaggagaaaaaaaattgggcaatgtctatggtcgaaatttatatgttttacatcaataaaatcaatatttaattttatatatgtgggtcttaatctagtattatctacatagattaaaatcaaagtactgcatatggtccataataatacaaccaaagcaaattaaaattttgtggcacaaaacgacatcattttactcagaaagtaatatgcaatttcgtttacgcacacacgtatacgcgctaataacccgtggtgcttctcaaatcacataataattaaagtcttcttcttcttcttcttctctacaaattatctcactctcttcgtttttttttccttctcaacaatggcgacttcttctactttctcgtcactactaccttcaccaccagctcttctttccgaccaccgttctcctccaccatccatcagatactccttttctcccttaactactccaaaatcgtctcgtttgggtttcactgtaccggagaagagaaacctcgctgctaattcgtctctcgttgaagtatccattggcggagaaagtgacccaccaccatcatcatctggatcaggaggagacgacaagcaaattgcattactcaaactcaaattacttgtgagtctgattcaaaccaatcggtgaaattataagaaattggtttcgtttctttggaattagggtttatattactgttaagattcgattatagagtgaattttgggaagatttttcagatttgatttgtgatgtgttgtgttgtgagaaattgcagagtgtagtttcgggattaaacagaggacttgtggcgagtgttgatgatttagaaagagctgaagtggctgctaaagaacttgaaactgctgggggaccggttgatttaaccgatgatcttgataagcttcaagggaaatggaggctgttgtatagtagtgcgttctcttctcggtctttaggtggtagccgtcctggtctacctactggacgtttgatccctgttactcttggccaggtaattcttgaatcattgctctgtttttacccgtcaagattcggtttttcgggtaagttgttgaggagtttatgtgcatggtctagtctatcatcaatagtcttgcttgagtttgaatggggctgagctaagaatctagctttctgaggttacaatttggtaatgtcatcttatactcgaaagcaaacttttttctatattgtcaagtttatgtgtacggtctggtctatcattggtagtctttgttgagcttgaatggtgaggagcttagaatctagcaatgtcatctactccttaatcatttttttctatattgccaagtttatgtgtacggtcttagtcaatcatctttattcttggttgagtttgaatggtgatgagcttagaatctagctttctttggtttaaatttggcaaagaaccatacctgaatcggtagaaagcaaacttctttaatattatctcttgtttctgaatcattaaaacaggtgtttcaacggattgatgtgtttagcaaagattttgataacatagcagaggtggaattaggagccccttggccatttccgccattagaagccactgcgacattggcacacaagtttgaactcttaggtttgcatttccctttctctcattaagtttatcgaattgtgtaagagcaaaataacttatctgtatctttgacatttatggggaaaacaggcacttgcaagatcaagataacatttgagaaaacaactgtgaagacatcgggaaacttgtcgcagattcctccgtttgatatcccgaggcttcccgacagtttcagaccatcgtcaaaccctggaactggggatttcgaagttacctatgttgatgataccatgcgcataactcgcggggacagaggtgaacttagggtattcgtcattgcttaattctcaaagctttgacatgtaaagataaataaatactttctgcttgatgcagtctcatgagttttgtacaaatcatgtgaacatataaatgcgctttataagtaaatgagtgtcttgttcaatgaatcatatgaaagaatttgtatgactcagaaaattggacaatgatatagaccttccaaattttgcaccctctaatgtgagatattagtgattttttcttaggttggtagagagaacggattggcaaaaaaatatcgaaggtcaatgattaacagcaaaaccatatcttgatgattcaaaatatagagttaacaagcaaagatgagacaatcttatacgagagagctaaaacaaatggattccaaatccagcaagtacaaaaatcgcagaaaataagatgaaaccaacttaaaacagagatgttccctttcccttcttgtcaccaccgatctcgaaatgcttgcacctctgaaataaacaacaaaccaacacaatgtaagcaaattaccaagttacaaatccggtataatgaactgatctatgttctatgcaccttgataggacgctgcgaaaagtgcttgcagctttgacactgaagcctcaaaacaatcttcttcgtggtcttagcctgttaacaagattcacaagatgtatctcagtccaaaactgagactattggaatgtctgtttcctcacagctcacttccaaaattctactataaatggttccttaaaactacctcatttcaactaactagacctaattcaaactgaaaaaacaatcaatgcatgataatcaatgttacctttttgtggaagacaggcttagtctgaccaccataaccagattgtttacggtcataacgacgctttccttgagcagcaagactgtctttacccttcttgtattgggtaaccttgtgcaaagtatgctttttgcattccttgttcttacagtaagtgttctttgtctttggaatgttcaccttcaaaattcataaaatcaaaaatgaatcactcacacacatacaaaatcaagagacttttaaggttaatcaaaatacaaacatcatttagattgaaaacttttatgatagatctgaaaaacaatacaataaatcaatcaaccatgtattgttgttcttcaaagtcaacgaactttacaaattccaaaatcacatcgaaagagaagaaacaatttaccattttcgcgt

SEQ ID NO: 4 - protein, 203 aa, Oryza sativa
VAALKVKLLSAVSGLNRGLAGSQEDLDRADAAARELEAAAGGGPVDLERDVDKLQGRWRLVYSSAFSSRTLGGSRPGPPTGRLLPITLGQVFQRIDVVSKDFDNIVDVELGAPWPLPPVELTATLAHKFEIIGTSSIKITFDKTTVKTKGNLSQLPPLEVPRIPDNLRPPSNTGSGEFEVTYLDGDTRITRGDRGELRVFVIS

SEQ ID NO: 5 - DNA, 613 bp, Oryza sativa
cgtggctgcgctcaaagtcaagcttctgagcgcggtgtccgggctgaaccgcggcctcgcggggagccaggaggatcttgaccgcgccgacgcggcggcgcgggagctcgaggcggcggcgggtggcggccccgtcgacctggagagggacgtggacaagctgcaggggcggtggaggctggtgtacagcagcgcgttctcgtcgcggacgctcggcggcagccgccccggcccgcccaccggccgcctcctccccatcaccctcgggcaggtgtttcagaggatcgatgttgtcagcaaggacttcgacaacatcgtcgatgtcgagctcggcgcgccatggccgctgccgccggtggagctgacggcgaccctggctcacaagtttgagatcatcggcacctcgagcataaagatcacattcgacaagacgacggtgaagacgaaggggaacctgtcccagctgccgccgctggaggtccctcgcatcccggacaacctccggccgccgtccaacaccggcagcggcgagttcgaggtgacctacctcgacggcgacacccgcatcacccgcggggacagaggggagctcagggtgttcgtcatctcgtga

SEQ ID NO: 6 - protein, 277 aa, Gossypium
MASSSFLLESPASIFSSSSIKAHLYLPKPYPFIVSVKRRRSERKRNPVLKSAVGDVSVVDTPPPPPPPPQDAKSELISSLKLKLLGIVSGLNRGLAANQDDLGKADDAAKELETVAGPVDLLTDLDKLQGRWKLIYSSAFSSRTLGGSRPGLPTGRLLPVTLGQVFQRIDVISKDFDNIAEIELGAPWPLPPLEVTATLAHKFEIIGSSKIKITFEKTSVKTRGTFSQLPSLDVPRIPDALRPPSNPGSGDFDVTFIDADTRITRGDRGELRVFVIS

SEQ ID NO: 7 - DNA, 1064 bp, Gossypium
aaagctttcttgcaaaaagctccgaaaaagggccagcaaaagccacttgagagccaatggcttcttcaagttttcttctagaatctccggcgtctatcttctcttcttcctccattaaagctcatctctatctcccgaaaccctacccttttattgttagcgtgaaacggcgccgttcggaaaggaagcgaaaccctgttttaaaatcggctgttggagatgtctccgtcgttgacaccccaccgccgccgccgcctccacctcaagatgctaaatctgaactcatttcttctttgaagcttaaattactgggtattgtttctgggctgaatagaggtcttgctgcgaaccaagatgatctcggaaaagcagatgatgccgccaaggaactcgaaacggttgctggacctgtggacttattgaccgatcttgataagctgcaagggagatggaaactgatatacagcagtgcattctcgtctcgtacactcggcgggagccgtcctggacttcccactggaaggttgctccctgtaactctcggccaggtttttcagagaattgatgtcataagcaaagattttgataatatagcagaaattgaattgggagctccatggccattacctccacttgaagttactgctaccttagctcacaaatttgaaatcataggatcttcaaagatcaaaataacattcgagaaaacgagtgtgaaaactagagggaccttttctcagcttccgtcattggatgtacctcggattcccgacgctttgaggcctccatctaatccagggagcggcgactttgatgttaccttcattgatgccgatacccgaatcaccagaggagatagaggtgagcttagggtttttgtcatctcataaattagtaagcacatctaatatcaaagctcgtatgcactctcattacttcatatattgtctgtatgtgtatatatcattgggggtgatccgtaactttttgtagaattaatattttaatgtaattacgaatattatgtatgtaaattttcgaatcaatttaatagtttaatcgtg

SEQ ID NO: 8 - protein, 265 aa, Glycine max
MASLNLLPHPPLFSSFLHRPHCNTHLLLTPKPSQRRPSLVVKSTVGVADPSPSSSSYAGDTSDSISSLKLNLLSAVSGLNRGLAASEDDLRKADDAAKELEAAGGLVDLSLGLDNLQGRWKLIYSSAFSSRTLGGSRPGPPIGRLLPITLGQVFQRIDILSKDFDNIVELQLGAPWPLPPLEATATLAHKFELIGSSKIKIVFEKTTVKTAGNLSQLPPLEVPRIPDALRPPSNTGSGEFEVTYLDSDTRITRGDRGELRVFVIA

SEQ ID NO: 9 - DNA, 1075 bp, Glycine max
ggcacgaggctccaatccatggcttccctgaacctccttccccaccctccacttttctcttctttccttcacagaccacactgcaacacccatcttcttctcacaccaaaaccttctcaacgaaggccttctcttgtggtcaaatctactgtgggtgtggctgacccttctccatcttcttcttcctacgctggggatacctctgattccatctcttctttgaagctcaatctgctgagtgctgtttctgggctaaatagaggccttgctgcaagcgaagacgatcttcgaaaggcagatgatgctgctaaggaacttgaagctgctggaggacttgtggatctctcgcttggtcttgacaatttgcaaggaagatggaaactcatttatagcagcgcattttcgtctcgaacccttggtggaagccgtcctggtcctcccataggaagactccttcctattactcttggacaggtttttcaacgaattgacatcttgagcaaagattttgataacatagtggagcttcaactaggtgctccatggcccctaccaccccttgaagcgactgccacattagctcacaaatttgaactcataggatcttcaaagataaagatagtatttgagaaaaccactgtgaagacagctgggaatttgtcacagttgccaccattggaggtgcctcggattcccgatgcattgaggcctccatctaatacgggaagcggtgaatttgaagttacatatcttgactcggatactcgcatcacaagaggagacagaggcgagctaagggtctttgtgattgcttgagttcctggtgaatgcaactatgcactatgcattttctctgttggacttaaaaaaaaaaggtttcaacaccttgtgccatcattttgtttagttttttcctcctgatggtatttgttctaagttcttcaatattgtaaacatgatggaattaaactctactatatagttccaaggaagcagggtactttttgtttaagtgtaacatatttcttttttaaggaataattgcttacagatcattagatatggatacttgaat

SEQ ID NO: 10 - protein, 276 aa, Hordeum vulgare
MAMASPSWSSCCTSTSTHSLPGPPASSKGRNPWRASSGRRSASGGKRQQKLSIRAVAAPSAAVDYSDTGAGAGDIPSKIKLLSAVAGLNRGLAASQEDLDRADAAARQLEAAAPAPVDLAKDLDKLQGRWRLVYSSAFSSRTLGGSRPGPPTGRLLPITLGQVFQRIDVVSQDFDNIVELELGAPWPLPPVEATATLAHKFEITGIASIKINFDKTTVKTNGNLSQLPLLEVPRIPDSLRPPTSNTGSGEFNVTYLDDDTRITRGDRGELRVFVVT

SEQ ID NO: 11 - DNA, 1129 bp, Hordeum vulgare
gccggtcggcacccaactggaggttcagtttcctcgttgctctcctccattgattgaccgcctccttccctgaggcgcacggtacacggacggcacccatggccatggcatcgccgtcgtggtcatcctgctgcacctcaacctccacccattctctgcccggtcctcccgcgagcagccagggcaggaacccgtggcgggcaagcagcggcaggaggagcgccagcggagggaagaggcagcagaagctgtccatccgcgcggtggccgcaccgtcggccgcggtggactactcggacaccggcgccggcgccggcgacatcccctcgctgaaaatcaagctgccgagcgccgtcgccgggctgaaccggggcctcgctgcgagccaggaggacctggaccgggcggacgcggcggcgcggcagctcgaggcggcggcgccggcccccgtggacctcgccaaggatctcgacaagctgcaggggcggtggaggctggtctacagcagcgccttctcgtcgcggacgctcggcggcagccgccccggcccgcccaccggtcgcctcctccccatcaccctcggccaggtgttccagaggatcgacgtggtgagccaggacttcgacaacatcgtggagctcgagctcggcgccccgtggccgctgccgccggtggaggccacggccacgctggcacacaagtttgagatcaccggaatcgcgagtatcaagatcaatttcgacaagacgacggtgaagacgaacgggaacctgtcccagctgccgctgctggaggtgccccgcatcccggatagcctcaggccgccgacttccaacaccgggagcggcgagttcaacgtgacctatctcgacgacgacacccgcatcacccgaggggacaggggggagctcagggtgttcgtcgtcacatgagcttttttttgctgcgatctctctctttgtagtgctccaactttttttggcccgtaaaacaagagtcttgtactagttctatatatgccttttgttttggggttcacccgtccatccgcgggaaacatctatcgtgacgactgttcgatgtataagcggagtcgtccgatttacgcggttccgtcgtcttttcgaac

SEQ ID NO: 12 - protein, 276 aa, Lycopersicon esculentum
MASLLHSRLPLSHNHSLSNSCQSFPCHLPGRSKRSTQRLLEERSYDSKRSLVCQSGIDEVTFIEPPGSKEAEAELIGSLKLKLLSAVSGLNRGLAASEDDLKKADEAAKELESCAGAVDLAADLDKLQGRWKLIYSSAFSSRTLGGSRPGPPTGRLLPITLGQVFQRIDVLSKDFDNIVELELGAPWPFPPVEATATLAHKFELIGSSTIKIIFEKTTVKTTGNLSQLPPLEVPRIPDQFRPPSNTGSGEFEVTYIDSDTRVTRGDRGELRVFVIS

SEQ ID NO: 13 - DNA, 1026 bp, Lycopersicon esculentum
tcgatcctttttctgaaattcaagctcaaccatggcttctctacttcattcgagacttcccctttctcacaatcattctttatcaaattcttgccaatctttcccatgtcatctcccaggaagaagcaagagaagtactcaaagattattagaggaaaggagctatgacagcaagagaagtttagtttgccagtcgggtattgatgaagtcacttttattgagccacctggtagtaaagaagctgaagcggagcttattgggtctctcaaactcaagttattgagtgctgtttctgggctaaacagaggtcttgctgcaagtgaagatgatctaaagaaggcggatgaggctgccaaggagctagaatcttgtgcaggagctgtagatctcgcagctgatcttgataaacttcaagggaggtggaaattgatatacagcagtgcattctcatctcgtactcttggtggaagtcgtcctggaccccccactggaagacttcttcccatcactcttggtcaggtatttcaaagaatcgatgtactgagcaaagattttgacaacatagtggagcttgaattaggtgctccgtggcctttcccgcctgttgaagcaactgccactttagcccacaaatttgaacttataggatcatctacgattaagattatattcgagaaaactacagtgaagacaactggaaatttatcacagctcccaccattagaagtgcctcgcataccagatcagttcaggccaccatcaaatacaggaagtggtgagtttgaagttacctacatcgattctgatacacgagtaacaaggggagacagaggagagcttagagttttcgttatctcataagttaagctgcaatgaatatagtcttcctacaatgttttgttgctacaatttcatgtaacaacatatcaaatgtgtagatatgctcaacattattctgctggtcacagctatcaaatctgtaatgctactgcaaattcaaatctgtatacagtaaatttgacatc

SEQ ID NO: 14 - protein, 270 aa, Oryza sativa
MAAAVASSCCASTSARPLVRRAGSRNGKLWWAGGVRKARLLSISATAAAPSGVDYAAGTGAAADDDAVAALKVKLLSAVSGLNRGLAGSQEDLDRADAAARELEAAAGGGPVDLERDVDKLQGRWRLVYSSAFSSRTLGGSRPGPPTGRLLPITLGQVFQRIDVVSKDFDNIVDVELGAPWPLPPVELTATLAHKFEIIGTSSIKITFDKTTVKTKGNLSQLPPLEVPRIPDNLRPPSNTGSGEFEVTYLDGDTRITRGDRGELRVFVIS

SEQ ID NO: 15 - DNA, 1123 bp, Oryza sativa
tcgccattgattttctctgtctgctctgctgctcgcttgcttgcgctgtccggtttagctctgtctagctaggtagactgcggccatggcggcggcggtggcgtcgtcttgctgcgcctcgaccagcgctcgcccactggttcgccgcgccgggagcaggaacgggaagctgtggtgggcgggtggtgtcaggaaggcgcggctgctgtccatctccgccacggccgcggcgccgtcgggcgtggactacgcggcgggcaccggcgccgccgccgacgacgacgccgtggctgcgctcaaagtcaagcttctgagcgcggtgtccgggctgaaccgcggcctcgcggggagccaggaggatcttgaccgcgccgacgcggcggcgcgggagctcgaggcggcggcgggtggcggccccgtcgacctggagagggacgtggacaagctgcaggggcggtggaggctggtgtacagcagcgcgttctcgtcgcggacgctcggcggcagccgccccggcccgcccaccggccgcctcctccccatcaccctcgggcaggtgtttcagaggatcgatgttgtcagcaaggacttcgacaacatcgtcgatgtcgagctcggcgcgccatggccgctgccgccggtggagctgacggcgaccctggctcacaagtttgagatcatcggcacctcgagcataaagatcacattcgacaagacgacggtgaagacgaaggggaacctgtcccagctgccgccgctggaggtccctcgcatcccggacaacctccggccgccgtccaacaccggcagcggcgagttcgaggtgacctacctcgacggcgacacccgcatcacccgcggggacagaggggagctcagggtgttcgtcatctcgtgatcggacggacgcgttcgcgacataggtatgcggcttgcgattctgaaactgaaactgaagcgcacacacggttttgtgttctttctctgctactagtagatcctcactctcttgatctgaccatctttgtactatacttcagtattgttcgtgcgttctgtattgttatagattttgcagatattcaacaagtagagggaaatatgtcaaaatgagaaatcgagg

SEQ ID NO: 16 - protein, 269 aa, Oryza sativa
MAAAVASSCCASTSARPLVRRAGSRSGKLWWAGGGRKARLLSISATAAAPSGVDYAAGTGAADDDAVAALKVKLLSAVSGLNRGLAASQEDLDRADAAARELEAAAGGGPVDLEGDMDKLQGRWRLVYSSAFSSRTLGGSRPGPPTGRLLPITLGQVFQRIDVVSKDFDNIVDVELGAPWPLPPVELTATLAHKFEIIGTSSIKITFDKTTVKTKGNLSQLPPLEVPRIPDNLRPPSNTGSGEFEVTYLDGDTRITRGDRGELRVFVIS

SEQ ID NO: 17 - DNA, 1112 bp, Oryza sativa
tcgccattgattttctctgtctgctctgctgctcgcttgcttgcgctgtccggtttagctctgtctagctaggtagactggcggccatggcggcggcggtggcgtcgtcttgctgcgcctcgaccagcgctcgcccactggttcgccgcgccgggagcaggagcgggaagctgtggtgggcgggtggtgggaggaaggcgcggctgctgtccatctccgccacggccgcggcgccgtcgggcgtggactacgcggcgggcaccggcgccgccgacgacgacgccgtggctgcgctcaaagtcaagcttctgagcgcggtgtccgggctgaaccgcggcctcgcggcgagccaggaggatcttgaccgggccgacgcggcggcgcgggagctcgaggcggcggcgggcggcgggcccgtcgacctggagggggacatggacaagctgcaggggcggtggaggctggtgtacagcagcgcgttctcgtcgcggacgctcggcggcagccgccccggcccgcccaccggccgcctcctccccatcaccctcggccaggtgtttcagaggatcgatgttgtcagcaaggacttcgacaacatcgtcgatgtcgagctcggcgcgccatggccgctgccgccggtggagctgacggcgacgctggctcacaagtttgagatcatcggcacctcgagcataaagatcacattcgacaagacgacggtgaagacgaaggggaacctgtcccagctgccgccgctggaggtccctcgcatcccggacaacctccggccgccgtccaacaccggcagcggcgagttcgaggtgacctacctcgacggcgacacccgcatcacccgcggggacagaggggagctcagggtgttcgtcatctcgtgatcggacggacgcgttcgcgacataggtatgcggcttgcgattctgaaactgaaactgaagcgcacacacggttttgtgttctttctctgctactagtagatcctcactctcttgatctgaccatctttgtactatacttcagtattgttcgtgcgttctgtattgttatagattttgcagatattcaacaagtagagggaaatatgccaaaatgag

SEQ ID NO: 18 - protein, 275 aa, Solanum tuberosum
MASLLHSRLPLSHNHSLSNSCQSFPCHLPGRSKRSTQRFFEERSYDSKRALICQSGIDEVTFRLPGSKEAKAELIGSLKLKLLSAVSGLNRGLAASEDDLKKADEAAKELESCAGAVDLAADLDKLQGRWKLIYSSAFSSRTLGGSRPGPPTGRLLPITLGQVFQRIDVLSKDFDNIVELELGAPWPFPPVEATATLAHKFELIGSSTIKIVFEKTTVKTTGNLSQLPPIEVPRIPDQFRPPSNTGNGEFEVTYIDSDTRVTRGDRGELRVFVIS

SEQ ID NO: 19 - DNA, 1078 bp, Solanum tuberosum
  unsure (951): N at position 951 is A, C, T, or G
ctaccaccaatcaaactccacaaaagatcgatcctttttctgaaattcaagctcaaccatggcttctctacttcattctagacttcccctttctcacaatcattctttatcaaattcttgccaatctttcccctgtcatctcccaggaagaagcaagagaagtactcaaagattctttgaggaaaggagctatgatagcaagagagccttaatttgtcagtcgggtattgatgaagtcacttttaggctacctggtagtaaagaagctaaagctgagcttattgggtctctcaaactcaagttattgagtgctgtttctgggctaaacagaggtcttgctgcaagtgaagatgatctaaagaaggcggatgaggctgccaaggagctggaatcttgtgcaggagctgtagatctcgcagctgatcttgataagcttcaagggaggtggaaattgatatacagcagtgcattctcatctcgtactcttggtggaagtcgtcctggcccccccactggaagacttcttcccatcactcttggtcaggtatttcaaagaattgatgtactaagcaaggattttgacaacatagtggagcttgaattaggtgctccgtggcctttcccacctgttgaagcaactgccactttagcccacaaatttgaacttataggatcatctacaattaagattgtattcgaaaaactcagtgaagacaactggaaatttatcacagttgccaccaatagaagtgcctccataccagatcagttcaggccaccatcaaatacaggaaatggtgagtttgaagttacctatatcgattctgatacacgtgtaacaaggggagacagaggagagcttagagttttcgttatctcataagttaagctgcaataaatatagttttcctacaatattttgttgctacaatttcatgtaacaacatatcnaatgtatagatatgctcaacattattctgctggtcaaagctagcaaatttgtaatgctactgcaaattcaaatctgtatacagtaaatttgacatgtgatggagttatgcagtgagatttcnanaat

SEQ ID NO: 20 - protein, 277 aa, Triticum
MAMASPSWSSCCASTSTRPLPSPPASSKSRNPWRASSGRRSASGGKRRQQLSIRAVAAPSSAVDYSDTAAGAGDVPSLKIKLLSAVAGLNRGLAASQEDLDRADAAARQLEAAAPAPVDLAKDLDKLQGRWRLVYSSAFSSRTLGGSRPGPPTGRLLPITLGQVFQRIDVVSQDFDNIVELELGAPWPLPPVEATATLAHKFEITGIASIKINFDKTTVKTKGNLSQLPLLEVPRIPDSLRPTTSNTGSGEFDVTYLDDGTRITRGDRGELRVFVVS

SEQ ID NO: 21 - DNA, 1056 bp, Triticum
gaattcggcacgagctgacctcttgccggtcggcgcccaattgaaaatttcttttctttttgctctcctgatcgattgactgcctcacggacggtgcccatggccatggcatcgccgtcgtggtcatcttgctgcgcctccacctccacccgtcctctgcctagcccccccgcgagcagcaagagcaggaacccatggcgggcaagcagcggcaggaggagcgccagcggagggaagagacgacagcagctgtccatccgcgcggtggccgcaccgtcgtcggcggtggactactcggacaccgccgccggcgccggcgacgtcccctcgctgaaaatcaagctgctgagcgcggtcgccgggctgaaccggggcctcgcggcgagccaggaggacctggaccgggcggacgcggcggcgaggcagctcgaggcggcggcaccggcccccgtggacctcgccaaggacctcgacaagctgcaggggcggtggaggctggtctacagcagcgccttctcgtcgcggacgctcggcggcagccgccccggcccgcccaccggccgcctcctccccatcaccctcggccaggtgttccagaggatcgacgtggtcagccaggacttcgacaacatcgtggagctcgagctcggcgcgccgtggccgctgccgccggtcgaggccacggccacgctggcgcacaagtttgagatcaccggaatcgcgagtatcaagatcaatttcgacaagacgacggtgaagaccaaagggaacctgtcccagctgcctctgctggaggtgccccgcatcccggatagcctccggcctacgacgtccaacaccgggagcggcgagttcgacgtgacctacctcgacgacggcacccgcatcacccgaggggacaggggggagctcagggtgttcgtcgtctcatgagctgatattttttttgttgatgttgctgctgctttctctctccgtgtactgcttcaacctttttgcccctaaacagaagtcttgaactagttctatgtctatttttgccggagtagtatcgtg

SEQ ID NO: 22 - protein, 275 aa, Triticum
MAAPSWSSCCASTSTRPLPSPPASSKGGNPWRASSGRRSASGGKRQQQLSIRAVAAPSSAVDYSDTGAGAADVPSLKIKLLSAVAGLNRGLAASQEDLDRADAAARQLEAAAPAPVDLAKDLDKLQGRWRLVYSSAFSSRTLGGSRPGPPTGRLLPITLGQVFQRIDVVSQDFDNIVELELGAPWPLPPVEATATLAHKFEITGIASIKINFDETTVKTNGNLSQLPLLEVPRIPDSLRPPASNTGSGEFDVTYLDDDTRITRGDRGELRVFVIA

SEQ ID NO: 23 - DNA, 1205 bp, Triticum
actagtgattcgcggatccatatgctgcgtttgctggctttgatgaaactcgtgctcgtctctgacctctggccggtcggcacccaactgaaaatatcttttctcgttgctctcctcgatcgattgactgcttcaccggacggtgcccgtggccatggcagcgccgtcgtggtcatcttgctgcgcctccacctccacccgtcctctgcctagccctcccgcgagcagcaagggcgggaacccatggcgggcaagcagcggcaggaggagcgccagcggagggaagaggcagcagcagctgtccatccgcgcggtggccgcgccgtcgtcggcggtggactactcggacaccggcgccggcgccggcgacgtcccctcgctgaaaatcaagctgctgagcgcggtggccgggctgaaccggggcctcgcggcgagccaggaggacctggaccgggcggacgcggcggcgaggcagctcgaggcggcggcgccggcccccgtggacctcgccaaggacctcgacaagctgcaggggcggtggaggctggtctacagcagcgccttctcgtcgcggacgctcggcggtagccgccccggcccgcccaccggccgcctgctccccatcaccctcggccaggtgttccagaggatcgacgtggtgagccaggacttcgacaacatcgtggagctcgagctcggcgcgccgtggccgctgccgccggtggaggccacggccacgctggcacacaagtttgagatcaccgggatcgcgagtatcaagatcaatttcgacgagacgacggtgaagacgaatgggaacctgtcccagctgcctctgctggaggtgccccgcatcccggatagcctccggccgccggcgtccaacaccgggagcggcgagttcgacgtgacctacctcgacgacgacacccgcatcacccgaggggacaggggggagctcagggtgttcgtcatcgcatgagcttgatctttgcttgagatctctgtctctgtactgcttcactttttttgccccgaaacagaagtctttgtctagttctatgtcttcttttgccggcgtagtattgtgatataggctaacgtgcgttcttcacctatgggattaactttttctctctagcagattattacgtccggttatttcgttttggttttattatgttggcttaagttttaattatgtg

SEQ ID NO: 24 - protein, 272 aa, Zea mays
MAATWSSSCCAATASSSALLRHARVKSAPWVAGASRSSYRQRRRRRELSIRATAAAPPPPVVYADAGADNVASLKIKLLSAVSGLNRGLAASQEDLDRADAAARELEAAAGCPVDLSRDLDKLQGRWRLLYSSAFSSRTLGGSRPGPPTGRLLPITLGQVFQRIDVVSRDFDNIVELELGAPWPLPPLEATATLAHKFEIIGTSGIKITFEKTTVKTKGNLSQLPPLEVPRIPDNLRPPSNTGSGEFEVTYLDDDTRVTRGDRGELRVFVIA

SEQ ID NO: 25 - DNA, 1218 bp, Zea mays
ccaccacaaatattcttcccgccacgatccctctcatccggaagaaaggggaaaaaaactcgcctttttctctctgctggttcaagaacgccatggaagatctcgagcgctcgctgtgattcctgcgagtacccaagcccaaccaagccctggcccggcagccattctcttcgcgccacatcgcacgacctccccgaagcagacgtgcccgctgcccgtccgtcccccgtggccatggccgcgacgtggtcttcgtcttgctgcgccgcgaccgcgtcgagcagcgctctgcttcgtcatgcccgcgtcaagagcgcgccttgggtagccggtgccagccggagtagctacaggcagcggcggcggcggcgggagctgtccatccgcgccacggccgcggcgccgccgccgcccgtggtctacgcggacgccggcgccgacaacgtggcctcgctgaagatcaagctcctgagcgcggtgtccgggctgaaccgtggcctggcagcgagccaggaggacctggaccgcgcggacgcggcggcgcgggagctggaggcggcggcggggtgccccgtcgacctcagcagggacctcgataagctgcagggccggtggcggctgctgtacagcagcgcgttctcttcgcggacgctcggcggcagccgccttggcccgcccaccggccgcctcctccccatcacgctcggccaggtgttccagcggatcgacgtggtgagccgcgacttcgacaacatcgtggagctggagctcggcgcgccgtggcctctgccgccgctcgaggccacggcgacgctggcgcacaagttcgagatcatcgggacctcgggcatcaagatcacgttcgagaagacgacggtgaagaccaagggcaacctgtcgcagcttcctccgctggaggtgccccgcatcccggacaacctccgccccccgtccaacaccgggagcggcgagttcgaggtgacctacctcgacgacgacacgcgcgtcacccgcggggacaggggggagctcagggtgtttgtcatcgcgtgacctgatcgcgcttcggcgccgttctgctggtccgtgagattgccatccttcttcctccctgttgctccagtagatttgttggtttcttcgtctgaccaatgtataccgttctgttcttccgtgaactgaatctgcgattaacttagtaactatcttgtgtggttt

SEQ ID NO: 26 - protein, 285 aa, Citrus paradisi
MASLTLTPLFHSPTFLSSNTNTHTVTKKLSFPSPTRRRLLVNGKEYRSRRRSLVLRRSAVDDVPVLDPPPPPPPDSSESDKTELIASLKLKLLSAVSGLNRGLAANTDDLQKADAAAKELEAVGGPVDLSVGLDRLQGKWRLLYSSAFSSRTLGGNRPGPPTGRLLPITLGQVFQRIDILSKDFDNIAELELGVPWPLPPVEVTATLAHKFELIGSSNIKIIFEKTTVKTTGNLSQLPPLELPRFPDALRRPSDTRSGEFEVTYLDNDTRITRGDRGELRVFVIT

SEQ ID NO: 27 - DNA, 1101 bp, Citrus paradisi
ttcgattgccagacgctgcgtttgctggctttgatgaaacctctttcattccctgctggccacaaacacacgccgacattgaaactccccccacccacatcatggcttctctgactctaacccctctttttcattcaccaacatttctttccagcaatactaacacacacacagtcacaaaaaaactgtcttttccatctccaacgcgacgtcgtctgcttgttaatggtaagagtatcgaagtagaagaagaagccttgttttgaggaggtcagccgttgatgacgttcctgttcttgacccaccactcctcctcctcccgattcttcagaaagcgacaaaactgagctcattgcttctttgaagctcaagttgcttagtgctgtttctgggctgaacagaggtcttgctgcaaacacagatgatctgcagaaggcagacgctgctgcaaaagagcttgaggctgttggaggaccagtagacctctcggttggtctcgatagactacaagggaaatggagactactgtacagcagtgcattctcatctcgcactctaggtggaaatcggcctggacctcccactggaaggctactccccataactcttggccaggtctttcaacggattgacatcttaagcaaagattttgataacatagcagaacttgaattgggtgttccatggcccctgccaccagttgaagtgactgccacattagcccataaatttgaactcataggatcatcaaatattaaaataatatttgagaagacaactgtaaagacaacagggaacttatcacagcttccaccccttgagttacctcgttttccagatgcattaaggcgtccatctgacacaagaagtggtgaatttgaggtgacatacctcgataatgatacccgcattaccagaggagacagaggcgagctaagagttttcgtgatcacttaggttccttacatccgtacagtttccagcttgtatctacattattttctgatgattatatacacaaagtggtaaaaagaagccccgtgaaaagcagttcttcctggatcaagtgaatcattgcacaattatatatttttcatgcgc

SEQ ID NO: 28 - protein, 282 aa, Pyrus malus
MAMASLSSLPHSLHSSPSTSSANYVIPSKPPCPKRLRFGSSNRRHTKSFAPRAAVDEVSVLEPPPPQPPSSGSKTTPNPELVASLKLNLLSAVSGLNRGLAASGEDLQKAEAAAKEIEAAGGPVDLSTDLDKLQGRWKLIYSSAFSSRTLGGSRPGPPTGRLLPITLGQVFQRIDIFSKDFDNIVELELGAPWPLPPVEATATLAHKFELIGSSRVKIIFEKTTVKTTGNLSQLPPLELPKLPEGLRPPSNPGSGEFDVTYLDADIRITRGDRDELRVFVVS

SEQ ID NO: 29 - DNA, 1138 bp, Pyrus malus
ggctttgatgaaatttcctttctactttctagccatggccatggcttctttgagctctctccctcactctctacattcctcgccttctacttcttctgcaaactatgttattccaagcaaaccaccctgcccaaaacgcctccgttttggttcgtcaaatcgccgtcacaccaaaagctttgctccgagagcagctgtggacgaggtttctgttctcgaaccgccgccaccacagccgccgtcttccggaagcaaaaccacgcccaaccctgaacttgtagcgtctttaaagctcaacctattgagtgctgtttctgggctaaatagaggtcttgcagcatcgggagaggatctacaaaaggcagaagctgctgccaaggagattgaagctgctggaggtccagtggatctctcaactgatcttgataaactgcaagggagatggaaattgatatatagcagtgcattttcttctcgtactctaggtgggagccgtcctggacctcccaccggaaggctactcccaattaccttaggccaggtatttcaacggattgacatcttcagcaaagactttgataacatagtggagcttgaactaggtgctccatggcccctgccacccgttgaagcaactgccactttggcccacaaatttgaactcataggatcttccagggttaagatcatttttgagaaaactactgtgaagactactggaaacttatcgcagcttcctccattagagttacctaagttaccggaaggactacgacctccgtctaacccaggaagtggtgaatttgacgttacctaccttgatgctgatatccgcatcacaagaggagatagagacgagctaagggtttttgttgtttcatagtttcttgttagtttcttttcctacttccaatgtatctccatctgttttgccttgcgtcttcttggtgtcgtttgatcatatgttgttacttccaattgttgtatgcatgaaccggtggatggaagttccaggaaatgttcaacgaggaacaacactgtatacatgtaaattttgtaatcgataaagtgaatcgtctttgtcacttggattgtatctgcattgccttttcaagtgatatctatatgagttttaggc

SEQ ID NO: 30 - protein, 276 aa, Nicotiana tabacum
MASLLQYSTLPLSNNHCSSSLPSLTCHLSKRSNRNTQKLLEKKKYHIKKSLICQSGIDELAFIELPGTKEAKAELIGSLKLKLLSAVSGLNRGLAASEEDLKKADAAAKELESCAGAVDLSADLDKLQGRWKLIYSSAFSGRTLGGSRPGPPTGRLLPITLGQVFQRIDVLSKDFDNIVELELGAPWPLPPAELTATLAHKFELIGSSTIKITFEKTTVKTTGILSQLPPFEVPRIPDQLRPPSNTGSGEFEVTYIDSDTRVTRGDRGELRVFVIS

SEQ ID NO: 31 - DNA, 1044 bp, Nicotiana tabacum
attcacaaacctttccaaatattgagctgaaattaaagctcaacaatggcttctctacttcagtactctacacttcctctttctaataatcattgttcatcttcgttaccatctttaacttgtcatctctcaaaaagaagcaatagaaatactcaaaaattattagagaaaaagaagtatcatatcaagaaaagcttaatttgccagtcgggtattgatgaactcgcattcattgagttacctggtactaaagaagctaaagctgaacttattgggtctctcaaactcaagttattgagtgctgtttctgggctaaacagaggtcttgctgcgagcgaagaagacctaaagaaggcggatgctgctgccaaggagctagaatcctgtgcaggagctgtagatctctcagctgatctcgataaacttcaagggaggtggaaattgatatacagcagtgcattctcaggtcgcactcttggaggaagtcgtcctggaccccccaccggaagacttcttcccattactcttggtcaggtatttcaaagaattgatgtgctaagcaaggattttgacaacatagtggagcttgaattaggtgctccttggcctttaccacctgctgagttgactgccactttagcccacaaatttgaactgataggatcatccacgattaagattacattcgagaaaactactgtgaagacaaccggaatcttatcacagctcccaccatttgaggtgcctcggataccagatcaactcaggccaccatctaatacaggaagtggtgagtttgaagttacctatattgattctgatacacgcgtaacaaggggagacagaggagagcttagagttttcgttatctcataagatggaatgcaatagatatagttttcctacaatattttgttgctacaatttcatgtacaatatatcaaatgtatagatatgctcaacattattctgctggtccatatctagcaaagttgtaatgttactgcaaatttgaatctgtatacagtaaactcgattttgcga

SEQ ID NO: 32 - protein, 291 aa, Vitis
MTSLLHPLTSFSLSPSPPPPLSSSSSSTITITCALPSNLRSSDRRRLRTTSKPYTWTSGLPKRSFVLRSTLDEVSVLDPPPPPEDSTADLLSSLKLKLLSAVSGLNRGLAAIEDDLQKADAAAKELEAAGGTVDLSIDLDKLQGRWKLIYSSAFSSRTLGGSRPGPPTGRLLPITLGQVFQRIDIVSKDFDNIVDLQIGVPWPLPPIELTATLAHKFELIGTSSIKITFEKTTVKTTGNLSQLPPLEVPRIPDALRPPSNTGSGEFEVTYLDADTRITRGDRGELRVFVIA

SEQ ID NO: 33 - DNA, 1038 bp, Vitis
accgccagccaactatgacttctctcctccatcctctcacctctttctccctttctccatcaccaccaccgcccctttcttcttcttcttcttctactattactatcacgtgtgctcttcccagtaacctacgttcttcagaccgacgtcgtcttagaacaacatcaaaaccttatacgtggacatcgggcctgcccaagagaagctttgtcctgaggtcaacccttgatgaggtctctgttcttgacccccctcctccccctgaagactccacggccgatcttctttcgtctctcaagctgaaactactgagtgctgtgtctggtctaaatagaggacttgctgcaatcgaggatgatcttcagaaggcagatgctgctgccaaagagcttgaagctgctggaggaactgttgacctctcaattgatcttgataaacttcagggaagatggaaattgatatatagcagtgcgttctcatcccgtactctaggtgggagccgtcctggacctcccactggaaggctactccctataactctgggccaggtatttcaaaggattgacattgtaagcaaagattttgacaatatagtagatctccagataggtgtcccatggccccttccgccaattgaactcactgccacattagcccacaagtttgaactcataggaacttccagcattaaaataacattcgagaaaacaactgtgaagacaacaggaaacctgtcgcagctgccaccattggaggtacctcggatcccagatgcattgaggccaccatctaatacaggaagtggcgaatttgaggttacataccttgatgctgatacccgcatcaccagaggagacaggggtgagcttagagtttttgtcattgcataaactctaagcactcgtcaccatgactcacaattgaagaaaataccatatccaatccccttttcttcttgtcattttgtaaacagtcccctgtttcttactgtttgtagggaacatgtcttgttacatataactgtaaattcattttttt

SEQ ID NO: 34 - protein, 292 aa, Vitis
MTSLLHPLTSFSLSPSPPPPLSFSSSSSTITITCALPSNLRSSDRRRLRTTSKPYTWTSGLPKRSFVLRSTLDEVSVLDPPPPPEDSTADLLSSLKLKLLSTVSGLNRGLAAIEDDLQKADAAAKELEAAGGTVDLSIDLDKLQGRWKLIYSSAFSSRTLGGSRPGPPTGRLLPITLGQVFQRIDIVSKDFDNIVDLQIGAPWPLPPIELTATLAHKFELIGTSSIKITFEKTTVKTTGNLSQLPPLEVPRIPDALRPPSNTGSGEFEVTYLDADTRITRGDRGELRVFVIA

SEQ ID NO: 35 - DNA, 1055 bp, Vitis
accgccagccaactatgacttctctcctccatcctctcacctctttctccctttctccatcaccaccaccgcccctttctttttcttcttcttcttctactattactatcacgtgtgctcttcccagtaacctacgttcttcagaccgacgtcgtcttagaacaacatcaaaaccttatacgtggacatcgggcctgcccaagagaagctttgtcctgaggtcaacccttgatgaggtctctgttcttgacccccctcctccccctgaagactccacggccgatcttctttcgtctctcaaactgaaactactgagtactgtgtctggtctaaatagaggacttgctgcaatcgaggatgatcttcagaaggcagatgctgctgccaaagagcttgaagctgctggaggaactgttgacctctcaattgatcttgataaacttcagggaagatggaaattgatatatagcagtgcgttctcatcccgtactctaggtgggagccgtcctggacctcccactggaaggctactccctataactctggggcaggtatttcaaaggattgacattgtaagcaaagattttgacaatatagtagatctccagataggtgccccatggccccttccgccaattgaactcactgccacattagcccacaagtttgaactcataggaacttccagcattaaaataacattcgagaaaacaactgtgaagacaacaggaaacctgtcgcagcttccaccattggaggtacctcggatcccagatgcattgaggccaccatctaatacaggaagtggcgaatttgaggttacataccttgatgctgatacccgcatcaccagaggagacaggggtgagcttagagtttttgtcattgcataaactctacactcgtcaccatgactcacaattgaagaaaatacaatatccaatccccttttcttcttgtcattttgtaaactgtcccctgtttcttactgtttgtagggaacatgtcttgttacataactgtaaattcattttttctacatttgatctttacag

SEQ ID NO: 36 - protein, 26 aa, Xanthomonas campestris pv. glycines
TLIELMIVVAIIAILAAIALPAYQDY

SEQ ID NO: 37 - protein, 114 aa, Xanthomonas campestris pv. pelargonii
MDSIGNNFSNIGNLQTMGIGPQQHEDSSQQSPSAGSEQQLDQLLAMFIMMMLQQSQGSDANQECGNEQPQNGQQEGLSPLTQMLMQIVMQLMQNQGGAGMGGGGSVNSSLGGNA

SEQ ID NO: 38 - DNA, 342 bp, Xanthomonas campestris pv. pelargonii
atggactctatcggaaacaacttttcgaatatcggcaacctgcagacgatgggcatcgggcctcagcaacacgaggactccagccagcagtcgccttcggctggctccgagcagcagctggatcagttgctcgccatgttcatcatgatgatgctgcaacagagccagggcagcgatgcaaatcaggagtgtggcaacgaacaaccgcagaacggtcaacaggaaggcctgagtccgttgacgcagatgctgatgcagatcgtgatgcagctgatgcagaaccagggcggcgccggcatgggcggtggcggttcggtcaacagcagcctgggcggcaacgcc

SEQ ID NO: 39 - DNA, 205 bp, Arabidopsis thaliana
gatcaagataacatttgagaaaacaactgtgaagacatcgggaaacttgtcgcagattcctccgtttgatatcccgaggcttcccgacagtttcagaccatcgtcaaaccctggaactggggatttcgaagttacctatgttgatgataccatgcgcataactcgcggggacagaggtgaacttagggtattcgtcattgcttaa

SEQ ID NO: 40 - DNA, 24 bp, Artificial Sequence
  Description of Artificial Sequence  primer
acgagaaggcgttgctgtagacca

SEQ ID NO: 41 - DNA, 24 bp, Artificial Sequence
  Description of Artificial Sequence  primer
agcttgattttcagcgaggggatg

SEQ ID NO: 42 - DNA, 22 bp, Artificial Sequence
  Description of Artificial Sequence  primer
acctcaacctccacccattctc

SEQ ID NO: 43 - DNA, 23 bp, Artificial Sequence
  Description of Artificial Sequence  primer
cttctcgaacgtgatcttgatgc

SEQ ID NO: 44 - DNA, 25 bp, Artificial Sequence
  Description of Artificial Sequence  primer
atgttgtcgaagtcgcggctcacca

SEQ ID NO: 45 - DNA, 20 bp, Artificial Sequence
  Description of Artificial Sequence  primer
tagctccttggcagcctcat

SEQ ID NO: 46 - DNA, 23 bp, Artificial Sequence
  Description of Artificial Sequence  primer
gtgacttcatcaatacccgactg

SEQ ID NO: 47 - DNA, 24 bp, Artificial Sequence
  Description of Artificial Sequence  primer
gctcaaccatggcttctctacttc

SEQ ID NO: 48 - DNA, 24 bp, Artificial Sequence
  Description of Artificial Sequence  primer
cacttttattgagccacctggtag

SEQ ID NO: 49 - DNA, 24 bp, Artificial Sequence
  Description of Artificial Sequence  primer
ctgaccaagagtgatgggaagaag

SEQ ID NO: 50 - DNA, 24 bp, Artificial Sequence
  Description of Artificial Sequence  primer
caagagtacgagatgagaatgcac

SEQ ID NO: 51 - DNA, 22 bp, Artificial Sequence
  Description of Artificial Sequence  primer
acgagaaggcgctgctgtagac

SEQ ID NO: 52 - DNA, 21 bp, Artificial Sequence
  Description of Artificial Sequence  primer
gcgctcagcagcttgattttc

SEQ ID NO: 53 - DNA, 22 bp, Artificial Sequence
  Description of Artificial Sequence  primer
ttgctctcctcgatcgattgac

SEQ ID NO: 54 - DNA, 22 bp, Artificial Sequence
  Description of Artificial Sequence  primer
atcgccgtcgtggtcatcttgc

SEQ ID NO: 55 - DNA, 21 bp, Artificial Sequence
  Description of Artificial Sequence  primer
ccgatgatctcaaacttgtga

SEQ ID NO: 56 - DNA, 27 bp, Artificial Sequence
  Description of Artificial Sequence  primer
gtccttgctgacaacatcgatcctctg

SEQ ID NO: 57 - DNA, 26 bp, Artificial Sequence
  Description of Artificial Sequence  primer
tcgccattgattttctctgtctgctc

SEQ ID NO: 58 - DNA, 25 bp, Artificial Sequence
  Description of Artificial Sequence  primer
gaagcttgactttgagcgcagccac

SEQ ID NO: 59 - DNA, 27 bp, Artificial Sequence
  Description of Artificial Sequence  primer
gacgccgtggctgcgctcaaagtcaag

SEQ ID NO: 60 - DNA, 25 bp, Artificial Sequence
  Description of Artificial Sequence  primer
gtggactacgcggcgggcaccggcg

SEQ ID NO: 61 - DNA, 23 bp, Artificial Sequence
  Description of Artificial Sequence  primer
tcraayttrtgngcnarngtngc

SEQ ID NO: 62 - protein, 8 aa, Artificial Sequence
  Description of Artificial Sequence  peptide
ATLAHKFE

SEQ ID NO: 63 - DNA, 17 bp, Artificial Sequence
  Description of Artificial Sequence  primer
atnckytgraanacytg

SEQ ID NO: 64 - protein, 6 aa, Artificial Sequence
  Description of Artificial Sequence  peptide
QVFQRI

SEQ ID NO: 65 - DNA, 23 bp, Artificial Sequence
  Description of Artificial Sequence  primer
gtnwsnggnytnaaymgnggnyt

SEQ ID NO: 66 - protein, 8 aa, Artificial Sequence
  Description of Artificial Sequence  peptide
VSGLNRGL

SEQ ID NO: 67 - DNA, 23 bp, Artificial Sequence
  Description of Artificial Sequence  primer
ggncargtnttycarmgnathga

SEQ ID NO: 68 - protein, 8 aa, Artificial Sequence
  Description of Artificial Sequence  peptide
GQVFQRID

SEQ ID NO: 69 - protein, 6 aa, Artificial Sequence
  Description of Artificial Sequence  peptide
GLNRGL

SEQ ID NO: 70 - protein, 6 aa, Artificial Sequence
  Description of Artificial Sequence  peptide
YSSAFS

SEQ ID NO: 71 - protein, 10 aa, Artificial Sequence
  Description of Artificial Sequence  peptide
TLGQVFQRID

SEQ ID NO: 72 - protein, 5 aa, Artificial Sequence
  Description of Artificial Sequence  peptide
DFDNI

SEQ ID NO: 73 - protein, 9 aa, Artificial Sequence
  Description of Artificial Sequence  peptide
TATLAHKFE

SEQ ID NO: 74 - protein, 5 aa, Artificial Sequence
  Description of Artificial Sequence  peptide
TRGDR

SEQ ID NO: 75 - protein, 6 aa, Artificial Sequence
  Description of Artificial Sequence  peptide
ELRVFV

SEQ ID NO: 76 - protein, 6 aa, Artificial Sequence
  Description of Artificial Sequence  peptide
AAXXLE

SEQ ID NO: 77 - protein, 14 aa, Artificial Sequence
  Description of Artificial Sequence  peptide
LQGXWXLXYSSAFS

SEQ ID NO: 78 - protein, 5 aa, Artificial Sequence
  Description of Artificial Sequence  peptide
RXLGG

SEQ ID NO: 79 - protein, 17 aa, Artificial Sequence
  Description of Artificial Sequence  peptide
GRLXPXTLGQVFQRIDX

SEQ ID NO: 80 - protein, 10 aa, Artificial Sequence
  Description of Artificial Sequence  peptide
TATLAHKFEX

SEQ ID NO: 81 - protein, 5 aa, Artificial Sequence
  Description of Artificial Sequence  peptide
TXVKT

SEQ ID NO: 82 - protein, 5 aa, Artificial Sequence
  Description of Artificial Sequence  peptide
VTXXD

SEQ ID NO: 83 - protein, 7 aa, Artificial Sequence
  Description of Artificial Sequence  peptide
RXTRGDR

SEQ ID NO: 84 - protein, 7 aa, Artificial Sequence
  Description of Artificial Sequence  peptide
ELRVFVX

SEQ ID NO: 85 - protein, 92 aa, Artificial Sequence
  Description of Artificial Sequence  consensus
XGLNRGLXYSSAFSXXXXXXXXXXXXXXXXXXXTLGQVFQRIDXXXXDFDNIXXXXXXXXXXXXXXXXTATLAHKFEXTRGDRXELRVFVXX

SEQ ID NO: 86 - protein, 146 aa, Artificial Sequence
  Description of Artificial Sequence  consensus
XGLNRGLXXXXXXXXXXXXAAXXLEXLQGXWXLXYSSAFSXRXLGGXXXXXXXGRLXPXTLGQVFQRIDXXXXDFDNIXXXXXXXXXXXXXXXXTATLAHKFEXXXXXXXXXXXXXTXVKTXVTXXDXXXRXTRGDRXELRVFVXX